(19) United States
(12) Patent Application Publication
(10) Pub. No.: US 2025/0026807 A1
(43) Pub. Date: Jan. 23, 2025

(54) Immune Cell Binding Polypeptides

(71) Applicant: Cue Biopharma, Inc., Boston, MA (US)

(72) Inventor: Anish Suri, Boston, MA (US)

(21) Appl. No.: 18/905,993

(22) Filed: Oct. 3, 2024

Related U.S. Application Data
(63) Continuation of application No. PCT/US2023/065427, filed on Apr. 6, 2023.
(60) Provisional application No. 63/328,563, filed on Apr. 7, 2022.

(51) Int. Cl. C07K 14/74; A61K 38/00; C07K 14/705

(52) U.S. Cl. CPC C07K 14/70539; C07K 14/70532; A61K 38/00; C07K 2319/30

(57) ABSTRACT

The present disclosure provides immune cell binding polypeptides (ICBPs) that include a binding moiety that can bind to ILT2 and/or ILT4 polypeptides, at least one immunoinhibitory polypeptide, and a scaffold polypeptide. The ICBPs are useful for inducing immunological tolerance. The present disclosure provides methods of inducing immunological tolerance.

### CROSS-REFERENCE

This application claims the benefit of U.S. Provisional Patent Application No. 63/328,563, filed Apr. 7, 2022, which application is incorporated herein by reference in its entirety.

### INCORPORATION-BY-REFERENCE OF MATERIAL ELECTRONICALLY SUBMITTED

A Sequence Listing is provided herewith as a Sequence Listing XML, “CUEB-147WO_SEQ_LIST” created on Apr. 5, 2023 and having a size of 188,080 bytes. The contents of the Sequence Listing XML are incorporated by reference herein in their entirety.

### INTRODUCTION

Inflammatory diseases and disorders are conditions in which an abnormal or otherwise de-regulated inflammatory response contributes to the etiology or severity of disease. Examples of such diseases include autoimmune diseases. Inflammatory diseases and disorders may be characterized by infiltration of mononuclear cells at a site of inflammation. Examples of mononuclear cells include lymphocytes (e.g., T lymphocytes), and cells of the mononuclear phagocyte system including monocytes, macrophages, dendritic cells, and microglial cells. Pharmaceutical compositions that can induce immunological tolerance thus may be useful for the treatment of autoimmune diseases and disorders or other conditions that involve an adverse immune response such as metabolic disorders.

### SUMMARY

The present disclosure provides immune cell binding polypeptides (ICBPs) that include a binding moiety that can bind to ILT2 and/or ILT4 receptor polypeptides on cells, at least one immunoinhibitory polypeptide, and a scaffold polypeptide. The ICBPs are useful for inducing immunological tolerance. The present disclosure provides methods of inducing immunological tolerance.

### BRIEF DESCRIPTION OF THE DRAWINGS

FIG. 1A-1B provide an amino acid sequence of a wild-type human β2M polypeptide (FIG. 1A; SEQ ID NO:1) and an amino acid sequence of a β2M polypeptide with an R12C substitution (FIG. 1B; SEQ ID NO:2).

FIG. 2A-2M provide amino acid sequences of immunoglobulin Fe polypeptides (SEQ ID NOs:3-15, respectively).

FIG. 3 provides an HLA-G consensus sequence (SEQ ID NO:16).

FIG. 4A-4F provide amino acid sequences of HLA-G heavy chains (SEQ ID NOs:17-22, respectively).

FIG. 5A-5B provide amino acid sequences of single-chain ICBP polypeptides (SEQ ID NOs:23 and 29, respectively).

FIG. 6A-6C—provide amino acid sequences of polypeptides of (i) construct 4601+4602 (FIG. 6A) (SEQ ID NO:30), (ii) construct 4607+4602 (FIG. 6B) (SEQ ID NO:33), and iii) construct 4607-2+4602 (FIG. 6C) (SEQ ID NO:35).

FIG. 7 depicts binding of construct 4601+4602 and construct 4607+4602 to ILT2, ILT4, and PD1.

FIG. 8A-8F provide amino acid sequences of ILT2 polypeptides (SEQ ID NOs:36-41, respectively).

FIG. 9A-9E provide amino acid sequences of ILT4 polypeptides (SEQ ID NOs:42-46, respectively).

FIG. 10A-10F provide amino acid sequences of CD155 isoforms (FIG. 10A and FIG. 10B; SEQ ID NOs:47 and 48, respectively), amino acid sequences of CD112 isoforms (FIG. 10C and FIG. 10D; SEQ ID NOs:49 and 50, respectively), an amino acid sequence of FGL1 (FIG. 10E; SEQ ID NO:51), and an amino acid sequence of the extracellular portion of LSECtin (FIG. 10F; SEQ ID NO:52).

FIG. 11A-11D provide amino acid sequences of wild-type and variant PD-L1 polypeptides (SEQ ID NOs:34 and 53-55, respectively).

FIG. 12A-12G provide amino acid sequences of TGF-β polypeptides (SEQ ID NOs:56-62, respectively).

FIG. 13A and FIG. 13B provide amino acid sequences of IL-12α and IL-27β polypeptides (SEQ ID NOs:63 and 64, respectively).

FIG. 14A-14C provide amino acid sequences of KIR2DL4 (SEQ ID NOs:65-67, respectively).

### DEFINITIONS

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. Furthermore, as used herein, a “polypeptide” refers to a protein that includes modifications, such as deletions, additions, and substitutions (generally conservative in nature as would be known to a person in the art) to the native sequence, as long as the protein maintains the desired activity. These modifications can be deliberate, as through site-directed mutagenesis, or can be accidental, such as through mutations of hosts that produce the proteins, or errors due to polymerase chain reaction (PCR) amplification or other recombinant DNA methods. References herein to a specific residue or residue number in a known polypeptide are understood to refer to the amino acid at that position in the wild-type polypeptide. To the extent that the sequence of the wild-type polypeptide is altered, either by addition or deletion of one or more amino acids, one of ordinary skill will understand that a reference to the specific residue or residue number will be correspondingly altered so as to refer to the same specific amino acid in the altered polypeptide, which would be understood to reside at an altered position number. For example, if an HLA-G polypeptide is altered by the addition of one amino acid at the N-terminus, then a reference to position 84 or a specific residue at position 84, will be understood to indicate the amino acids that are at position 85 on the altered polypeptide. Likewise, a reference herein to substitution of a specific amino acid at a specific position, e.g., Y84, is understood to refer to a substitution of an amino acid for the amino acid at position 84 in the wild-type polypeptide. A Y84C substitution is thus understood to be a substitution of Cys residue for the Tyr residue that is present in the wild-type sequence. If, e.g., the wild-type polypeptide is altered to change the amino acid at position 84 from its wild-type amino acid to an alternate amino acid, then the substitution for the amino acid at position 84 will be understood to refer to the substitution for the alternate amino acid. If in such case the polypeptide is also altered by the addition or deletion of one or more amino acids, then the reference to the substitution will be understood to refer to the substitution for the alternate amino acid at the altered position number. A reference to a “non-naturally occurring Cys residue” in a polypeptide, e.g., an HLA-G polypeptide, means that the polypeptide comprises a Cys residue in a location where there is no Cys in the corresponding wild-type polypeptide. This can be accomplished through routine protein engineering in which a cysteine is substituted for the amino acid that occurs in the wild-type sequence.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined BLAST, which is available over the world wide web at sites including ncbi.nlm.nili.gov/BLAST, ebi.ac.uk/Tools/msa/tcoffee/, ebi.ac.uk/Tools/msa/muscle/, mafft.cbrc.jp/alignment/software/. Unless otherwise stated, “sequence identity” as referred to herein is determined by BLAST (Basic Local Alignment Search Tool) with the default settings selected.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine.

“T cell” includes all types of immune cells expressing CD3, including T-helper cells (CD4⁺ cells), cytotoxic T-cells (CD8⁺ cells), T-regulatory cells (Treg), and natural killer (NK)-T cells.

The term “immunoinhibitory polypeptide”, as used herein, means a polypeptide that specifically binds a cognate polypeptide (e.g., a receptor or a co-receptor) on an immune cell (e.g., a T cell, a dendritic cell, etc.), thereby providing a signal that inhibits an immune cell response, including, but not limited to, proliferation, activation, secretion of inflammatory cytokines, and the like. As discussed herein, an immunoinhibitory polypeptide can include, but is not limited to, wild-type or variants of wild-type polypeptides such as a cytokine (e.g., IL-10, IL-35, IL-2), PD-L1, Fas-L, a LAG-3 ligand, a TIGIT ligand (e.g., CD155), and TGF-β.

As used herein the term “in vivo” refers to any process or procedure occurring inside of the body.

As used herein, “in vitro” refers to any process or procedure occurring outside of the body.

“Heterologous,” as used herein, means a nucleotide or polypeptide that is not found in the native nucleic acid or protein, respectively.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system.

The terms “recombinant expression vector,” or “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

As used herein, the term “affinity” refers to the equilibrium constant for the reversible binding of two agents (e.g., an antibody and an antigen) and is expressed as a dissociation constant (K_{D}). As used herein, the term “avidity” refers to the resistance of a complex of two or more agents to dissociation after dilution.

The term “binding,” as used herein (e.g., with reference to binding of a first polypeptide to a second polypeptide (e.g., where the first polypeptide is an ICBP of the present disclosure, and where the second polypeptide is an ILT2 polypeptide)), refers to a non-covalent interaction between two molecules. Non-covalent binding refers to a direct association between two molecules, due to, for example, electrostatic, hydrophobic, ionic, and/or hydrogen-bond interactions, including interactions such as salt bridges and water bridges. “Affinity” refers to the strength of non-covalent binding, increased binding affinity being correlated with a lower K_{D}. “Specific binding” generally refers to binding of a ligand to a moiety that is than its designated binding site or receptor. “Non-specific binding” generally refers to binding of a ligand to a moiety other than its designated binding site or receptor. “Covalent binding” or “covalent bond,” as used herein, refers to the formation of one or more covalent chemical binds between two different molecules.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may or may not be predisposed to acquiring the disease or symptom but has not yet been diagnosed as having it; (b) inhibiting the disease or one or more symptoms associated with the disease, e.g., arresting its development; and/or (c) relieving the disease, i.e., causing regression of the disease. The therapeutic agent may be administered before, during and/or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient,” are used interchangeably herein and refer to any mammalian subject for whom diagnosis, treatment, or therapy is desired. Mammals include, e.g., humans, non-human primates, rodents (e.g., rats; mice), lagomorphs (e.g., rabbits), ungulates (e.g., cows, sheep, pigs, horses, goats, and the like), etc. Unless otherwise indicated, the terms “individual,” “subject,” “host,” and “patient,” refer to a human.

Unless indicated otherwise, the term “substantially” is intended to encompass both “wholly” and “largely but not wholly”. For example, an Ig Fc that “substantially does not induce ADCC” means an Ig Fc that induces no ADCC at all or that largely does not induce ADCC.

As used herein, the term “about” used in connection with an amount indicates that the amount can vary by 10% of the stated amount. For example, “about 100” means an amount of from 90-110. Where about is used in the context of a range, the “about” used in reference to the lower amount of the range means that the lower amount includes an amount that is 10% lower than the lower amount of the range, and “about” used in reference to the higher amount of the range means that the higher amount includes an amount 10% higher than the higher amount of the range. For example, from about 100 to about 1000 means that the range extends from 90 to 1100.

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

As used herein and in the appended claims, the singular forms “a,” “an,” and “the” include plural referents unless the context clearly dictates otherwise. Thus, for example, reference to an “ICBP” includes a plurality of such ICBPs and reference to “the immunoinhibitory polypeptide” includes reference to one or more immunoinhibitory polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

The publications discussed herein are provided solely for their disclosure. Nothing herein is to be construed as an admission that the present disclosure is not entitled to antedate such publication. Further, the dates of publication provided may be different from the actual publication dates which may need to be independently confirmed.

### DETAILED DESCRIPTION

The present disclosure provides therapeutic polypeptides (ICBPs) that comprise: a) a binding moiety comprising a human leukocyte antigen-G (HLA-G) molecule and a stabilizing peptide; b) optionally, at least one immunoinhibitory polypeptide; and c) a scaffold polypeptide. The HLA-G molecule comprises an HLA-G heavy chain polypeptide and a beta-2 microglobulin (32M) polypeptide. The binding moiety of the ICBP binds to immunoglobulin-like transcript 2 (ILT2) and/or immunoglobulin-like transcript 4 (ILT4) polypeptides present on the surface of immune cells (e.g., T cells, B cells, natural killer (NK) cells, monocytes, macrophages, and dendritic cells), while the immunoinhibitory polypeptide binds to a cognate polypeptide (e.g., a receptor or a co-receptor) on the surface of a T-cell. Thus, an ICBP of the present disclosure binds to cells that express both ILT2 and a cognate polypeptide that binds the immunoinhibitory polypeptide or that express both ILT4 and a cognate polypeptide that binds the immunoinhibitory polypeptide. Such cells are generally inflammatory immune cells, such as T cells that exhibit inflammatory activity. In some cases, an ICBP, when administered to an individual in need thereof, suppresses activity of inflammatory immune cells in the individual. In some cases, an ICBP, when administered to an individual in need thereof, increases the number of regulatory T cells (Tregs) in the individual. In some cases, an ICBP, when administered to an individual in need thereof, increases the number of tolerogenic dendritic cells in the individual.

As noted above, an ICBP includes a stabilizing peptide. The stabilizing peptide binds to and stabilizes the HLA-G molecule. However, an ICBP functions to suppress activity of inflammatory immune cells in an antigen non-specific manner. As discussed below, the stabilizing peptide of the binding moiety does not contribute to the binding between the binding moiety and ILT2 or ILT4 receptors. Because an ICBP of this disclosure functions in an antigen non-specific manner, such an ICBP is useful for, e.g., treating an autoimmune disease, including autoimmune diseases that may involve immune cells that are reactive toward multiple self epitopes, or other conditions that involve an undesirable immune response, including metabolic disorders.

In some instances, the ICBP does not include at least one immunoinhibitory polypeptide. That is, the ICBP can be “MOD-less”, i.e., it can comprise: a) a binding moiety comprising a human leukocyte antigen-G (HLA-G) molecule and a stabilizing peptide; and b) a scaffold polypeptide. In such cases, immune tolerance is provided substantially through the interaction of the binding moiety with its receptors on T cells, e.g., ILT2 and ILT4.

### Binding Moieties

As noted above, a binding moiety present in an ICBP of the present disclosure comprises an HLA-G molecule and a stabilizing peptide. An HLA-G molecule comprises an HLA-G heavy chain polypeptide and a β2M polypeptide.

### HLA-G Heavy Chain Polypeptides

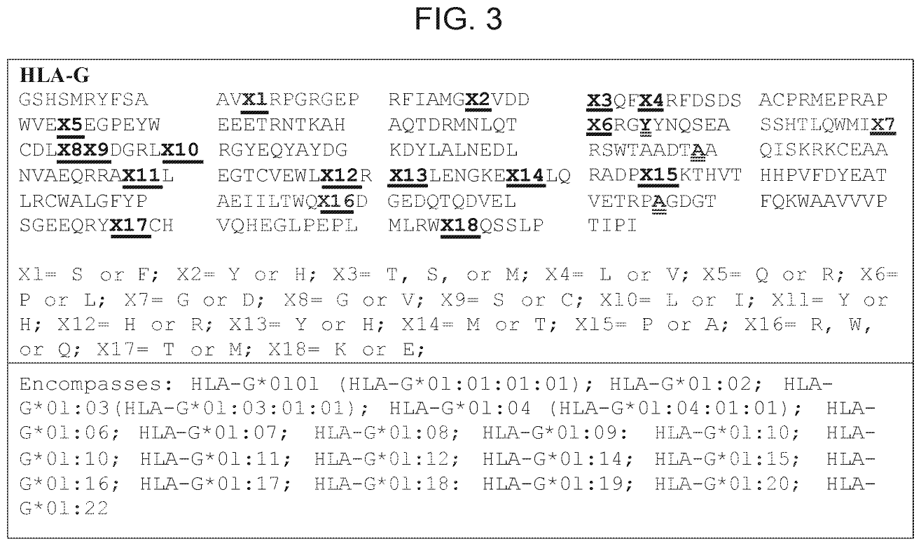

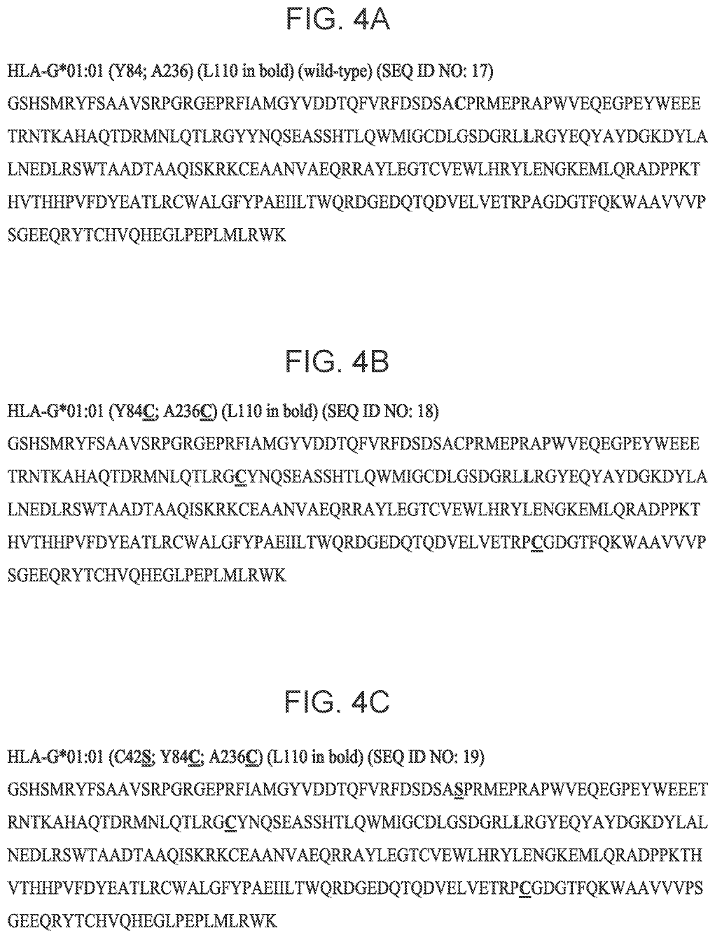

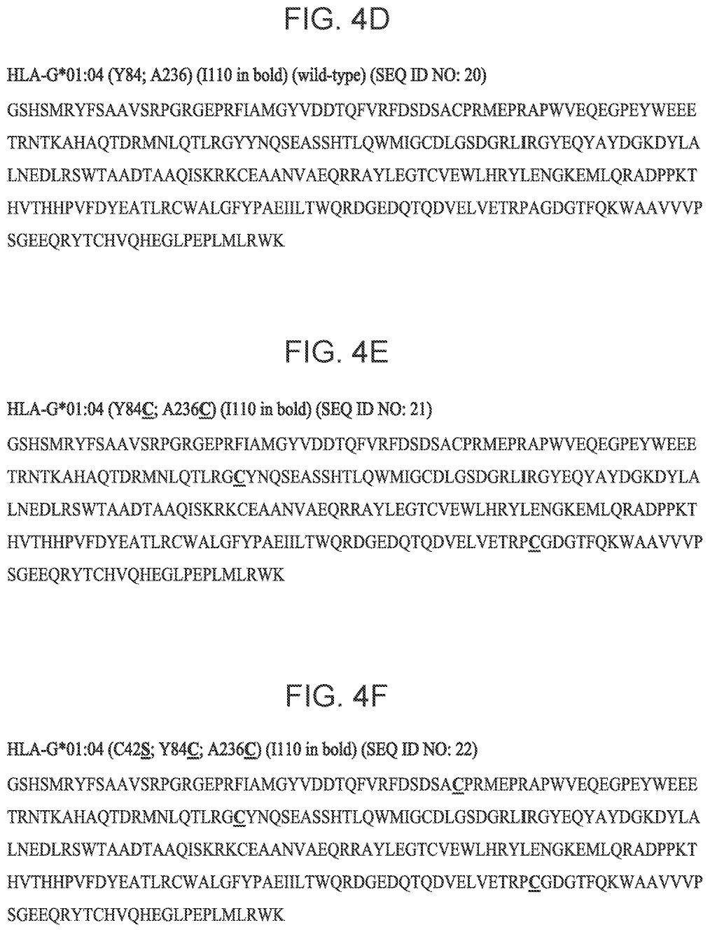

FIG. 3 provides a consensus sequence for HLA-G heavy chain polypeptides with the variable aa positions indicated as “X” residues sequentially numbered and the locations of amino acids (aas) 84, 139 and 236 double underlined. An HLA-G heavy chain polypeptide can comprise an amino acid sequence having at at least 85%, at least 90%, at least 95%, at least 98%, or at least 99%, amino acid sequence identity with the amino acid sequence depicted in any one of FIG. 4A-4F.

In some cases, an HLA-G heavy chain polypeptide present in an ICBP comprises one or more amino acid substitutions relative to a reference HLA-G polypeptide (where a reference HLA-G polypeptide can be a wild-type HLA-G polypeptide). In some cases, an HLA-G heavy chain polypeptide comprises one or more mutation that can preclude homodimerization, e.g., a C42S substitution (see, e.g., Boyson et al. (2002) Proc. Natl. Acad. Sci. USA 99:16180). In some cases, the amino acid substitution substitutes an amino acid with a cysteine (Cys) residue. Such cysteine residues can form a disulfide bond with a naturally occurring or non-naturally occurring cysteine residue present in the HLA-G heavy chain of the ICBP. As used herein, a reference to a “non-naturally occurring Cys residue” in an HLA-G polypeptide means that the polypeptide comprises a Cys residue in a location where there is no Cys in the corresponding wild-type polypeptide. This can be accomplished through routine protein engineering in which a cysteine is substituted for the amino acid that occurs in the wild-type sequence.

Amino acid sequences of suitable HLA-G heavy chain polypeptides are provided in FIG. 4A-4D, where FIG. 4A provides the amino acid sequence of HLA-G*01:01 (wild-type); FIG. 4B provides the amino acid sequence of HLA-G*01:01 with Y84C and A236C substitutions; FIG. 4C provides the amino acid sequence of HLA-G*01:01 with C42S, Y84C, and A236C substitutions; FIG. 4D provides the amino acid sequence of HLA-G*01:04 (wild-type); FIG. 4E provides the amino acid sequence of HLA-G*01:04 with Y84C and A236C substitutions; and FIG. 4F provides the amino acid sequence of HLA-G*01:04 with C42S, Y84C, and A236C substitutions. As discussed below, providing a Cys at residue 84 of the HLA-G heavy chain polypeptide can permit the formation of a disulfide bond with a Cys in the linker joining the stabilizing peptide to the β2M. Providing a Cys at residue 236 of the HLA-G heavy chain polypeptide can permit formation of a disulfide bond that can join the HLA-G heavy chain to a Cys residue in the β2M polypeptide, e.g., at amino acid residue 12 of the β2M polypeptide. Such disulfide bonds can be interchain disulfide bonds (e.g., when the ICBP is a heterodimeric polypeptide) or intrachain disulfide bonds (e.g., when the ICBP is a single-chain polypeptide). In some cases, therefore, an HLA-G heavy chain polypeptide of an ICBP can comprise an amino acid sequence having at least 85%, at least 90%, at least 95%, at least 98%, at least 99%, or 100%, amino acid sequence identity to the amino acid sequence depicted in FIG. 4A, 4B, 4C, 4D, 4E, or 4F. In some cases, an HLA-G heavy chain polypeptide comprises a C42S substitution, in addition to a Y84C substitution and an A236C substitution. In some cases, an HLA-G heavy chain polypeptide of an ICBP can comprise an amino acid sequence having at least 95% or at least 98% amino acid sequence identity to the amino acid sequence depicted in FIG. 4A. In some cases, an HLA-G heavy chain polypeptide of an ICBP can comprise an amino acid sequence having at least 95% or at least 98% amino acid sequence identity to the amino acid sequence depicted in FIG. 4B. In some cases, an HLA-G heavy chain polypeptide of an ICBP can comprise an amino acid sequence having at least 95% or at least 98% amino acid sequence identity to the amino acid sequence depicted in FIG. 4C. In some cases, an HLA-G heavy chain polypeptide of an ICBP can comprise an amino acid sequence having at least 95% or at least 98% amino acid sequence identity to the amino acid sequence depicted in FIG. 4D. In some cases, an HLA-G heavy chain polypeptide of an ICBP can comprise an amino acid sequence having at least 95% or at least 98% amino acid sequence identity to the amino acid sequence depicted in FIG. 4E. In some cases, an HLA-G heavy chain polypeptide of an ICBP can comprise an amino acid sequence having at least 95% or at least 98% amino acid sequence identity to the amino acid sequence depicted in FIG. 4F.

In some cases, an HLA-G heavy chain polypeptide of an ICBP can comprise an amino acid sequence having at least 99% or 100% amino acid sequence identity to the amino acid sequence depicted in FIG. 4A. In some cases, an HLA-G heavy chain polypeptide of an ICBP can comprise an amino acid sequence having at least 99% or 100% amino acid sequence identity to the amino acid sequence depicted in FIG. 4B. In some cases, an HLA-G heavy chain polypeptide of an ICBP can comprise an amino acid sequence having at least 99% or 100% amino acid sequence identity to the amino acid sequence depicted in FIG. 4C. In some cases, an HLA-G heavy chain polypeptide of an ICBP can comprise an amino acid sequence having at least 99% or 100% amino acid sequence identity to the amino acid sequence depicted in FIG. 4D. In some cases, an HLA-G heavy chain polypeptide of an ICBP can comprise an amino acid sequence having at least 99% or at least 100% amino acid sequence identity to the amino acid sequence depicted in FIG. 4E. In some cases, an HLA-G heavy chain polypeptide of an ICBP can comprise an amino acid sequence having at least 99% or 100% amino acid sequence identity to the amino acid sequence depicted in FIG. 4F. An HLA-G heavy chain polypeptide present in an ICBP typically does not include a signal peptide, a transmembrane domain, or a cytoplasmic tail. An HLA-G heavy chain polypeptide present in an ICBP generally includes only the three extracellular domains (α1, α2, and α3 domains). The length of the HLA-G polypeptide can range from about 270 amino acids to about 280 amino acids (e.g., 270 amino acids (aa), 271 aa, 272 aa, 273 aa, 274 aa, 275 aa, 276 aa, 277 aa, 278 aa, 279 aa, or 280 aa). In some cases, the length of the HLA-G polypeptide is 275 amino acids.

### β2M Polypeptides

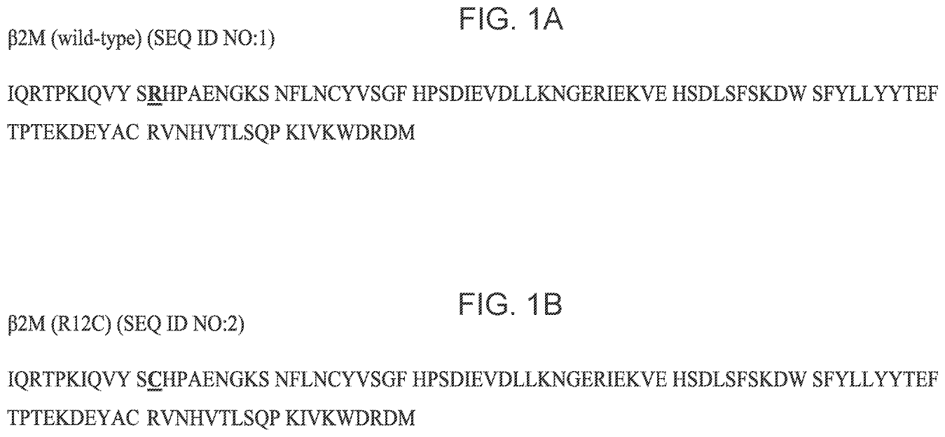

As noted above, an HLA-G molecule includes a β2-microglobulin (β2M) polypeptide. The β2M polypeptide can be a human β2M polypeptide, a non-human primate β2M polypeptide, a murine β2M polypeptide, and the like. In some instances, a β2M polypeptide comprises an amino acid sequence having at least 85%, at least 90%, at least 95%, at least 98%, at least 99%, or 100%, amino acid sequence identity to the amino acid sequence depicted in FIG. 1A (wild-type human β2M). In some cases, a β2M polypeptide present in an ICBP comprises the amino acid sequence: depicted in FIG. 1A (wild-type human β2M). In some instances, a β2M polypeptide comprises an amino acid sequence having at least 90% or at least 95% amino acid sequence identity to the amino acid sequence depicted in FIG. 1A. In some instances, a β2M polypeptide comprises an amino acid sequence having at least 98% or at least 99% amino acid sequence identity to the amino acid sequence depicted in FIG. 1A.

In some cases, a β2M polypeptide present in an ICBP comprises an amino acid sequence having at least 75%, at least 80%, at least 85%, at least 90%, at least 95%, at least 98%, at least 99%, or 100%, amino acid sequence identity to the amino acid sequence depicted in FIG. 1B, where amino acid 12 is a Cys; i.e., where the β2M comprises a non-naturally-occurring Cys at position 12 as a result of an R12C substitution. In some cases, a β2M polypeptide present in an ICBP comprises the amino acid sequence depicted in FIG. 1B. In some instances, a β2M polypeptide comprises an amino acid sequence having at least 90% or at least 95% amino acid sequence identity to the amino acid sequence depicted in FIG. 1B. In some instances, a β2M polypeptide comprises an amino acid sequence having at least 98% or 100% amino acid sequence identity to the amino acid sequence depicted in FIG. 1B.

### Stabilizing Peptides

As noted above, a binding moiety includes a peptide (which may be referred to herein as a “stabilizing peptide”) that binds to the HLA-G molecule. The peptide binds to the peptide binding cleft formed by the α1 and α2 domains of the HLA-G heavy chain polypeptide. The stabilizing peptide can have a length of from about 5 amino acids to about 15 amino acids. For example, the peptide can have a length of 5, 6, 7, 8, 9, 10, 11, 12, 13, 14, or 15 amino acids. In some cases, the stabilizing peptide has a length of from 8 amino acids to 12 amino acids. In some cases, the peptide has a length of from 9 amino acids to 12 amino acids. In some cases, the stabilizing peptide has a length of from 9 amino acids to 11 amino acids. In some cases, the peptide has a length of 9 amino acids.

The binding of the ICBP is not driven by the binding of the stabilizing polypeptide in the binding moiety, but rather by the binding of ILT2 and ILT4 to a complex formed between the α3 domain of the HLA-G heavy chain and the β2M polypeptide. Hence, the peptide presented by the binding moiety does not contribute to the binding between the binding moiety and ILT2 or ILT4 receptors. See, e.g., Attia et al., Int. J. Mol. Sci. 2020, 21(22), 8678; https://doi.org/10.3390/ijms21228678. Accordingly, the origin of the stabilizing peptide, e.g., a self-antigen or a viral antigen, is not important provided that the peptide functionally stabilizes the HLA-G moiety.

In some cases, the stabilizing peptide has the following amino acid sequence: X₁X₂PX₃X₄X₅X₆X₇L, where X₁ is Lys or Arg; X₂ is Gly, Ile, or Leu; X₃ is Ala or Pro; X₄ is Ala, Arg, or Gln; X₅ is Ala, Phe, or Tyr; X₆ is Ile, Len, Val, or Tyr; and X₇ is Ile, Met, Thr, or Gln. In some cases, the stabilizing peptide has the following amino acid sequence: X₁X₂X₃PX₄X₅X₆X₇L, where X₁ is Lys or Arg; X₂ is Gly, Ile, or Leu; X₃ is Ala, Gln, or Ile; X₄ is Ala, Arg, or Gln; X₅ is Ala, Phe, His, or Tyr; X₆ is Ile, Leu, Val, or Tyr; and X₇ is Ile, Met, Thr, or Gln. See, e.g., Diehl et al. (1996) Current Biol. 6:305; and Walpole et al. (2010) J. Mol. Biol. 397:467.

The following are non-limiting examples of stabilizing peptides: RIIPRHLQL (SEQ ID NO:24); RLPKDFRIL (SEQ ID NO:68); KGPPAALTL (SEQ ID NO:69); KLPQAFYIL (SEQ ID NO:70); KLPAQFYIL (SEQ ID NO:71); RIPQGFGNLL (SEQ ID NO:72); KIPAQFYIL (SEQ ID NO:73); KGPAQFYIL (SEQ ID NO:74); RGPPQRPKL (SEQ ID NO:75); RGPPQRPKL (SEQ ID NO:75); RIHDKAVAL (SEQ ID NO:76); RAIQKKIDL (SEQ ID NO:77); RLKKSADTL (SEQ ID NO:78); KSPPPMNL (SEQ ID NO:79); KYIHSANVL (SEQ ID NO:80); KIIDSGPQL (SEQ ID NO:81); TAVISIGNQL (SEQ ID NO:82); VVPKDRVAL (SEQ ID NO:83); RSPVYLTVL (SEQ ID NO:84); RHPKYKTEL (SEQ ID NO:85); HVPEHAVVL (SEQ ID NO:86); MQPTHPIRL (SEQ ID NO:87); KIAGYVTHL (SEQ ID NO:88); KGPPAALTL (SEQ ID NO:69); SYPTRiASL (SEQ ID NO:89); RLPDGRVVL; (SEQ ID NO:90); MRPRKAFLL (SEQ ID NO:91); RLPKDFVDL (SEQ ID NO:92); and VLPKLYVKL (SEQ ID NO:93).

In some cases, a stabilizing peptide is a self-peptide, i.e., a peptide that is associated with a self-antigen. For example, in some cases, the stabilizing peptide presents an autoimmune disease-associated epitope. Numerous epitopes associated with autoimmune disorders autoimmune are known. See, e.g., WO 2020/181062 (Cue Biopharma, Inc.), beginning at paragraph [00386].

In some cases, the stabilizing peptide epitope may be a peptide that presents an epitope associated with an infectious disease such as a viral infection. See, e.g., WO 2020/243315 (Cuc Biopharma, Inc.) beginning at paragraph [00321].

### ILT2 and ILT4

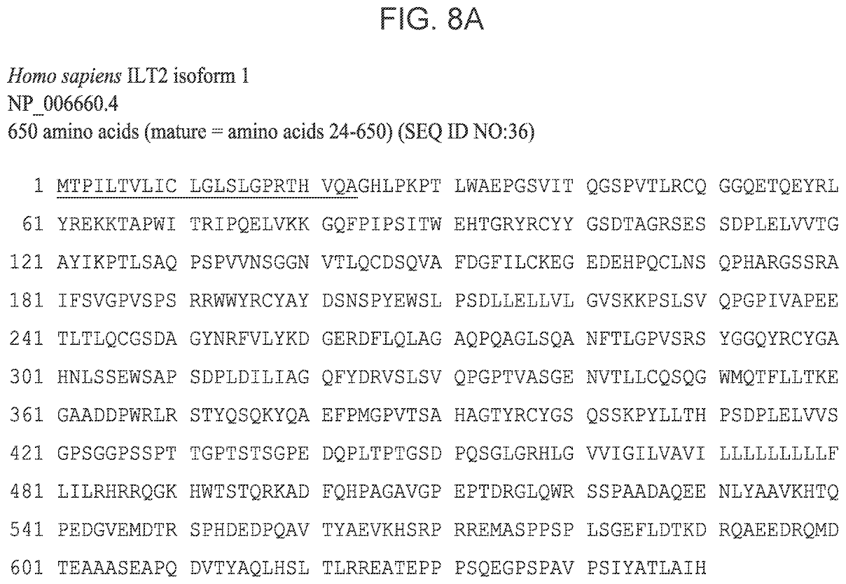

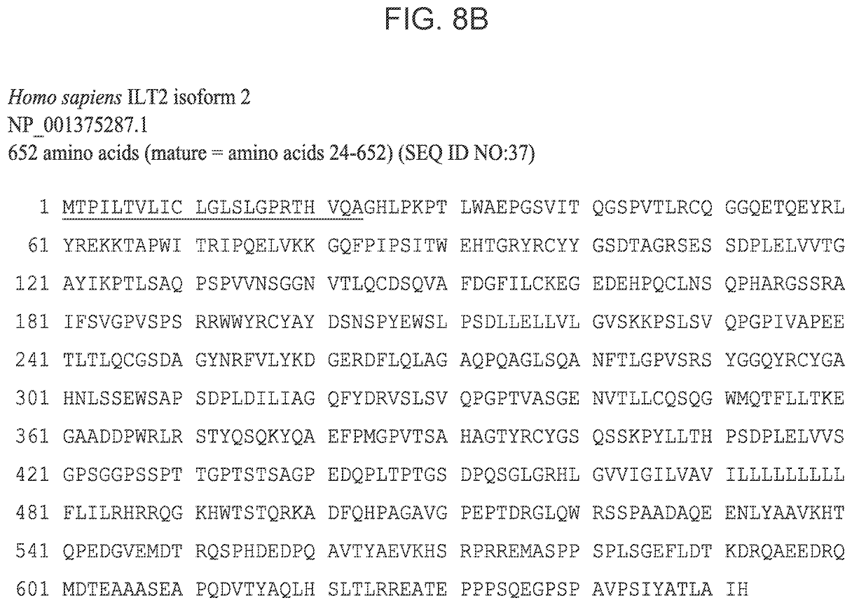

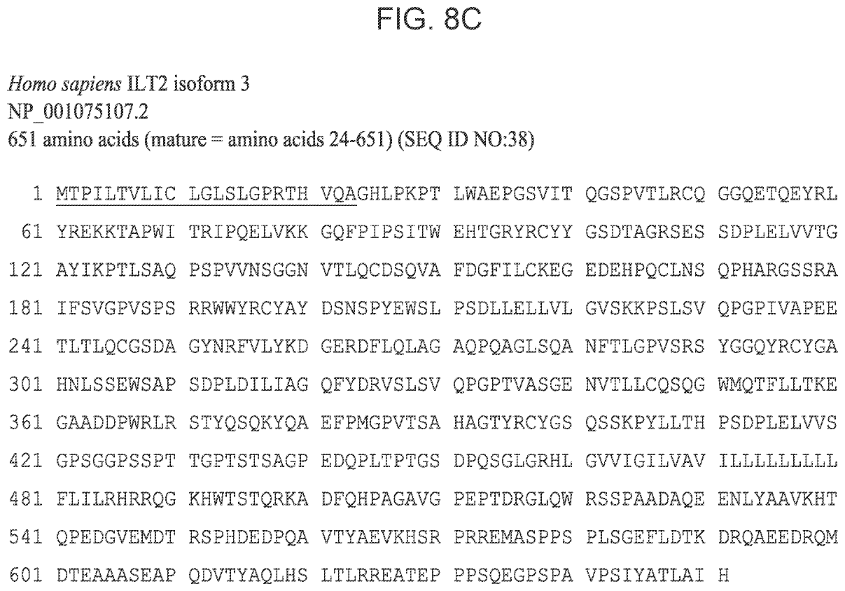

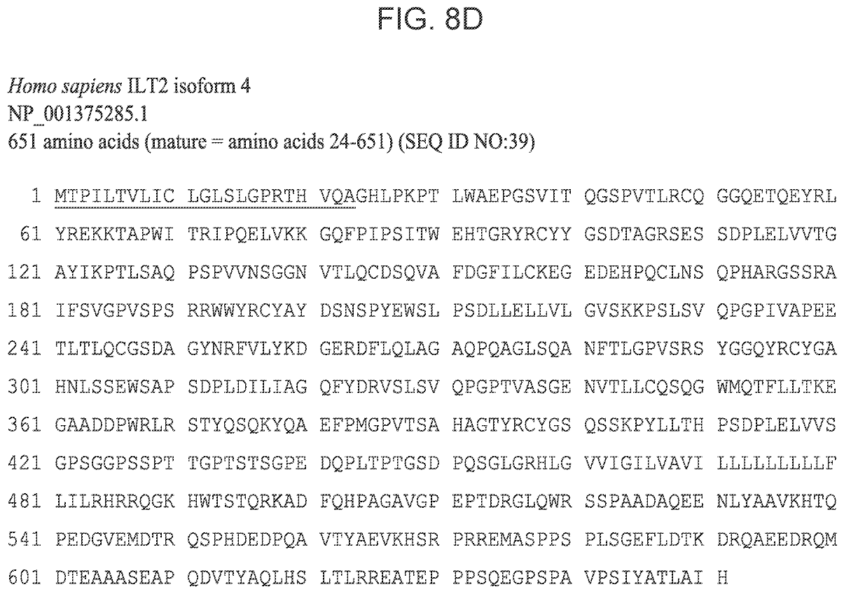

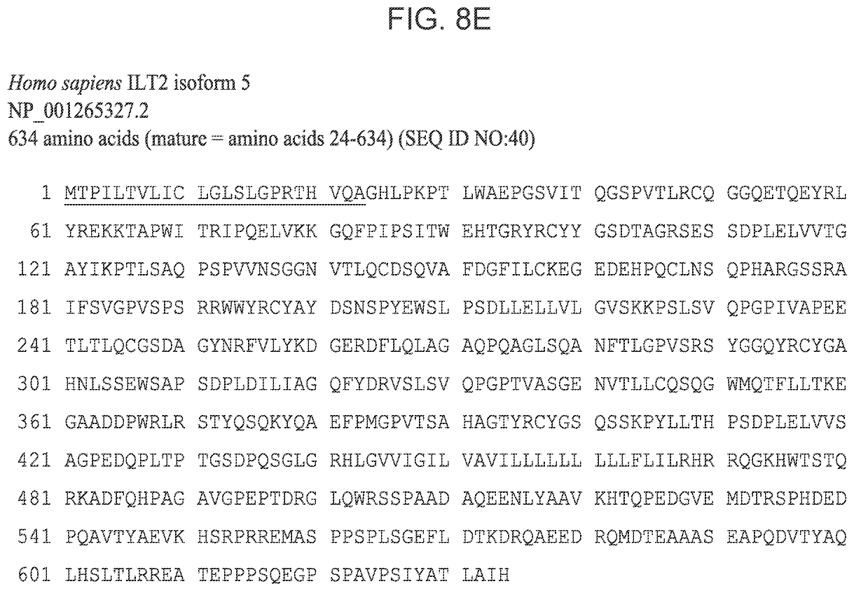

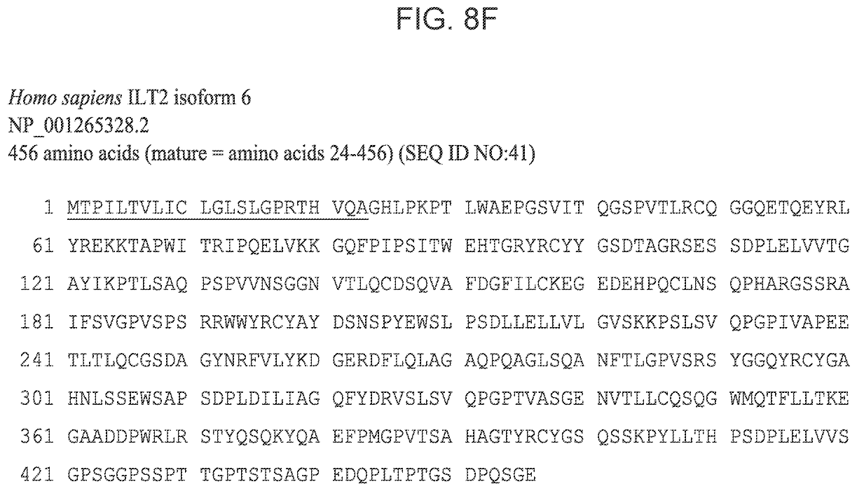

As noted above, the binding moiety of an ICBP of the present disclosure binds to ILT2 and/or ILT4 polypeptides. ILT2 and ILT4 polypeptides are present on the surface of immune cells. ILT2 (also known as “leukocyte immunoglobulin-like receptor B1” (LILRB1), “myeloid inhibitory receptor 7” (MIR-7), and “CD85 antigen-like family member J” (CD85J)) polypeptides are known in the art. ILT2 is expressed on the surface of dendritic cells (DCs), B cells, natural killer (NK) cells, and T cells. Amino acid sequences of ILT2 polypeptides are known in the art. Examples include those depicted in FIG. 8A-8F.

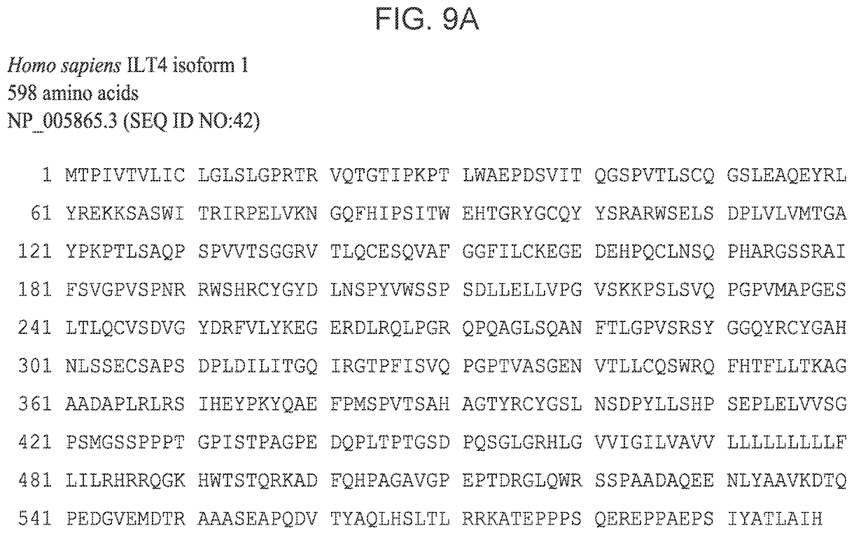

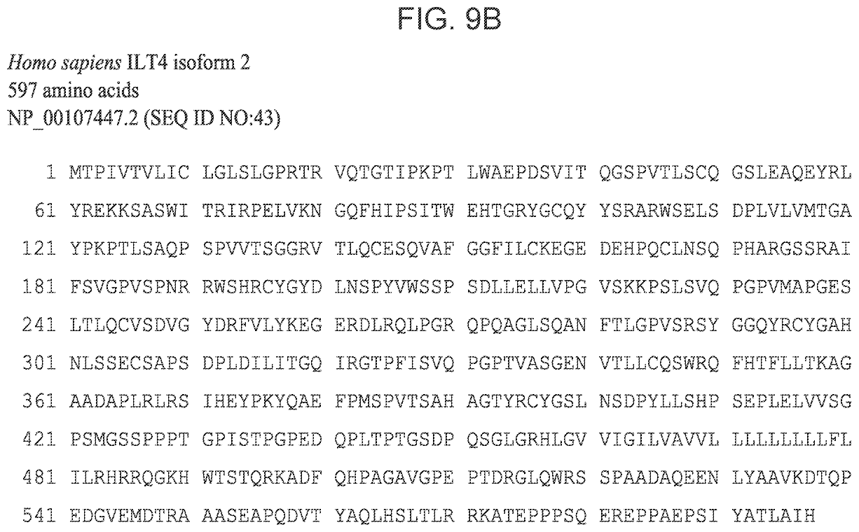

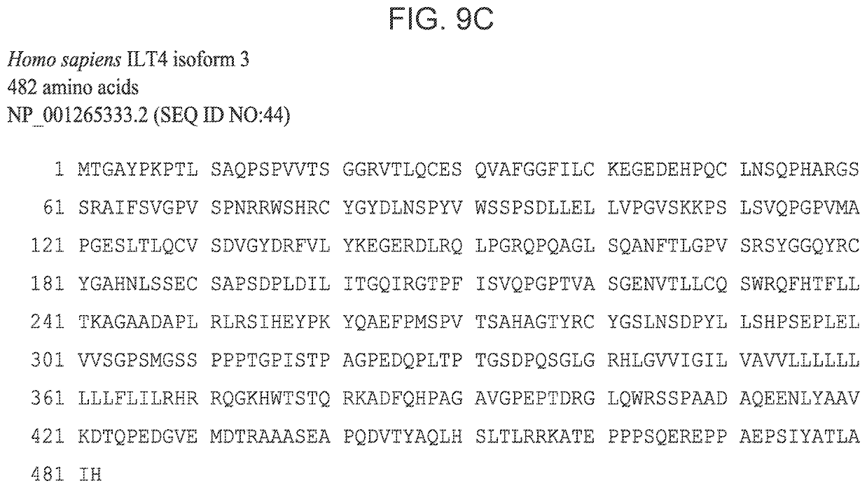

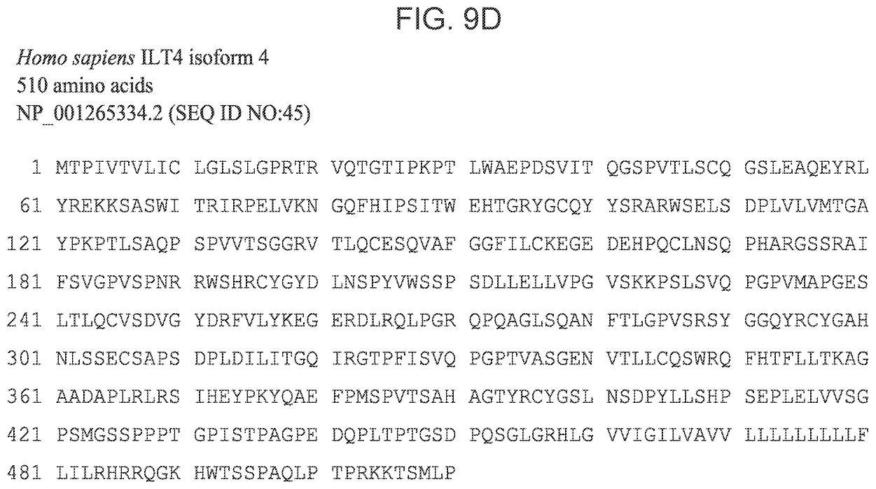

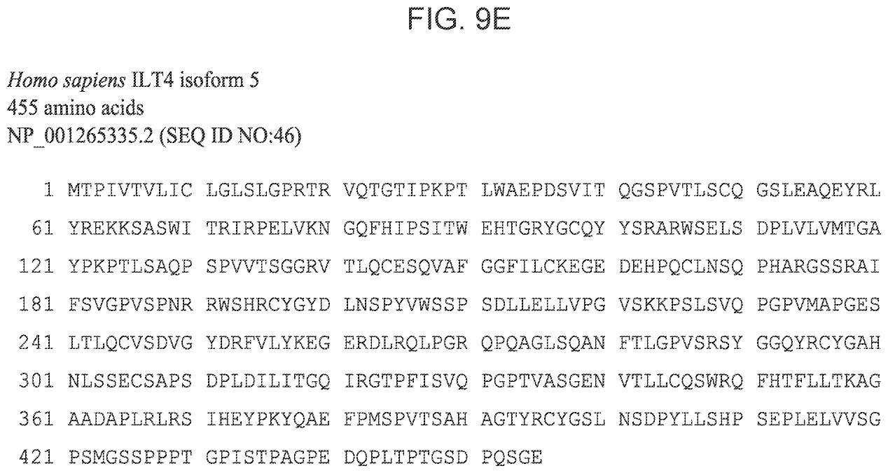

ILT4 (also known as “leukocyte immunoglobulin-like receptor B2” (LILRB2), “myeloid inhibitory receptor 10” (MIR-10), and “CD85 antigen-like family member D” (CD85D)) polypeptides are known in the art. ILT4 is expressed on monocytes and on tolerogenic IL10-producing dendritic cells. Amino acid sequences of ILT4 polypeptides are known in the art. Examples include those depicted in FIG. 9A-9E.

### KIR2DL4

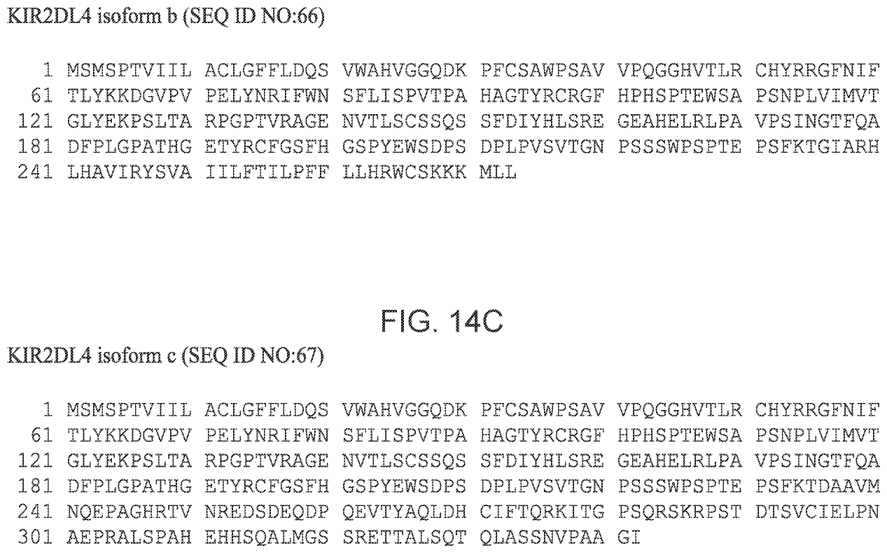

The binding moiety of an ICBP of the present disclosure has been reported to KIR2DL4. See, e.g., Rajagopalan and Long (2012) Front. Immunol. 3:1. KIR2DL4 (“killer cell immunoglobulin like receptor, two Ig domains and long cytoplasmic tail 4”) is also known as CD158D, KIR-103AS, and G9P. KIR2DL4 polypeptides are expressed by natural killer cells and certain T cells. Amino acid sequences of KIR2DL4 polypeptides are known in the art. Examples include those depicted in FIG. 14A-14C.

### Immunoinhibitory Polypeptides

An ICBP of the present disclosure includes one or more immunoinhibitory polypeptides. In some cases, at least one of the one or more immunoinhibitory polypeptides is a wild-type (“wt”) immunoinhibitory polypeptide. In other cases, at least one of the one or more immunoinhibitory polypeptides is a variant of a wt immunoinhibitory polypeptide, e.g., where such a variant has from 1 amino acid (aa) to 20 aa differences from a wt immunoinhibitory polypeptide. In some cases, a variant immunoinhibitory polypeptide comprises one or more conservative amino acid substitutions, compared to a wt immunoinhibitory polypeptide.

In some cases, a variant immunoinhibitory polypeptide binds a cognate co-receptor (co-immunoinhibitory polypeptide) with reduced affinity, compared to the affinity of the binding of a corresponding wt immunoinhibitory polypeptide to the cognate co-receptor. Suitable immunoinhibitory polypeptides that exhibit reduced affinity for a receptor or a co-receptor can have from 1 amino acid (aa) to 20 aa differences from a wild-type immunoinhibitory polypeptide. For example, in some cases, a variant immunoinhibitory polypeptide present in an ICBP differs in amino acid sequence by 1 aa, 2 aa, 3 aa, 4 aa, 5 aa, 6 aa, 7 aa, 8 aa, 9 aa, or 10 aa, from a corresponding wt immunoinhibitory polypeptide. As another example, in some cases, a variant immunoinhibitory polypeptide present in an ICBP differs in amino acid sequence by 11 aa, 12 aa, 13 aa, 14 aa, 15 aa, 16 aa, 17 aa, 18 aa, 19 aa, or 20 aa, from a corresponding wt immunoinhibitory. As an example, in some cases, a variant immunoinhibitory polypeptide present in an ICBP includes 1, 2, 3, 4, 5, 6, 7, 8, 9, or 10 amino acid substitutions, compared to a corresponding reference (e.g., wild-type) immunoinhibitory polypeptide.

An ICBP can comprise a single immunoinhibitory polypeptide. Alternatively, an ICBP can comprise two or more immunoinhibitory polypeptides. For example, an ICBP can comprise two immunoinhibitory polypeptides. The two immunoinhibitory polypeptides can be the same or different (e.g., can have the same amino acid sequence or can have different amino acid sequences). Where an ICBP comprises two immunoinhibitory polypeptides, in some cases, the immunoinhibitory polypeptides are in tandem, optionally separated by a linker (e.g., a peptide linker, as described below).

### PD-L1 Polypeptides

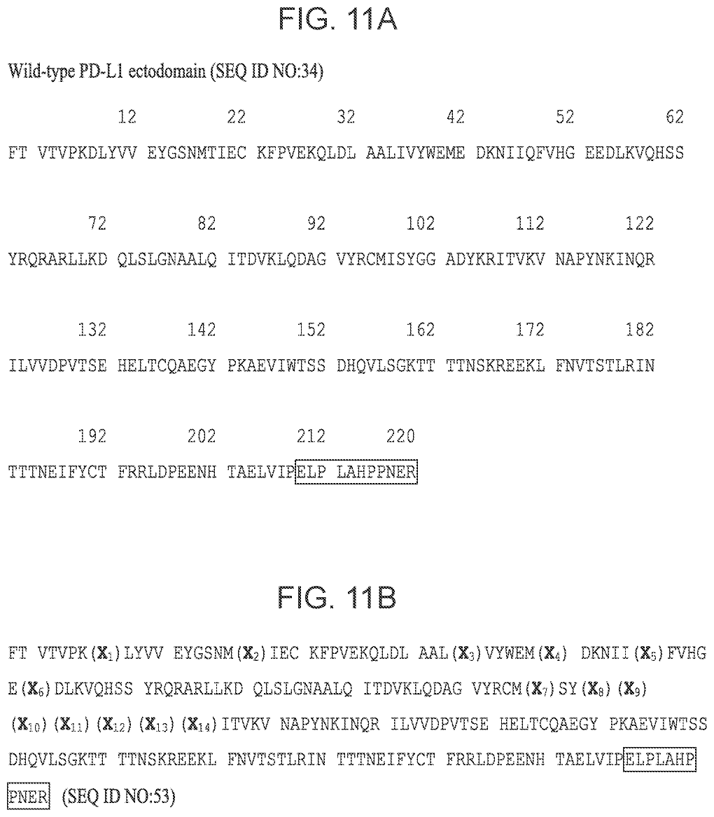

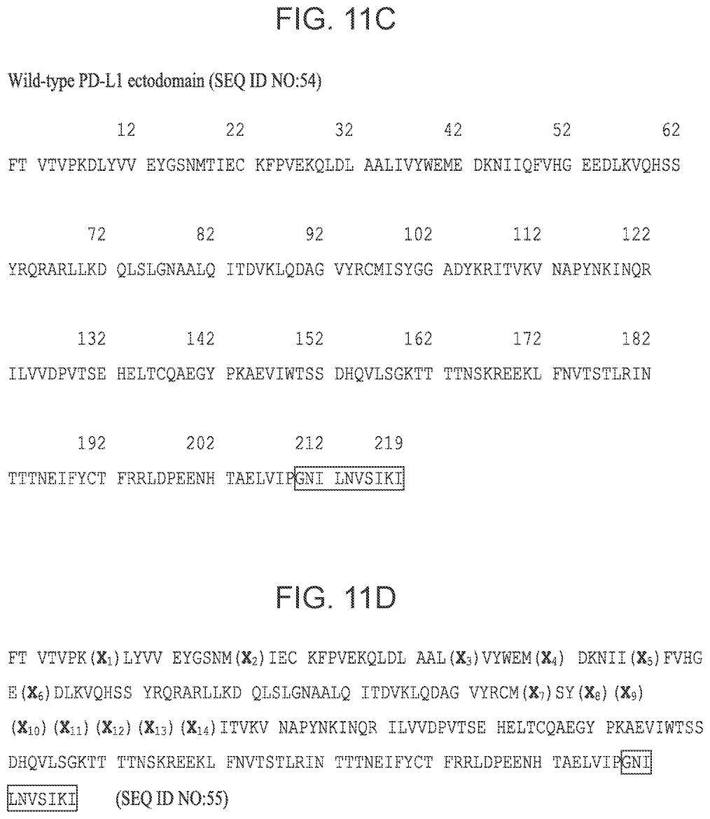

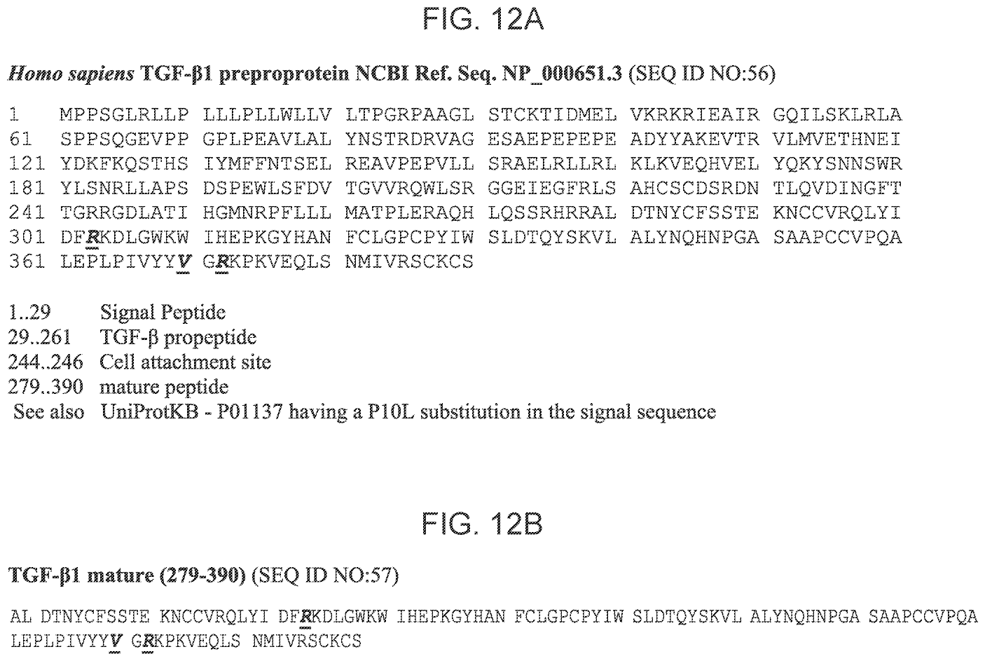

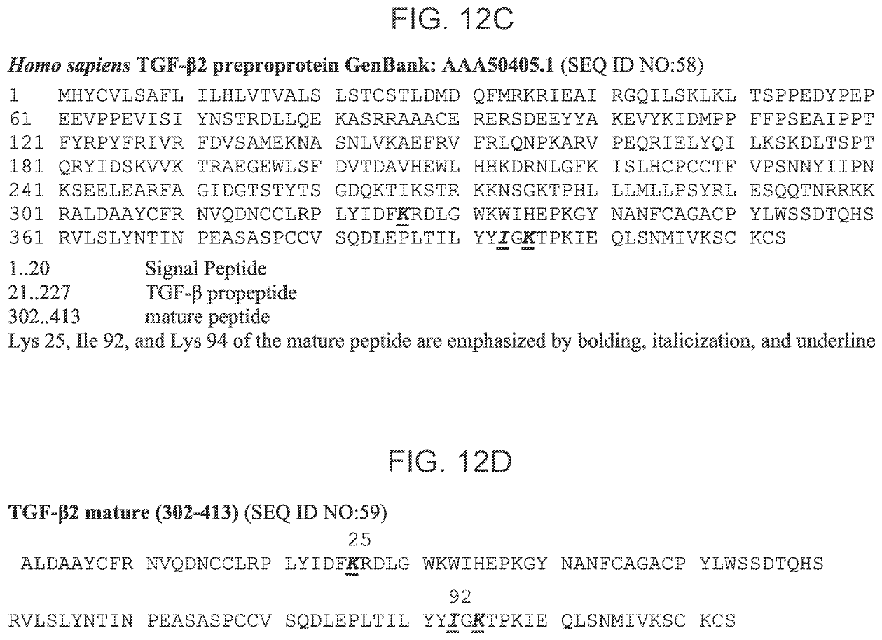

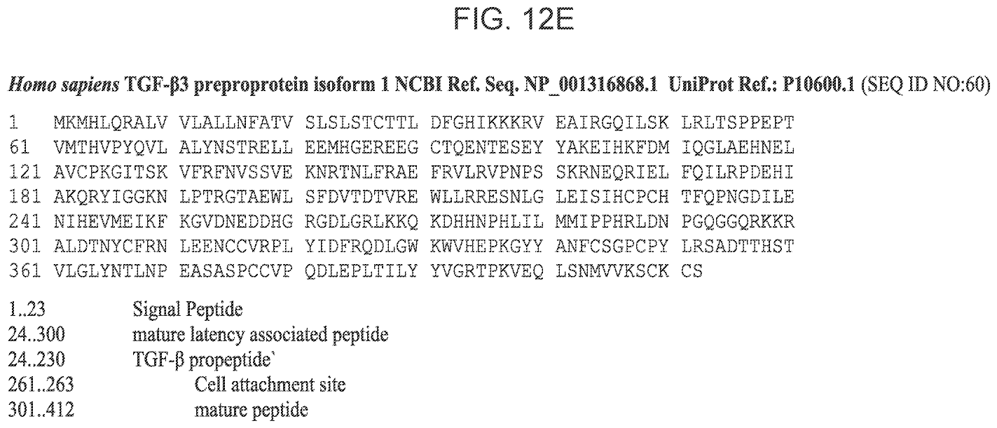

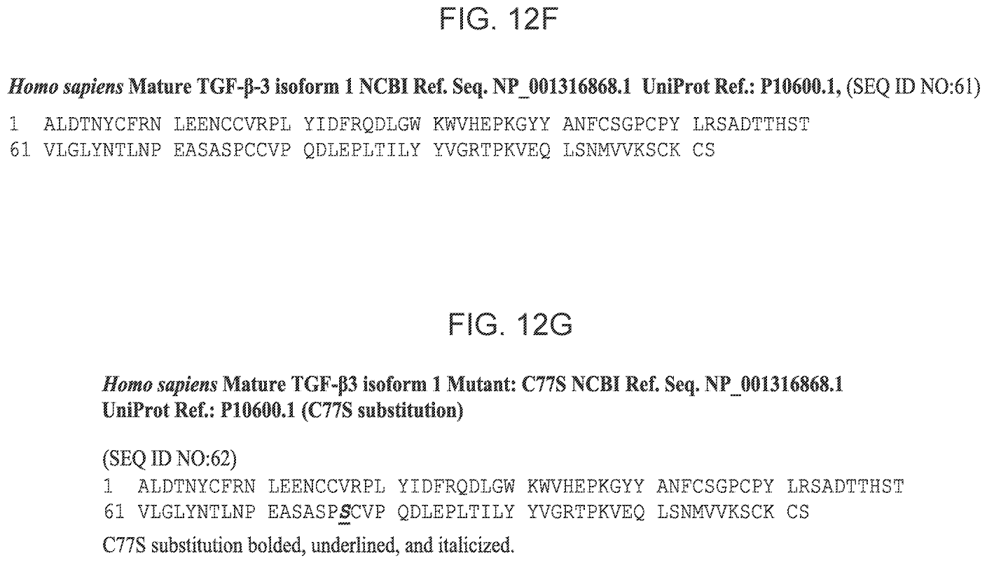

In some cases, an immunoinhibitory polypeptide present in an ICBP is a PD-L1 polypeptide. PD-L1 variants that may be suitable as immunoinhibitory polypeptides are disclosed in Published PCT applications WO 2019/051091 and WO 2017/201131. Amino acid sequences of wild-type and reduced affinity PD-L1 polypeptides are provided in FIG. 11A-11D.

In some cases, a PD-L1 polypeptide of an ICBP comprises an amino acid sequence having at least 85%, at least 90%, at least 95%, at least 98%, at least 99%, or 100%, amino acid sequence identity to the following PD-L1 ectodomain amino acid sequence: FT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKI (SEQ ID NO:54).

In some cases, a PD-L1 polypeptide of an ICBP comprises an amino acid sequence having at least 85%, at least 90%, at least 95%, at least 98%, at least 99%, or 100%, amino acid sequence identity to the following PD-L1 ectodomain amino acid sequence: FT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPELPLAHP PNER (SEQ ID NO:34).

In some cases, a PD-L1 polypeptide comprises an amino acid sequence having at least 85%, at least 90%, at least 95%, at least 98%, or at least 99%, amino acid sequence identity to the following PD-L1 ectodomain amino acid sequence: FT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKI (SEQ ID NO:54); and has a substitution of one or more of the following amino acids: D8, T19, 136, E42, Q48, E54, 198, G101, G102, A103, D104, Y105, K106, and R107.

For example, a PD-L1 polypeptide comprises an amino acid sequence having at least 85%, at least 90%, at least 95%, at least 98%, or at least 99%, amino acid sequence identity to the following PD-L1 ectodomain amino acid sequence: FT VTVPK(X₁)LYVV EYGSNM(X₂)IEC KFPVEKQLDL AAL (X₃)VYWEM(X₄) DKNII(X₅)FVHG E(X₆)DLKVQHSS YRQRARLLKD QLSLGNAALQ ITDVKLQDAG VYRCM(X₇)SY(X₈)(X₉) (X₁₀)(X₁₁) (X₁₂)(X₁₃)(X₁₄)ITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPELPLAHP PNER (SEQ ID NO:53); where X₁ is any amino acid other than Asp, X₂ is any amino acid other than Thr, X₃ is any amino acid other than Ile, X₄ is any amino acid other than Glu, X₅ is any amino acid other than Gln, X₆ is any amino acid other than Glu, X₇ is any amino acid other than Ile, X₈ is any amino acid other than Gly, X₉ is any amino acid other than Gly, X₁₀ is any amino acid other than Ala, X₁₁ is any amino acid other than Asp, X₁₂ is any amino acid other than Tyr, X₁₃ is any amino acid other than Lys, and X₁₄ is any amino acid other than Arg.

As one example, in some cases, a PD-L1 polypeptide comprises an amino acid sequence having at least 85%, at least 90%, at least 95%, at least 98%, or at least 99%, amino acid sequence identity to the following PD-L1 ectodomain amino acid sequence: FT VTVPK(X)LYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPELPLAHP PNER (SEQ ID NO:94), where X is any amino acid other than Asp, e.g., where X is Ala or Arg.

As another example, in some cases, a PD-L1 polypeptide comprises an amino acid sequence having at least 85%, at least 90%, at least 95%, at least 98%, or at least 99%, amino acid sequence identity to the following PD-L1 ectodomain amino acid sequence: FT VTVPKDLYVV EYGSNM(X)IEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPELPLAHP PNER (SEQ ID NO:95), where X is any amino acid other than Thr, e.g., where X is Ala.

As another example, in some cases, a PD-L1 polypeptide comprises an amino acid sequence having at least 85%, at least 90%, at least 95%, at least 98%, or at least 99%, amino acid sequence identity to the following PD-L1 ectodomain amino acid sequence: FT VTVPKDLYVV EYGSNMTIEC KFPVEKQLDL AAL(X)VYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPELPLAHP PNER (SEQ ID NO:96), where X is any amino acid other than Ile, e.g., where X is Asp.

As another example, in some cases, a PD-L1 polypeptide comprises an amino acid sequence having at least 85%, at least 90%, at least 95%, at least 98%, or at least 99%, amino acid sequence identity to the following PD-L1 ectodomain amino acid sequence: FT VTVPKDLYVV EYGSNMTIEC KFPVEKQLDL AALIVYWEM(X) DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPELPLAHP PNER (SEQ ID NO:97), where X is any amino acid other than Glu, e.g., where X is Asp or Arg.

As another example, in some cases, a PD-L1 polypeptide comprises an amino acid sequence having at least 85%, at least 90%, at least 95%, at least 98%, or at least 99%, amino acid sequence identity to the following PD-L1 ectodomain amino acid sequence: FT VTVPKDLYVV EYGSNMTIEC KFPVEKQLDL AALIVYWEME DKNII(X)FVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPELPLAHP PNER (SEQ ID NO:98), where X is any amino acid other than Gln, e.g., where X is Asp.

As another example, in some cases, a PD-L1 polypeptide comprises an amino acid sequence having at least 85%, at least 90%, at least 95%, at least 98%, or at least 99%, amino acid sequence identity to the following PD-L1 ectodomain amino acid sequence: FT VTVPKDLYVV EYGSNMTIEC KFPVEKQLDL AALIVYWEME DKNIIQFVHG E(X)DLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPELPLAHP PNER (SEQ ID NO:99), where X is any amino acid other than Glu, e.g., where X is Asp or Arg.

As another example, in some cases, a PD-L1 polypeptide comprises an amino acid sequence having at least 85%, at least 90%, at least 95%, at least 98%, or at least 99%, amino acid sequence identity to the following PD-L1 ectodomain amino acid sequence: FT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCM(X)SYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPELPLAHP PNER (SEQ ID NO:100), where X is any amino acid other than Ile, e.g., where X is Asp or Arg.

As another example, in some cases, a PD-L1 polypeptide comprises an amino acid sequence having at least 85%, at least 90%, at least 95%, at least 98%, or at least 99%, amino acid sequence identity to the following PD-L1 ectodomain amino acid sequence: FT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISY(X)G ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPELPLAHP PNER (SEQ ID NO:101), where X is any amino acid other than Gly, e.g., where X is Ala, Asp, or Arg.

As another example, in some cases, a PD-L1 polypeptide comprises an amino acid sequence having at least 85%, at least 90%, at least 95%, at least 98%, or at least 99%, amino acid sequence identity to the following PD-L1 ectodomain amino acid sequence: FT VTVPKDLYVV EYGSNMTIEC KFPVEKQLDL AALIVYWEME DKNIIQFVHG EEDLKVQHSS YRQRARLLKD QLSLGNAALQ LTDVKLQDAG VYRCMISYG(X) ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPELPLAHP PNER (SEQ ID NO:102), where X is any amino acid other than Gly, e.g., where X is Ala, Asp, or Arg.

As another example, in some cases, a PD-L1 polypeptide comprises an amino acid sequence having at least 85%, at least 90%, at least 95%, at least 98%, or at least 99%, amino acid sequence identity to the following PD-L1 ectodomain amino acid sequence: FT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG (X)DYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPELPLAHP PNER (SEQ ID NO:103), where X is any amino acid other than Ala, e.g., where X is Arg or Asp.

As another example, in some cases, a PD-L1 polypeptide comprises an amino acid sequence having at least 85%, at least 90%, at least 95%, at least 98%, or at least 99%, amino acid sequence identity to the following PD-L1 ectodomain amino acid sequence: FT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG A(X)YKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPELPLAHP PNER (SEQ ID NO:104), where X is any amino acid other than Asp, e.g., where X is Arg or Ala.

As another example, in some cases, a PD-L1 polypeptide comprises an amino acid sequence having at least 85%, at least 90%, at least 95%, at least 98%, or at least 99%, amino acid sequence identity to the following PD-L1 ectodomain amino acid sequence: FT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG AD(X)KRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPELPLAHP PNER (SEQ ID NO:105), where X is any amino acid other than Tyr, e.g., where X is Asp, Arg, or Ala.

As another example, in some cases, a PD-L1 polypeptide comprises an amino acid sequence having at least 85%, at least 90%, at least 95%, at least 98%, or at least 99%, amino acid sequence identity to the following PD-L1 ectodomain amino acid sequence: FT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADY(X)RITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPELPLAHP PNER (SEQ ID NO:106), where X is any amino acid other than Lys, e.g., where X is Ala, Asp, or Arg.

As another example, in some cases, a PD-L1 polypeptide comprises an amino acid sequence having at least 85%, at least 90%, at least 95%, at least 98%, or at least 99%, amino acid sequence identity to the following PD-L1 ectodomain amino acid sequence: FT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYK(X)ITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPELPLAHP PNER (SEQ ID NO:107), where X is any amino acid other than Arg, e.g., where X is Ala or Asp.

For example, a PD-L1 polypeptide comprises an amino acid sequence having at least 85%, at least 90%, at least 95%, at least 98%, or at least 99%, amino acid sequence identity to the following PD-L1 ectodomain amino acid sequence: FT VTVPK(X₁)LYVV EYGSNM(X₂)IEC KFPVEKQLDL AAL(X₃)VYWEM(X₄) DKNII(X₅)FVHG E(X₆)DLKVQHSS YRQRARLLKD QLSLGNAALQ ITDVKLQDAG VYRCM(X₇)SY(X₈)(X₉) (X₁₀)(X₁₁)(X₁₂)(X₁₃) (X₁₄)ITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKI (SEQ ID NO:55); where X₁ is any amino acid other than Asp, X₂ is any amino acid other than Thr, X₃ is any amino acid other than Ile, X₄ is any amino acid other than Glu, X₅ is any amino acid other than Gln, X₆ is any amino acid other than Glu, X₇ is any amino acid other than Ile, X₈ is any amino acid other than Gly, X₉ is any amino acid other than Gly, X₁₀ is any amino acid other than Ala, X₁₁ is any amino acid other than Asp, X₁₂ is any amino acid other than Tyr, X₁₃ is any amino acid other than Lys, and X₁₄ is any amino acid other than Arg.

### FasL Polypeptides

In some cases, an immunoinhibitory polypeptide present in an ICBP is a FasL polypeptide, e.g., the extracellular domain of a FasL polypeptide. In some cases, a FasL polypeptide of an ICBP comprises an amino acid sequence having at least 85%, at least 90%, at least 95%, at least 98%, at least 99%, or 100%, amino acid sequence identity to the following FasL extracellular domain amino acid sequence:

(SEQ ID NO: 108)
QLFHLQKE LAELRESTSQ MHTASSLEKQ IGHPSPPPEK
KELRKVAHLT GKSNSRSMPL EWEDTYGIVL LSGVKYKKGG
LVINETGLYF VYSKVYFRGQ SCNNLPLSHK VYMRNSKYPQ
DLVMMEGKMM SYCTTGQMWA RSSYLGAVFN LTSADHLYVN
VSELSLVNFE ESQTFFGLYK L.

### TIGIT Ligands

As another example, in some cases, an immunoinhibitory polypeptide present in an ICBP is a TIGIT ligand. TIGIT (“T cell immunoreceptor with Ig and ITIM domains”) is also known as “V-set and immunoglobulin domain-containing protein 9” (VSIG9), “V-set and transmembrane domain-containing protein 3” (VSTM3). TIGIT is expressed on NK cells and T cells (including CD4⁺ T cells, CD8⁺ T cells, and regulatory T cells (Tregs). In some cases, the TIGIT ligand is CD155. CD155 (also known as “poliovirus receptor” (PVR) and “nectin-like protein 5” (Necl-5)) mediates NK cell adhesion and triggers NK cell effector functions. In other instances, the TIGIT ligand is CD112. CD112 (also known as “poliovirus receptor-like 2 (PVRL2), “Herpes virus entry mediator B” (HVEB), and “nectin-2) is a modulator of T-cell signalling.

In some cases, a CD155 polypeptide suitable for inclusion in an ICBP does not include a transmembrane domain present in wild-type CD155; instead, the CD155 polypeptide may include only the extracellular portion of a CD155 polypeptide. For example, the beta and the gamma isoforms of CD155 lack an internal segment including the putative transmembrane domain, compared to the alpha isoform.

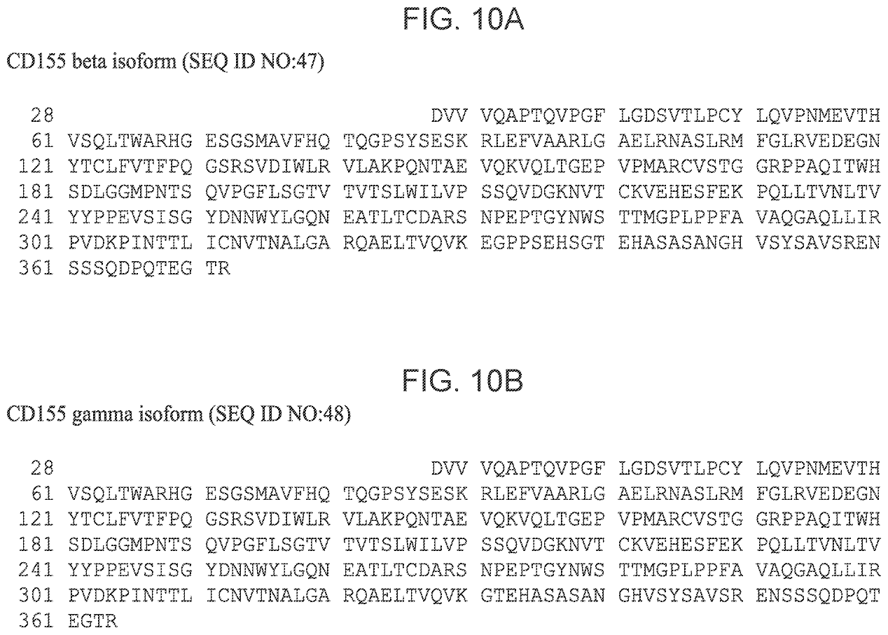

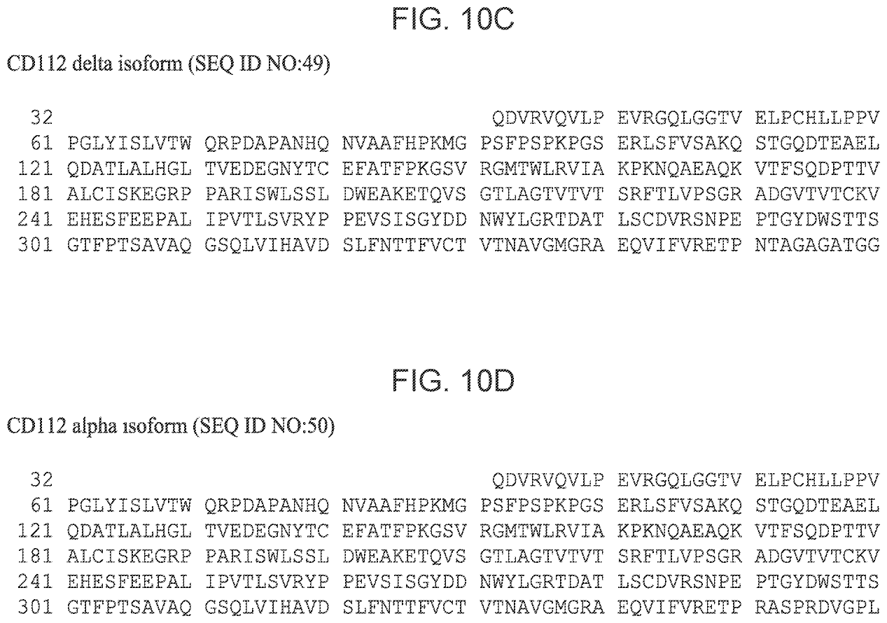

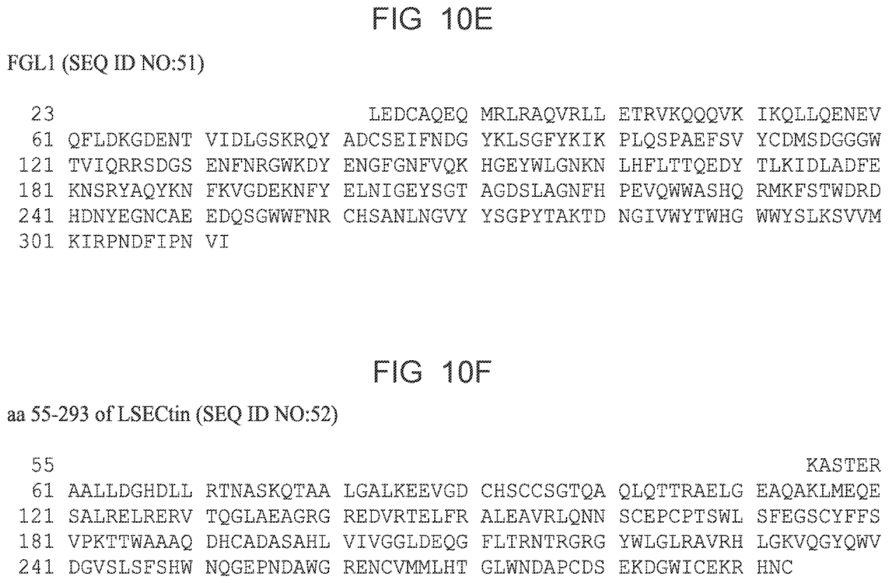

In some cases, a CD155 polypeptide suitable for inclusion in an ICBP comprises an amino acid sequence having at least 85%, at least 90%, at least 95%, at least 98%, at least 99%, or 100%, amino acid sequence identity to the CD155 amino acid sequence depicted in FIG. 10A or FIG. 10B.

In some cases, a CD112 polypeptide suitable for inclusion in an ICBP does not include a transmembrane domain present in wild-type CD112; instead, the CD 112 polypeptide may include only the extracellular portion of a CD112 polypeptide. As non-limiting examples, in some cases, a CD112 polypeptide suitable for inclusion in an ICBP comprises an amino acid sequence having at least 85%, at least 90%, at least 95%, at least 98%, at least 99%, or 100%, amino acid sequence identity to the CD112 amino acid sequence depicted in FIG. 10C or FIG. 10D.

### LAG-3 Ligands

In some cases, an immunoinhibitory polypeptide present in an ICBP is a ligand for a LAG-3 (“Lymphocyte activation gene 3”) polypeptide. A LAG-3 polypeptide is also known as CD223. LAG-3 delivers inhibitory signals upon binding to ligands such as fibrinogen-like protein 1 (FGL1). LAG-3 ligands include FGL1, galectin-3 (Gal-3), lymph node sinusoidal endothelial cell C-type lectin (LSECtin), and α-synuclein fibrils.

FGL1 (also known as “hepatocyte-derived fibrinogen-related protein 1” (HFREP1), “liver fibrinogen-related protein 1” (LFIRE-1), and “hepassocin”) inhibits T-cell activation by acting as a ligand for LAG-3. Wang et al. (2019) Cell 176:334.

In some cases, an FGL1 polypeptide suitable for inclusion in an ICBP comprises an amino acid sequence having at least 85%, at least 90%, at least 95%, at least 98%, at least 99%, or 100%, amino acid sequence identity to the FGL1 amino acid sequence depicted in FIG. 10E.

LSECtin (also known as “C-type lectin domain family 4 member G” (CLEC4G)) can negatively regulate T-cell receptor-mediated signalling. In some cases, an LSECtin polypeptide suitable for inclusion in an ICBP includes only the extracellular portion of LSECtin (and not a transmembrane region). The extracellular portion can include amino acids 55-293 of LSECtin; see, e.g., Dominguez-Soto et al. (2007) Blood 109:5337. Thus, in some cases, an LSECtin polypeptide suitable for inclusion in an ICBP comprises an amino acid sequence having at least 85%, at least 90%, at least 95%, at least 98%, at least 99%, or 100%, amino acid sequence identity to the LSECtin amino acid sequence depicted in FIG. 10F.

### IL-10

As another example, in some cases, an immunoinhibitory polypeptide present in an ICBP is an IL-10 polypeptide. IL-10 is also known as “cytokine synthesis inhibitory factor” (CSIF) and “T-cell growth inhibitor factor” (TGIF). In some cases, the IL-10 polypeptide of an ICBP comprises an amino acid sequence having at least 85%, at least 90%, at least 95%, at least 98%, at least 99%, or 100%, amino acid sequence identity to the following IL-10 amino acid sequence SP GQGTQSENSC THFPGNLPNM LRDLRDAFSR VKTFFQMKDQ LDNLLLKESL LEDFKGYLGC QALSEMIQFY LEEVMPQAEN QDPDIKAHVN SLGENLKTLR LRLRRCHRFL PCENKSKAVE QVKNAFNKLQ EKGIYKAMSE FDIFINYIEA YMTMKIRN (SEQ ID NO:109); and has a length of from about 160 amino acids to about 170 amino acids (e.g., about 166 amino acids).

### IL-27

As another example, in some cases, an immunoinhibitory polypeptide present in an ICBP is an IL-27 polypeptide. In some cases, the IL-27 polypeptide of an ICBP comprises an amino acid sequence having at least 85%, at least 90%, at least 95%, at least 98%, at least 99%, or 100%, amino acid sequence identity to the following IL-27 amino acid sequence MGQTAGDLGW RLSLLLLPLL LVQAGVWGFP RPPGRPQLSL QELRREFTVS LHLARKLLAE VRGQAHRFAE SHLPGVNLYL LPLGEQLPDV SLTFQAWRRL SDPERLCFIS TTLQPFHALL GGLGTQGRWT NMERMQLWAM RLDLRDLQRH LRFQVLAAGF NLPEEEEEEE EEEEEERKGL LPGALGSALQ GPAQVSWPQL LSTYRLLHSL ELVLSRAVRE LLLLSKAGHS VWPLGFPTLS PQP (SEQ ID NO:110); and has a length of from about 225 amino acids to about 250 amino acids (e.g., about 243 amino acids).

### IL-35 Polypeptides

In some cases, an immunoinhibitory polypeptide present in an ICBP is an IL-35 polypeptide. IL-35 is a dimeric protein that includes IL-12α and IL-270 chains. Thus, in some cases, an immunoinhibitory polypeptide present in an ICBP includes both IL-12α and IL-270 polypeptides.

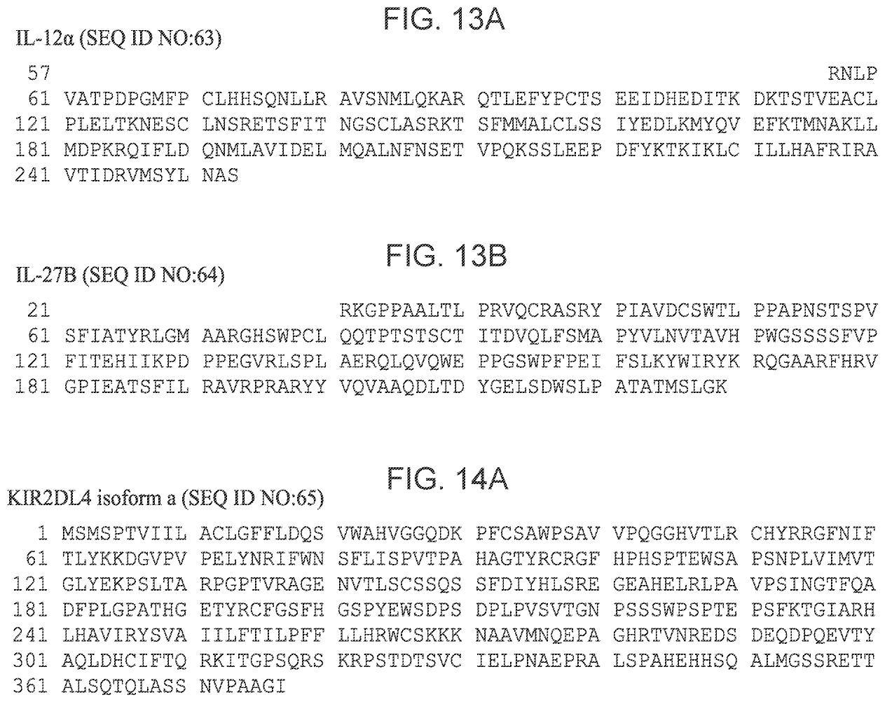

For example, in some cases, IL-12α and IL-27p polypeptides suitable for inclusion in an ICBP comprises an amino acid sequence having at least 85%, at least 90%, at least 95%, at least 98%, at least 99%, or 100%, amino acid sequence identity to the IL-12α and IL-270 amino acid sequence depicted in FIG. 13A and FIG. 13B, respectively.

### IL-2 and TGF-β Polypeptides

As another example, in some cases, an immunoinhibitory polypeptide present in an ICBP is an IL-2 polypeptide. When IL-2 is used an immunoinhibitory polypeptide, typically it will be an IL-2 polypeptide that is not a T cell activating polypeptide but rather a variant of IL-2 that comprises one or more modifications that bias the binding of the variant IL-2 polypeptide to IL-2 Receptor α (IL-2Rα), which can lead to an increase in regulatory T cells (Tregs). Such variants are well known in the art.

Alternatively, however, activating forms of IL-2 may be employed when used in combination with masked TGF-β polypeptides as described in WO 2021/081258 (Cue Biopharma, Inc.).

### Scaffolds

An ICBP includes a scaffold polypeptide. Suitable scaffold polypeptides include antibody-based scaffold polypeptides (e.g., immunoglobulin (Ig) Fc polypeptides) and non-antibody-based scaffolds. Non-antibody-based scaffolds include, e.g., albumin, an XTEN (extended recombinant) polypeptide, transferrin, an Fc receptor polypeptide, an elastin-like polypeptide (see, e.g., Hassouneh et al. (2012) Methods Enzymol. 502:215; e.g., a polypeptide comprising a pentapeptide repeat unit of (Val-Pro-Gly-X-Gly; SEQ ID NO:153), where X is any amino acid other than proline), an albumin-binding polypeptide, a silk-like polypeptide (see, e.g., Valluzzi et al. (2002) Philos Trans R Soc Lond B Biol Sci. 357:165), a silk-elastin-like polypeptide (SELP; see, e.g., Megeed et al. (2002) Adv Drug Deliv Rev. 54:1075), and the like. Suitable XTEN polypeptides include, e.g., those disclosed in WO 2009/023270, WO 2010/091122, WO 2007/103515, US 2010/0189682, and US 2009/0092582; see also Schellenberger et al. (2009) Nat Biotechnol. 27:1186). Suitable albumin polypeptides include, e.g., human serum albumin.

Other suitable scaffold components that can display both an HLA-G binding moiety and an optional immunoinhibitory polypeptide include carriers such as lipid vesicles (e.g., liposomes) or micelles, nanoparticles, PEGylated proteins (including site-specific PEGylation), fibronectin-based scaffold proteins, or artificial antigen presenting cells, such as engineered erythroid cells and enucleated cells (e.g., platelets).

Suitable scaffold polypeptides will in some cases be a half-life extending polypeptides. Thus, in some cases, a suitable scaffold polypeptide increases the in vivo half-life (e.g., the serum half-life) of the ICBP, compared to a control ICBP lacking the scaffold polypeptide. For example, in some cases, a scaffold polypeptide increases the in vivo half-life (e.g., the serum half-life) of the ICBP, compared to a control ICBP lacking the scaffold polypeptide, by at least about 10%, at least about 15%, at least about 20%, at least about 25%, at least about 50%, at least about 2-fold, at least about 2.5-fold, at least about 5-fold, at least about 10-fold, at least about 25-fold, at least about 50-fold, at least about 100-fold, or more than 100-fold. As an example, in some cases, an Fc polypeptide increases the in vivo half-life (e.g., the serum half-life) of the ICBP, compared to a control ICBP lacking the Fc polypeptide, by at least about 10%, at least about 15%, at least about 20%, at least about 25%, at least about 50%, at least about 2-fold, at least about 2.5-fold, at least about 5-fold, at least about 10-fold, at least about 25-fold, at least about 50-fold, at least about 100-fold, or more than 100-fold.

### Ig Fc Polypeptides

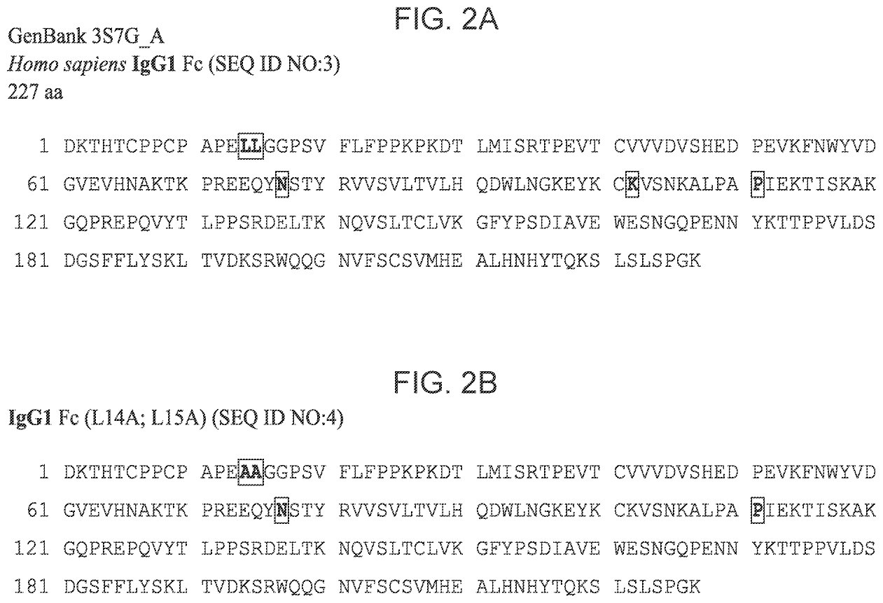

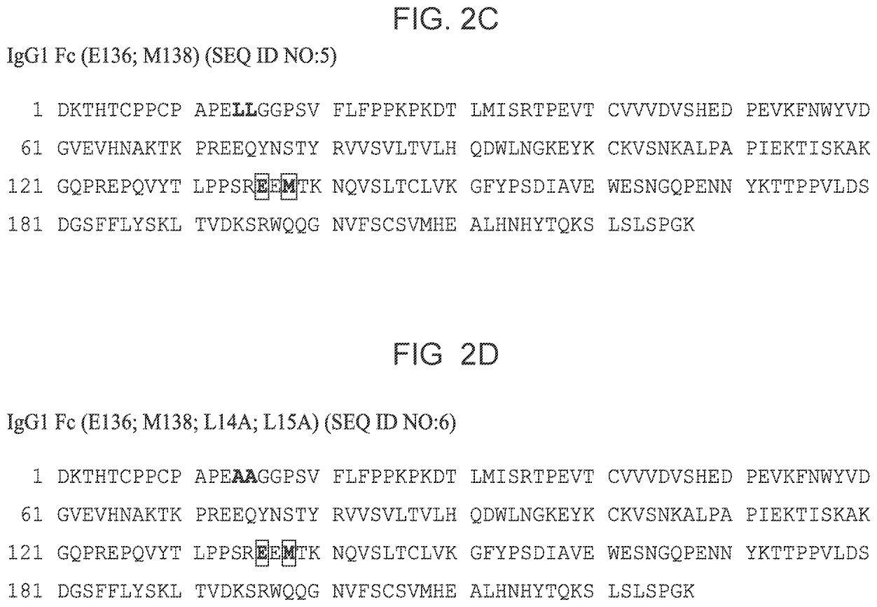

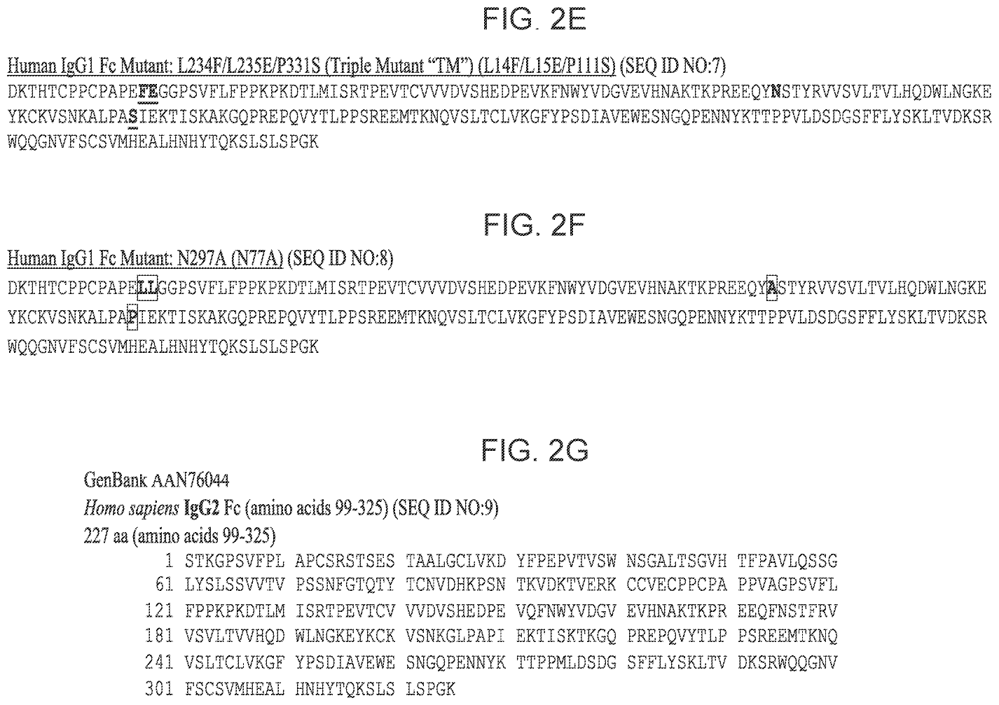

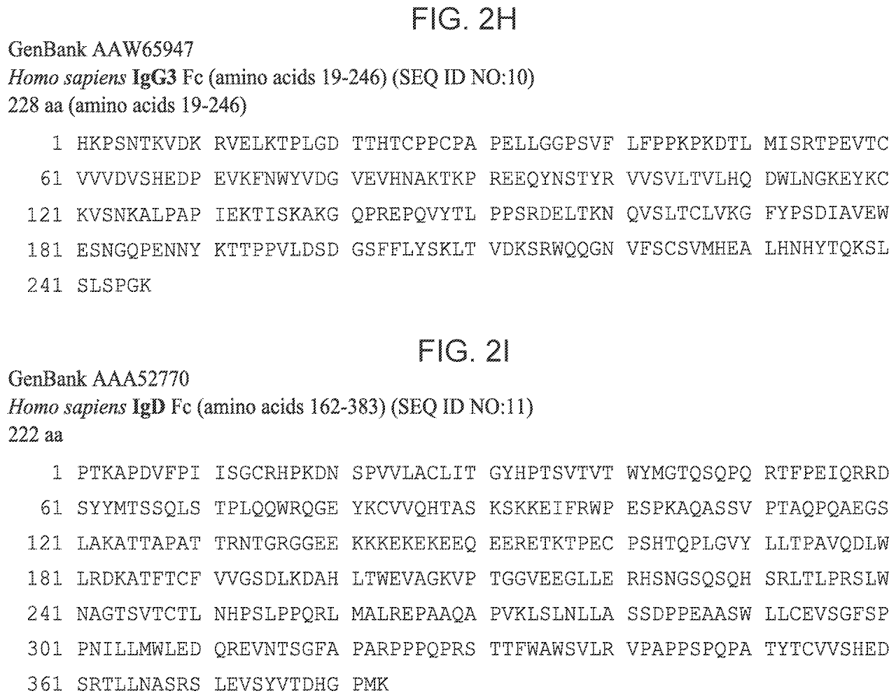

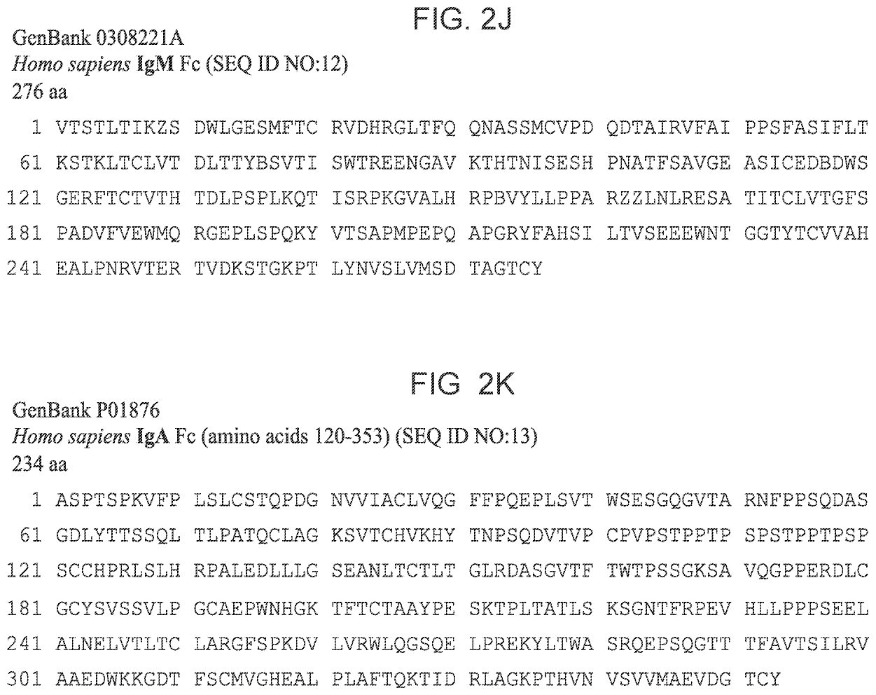

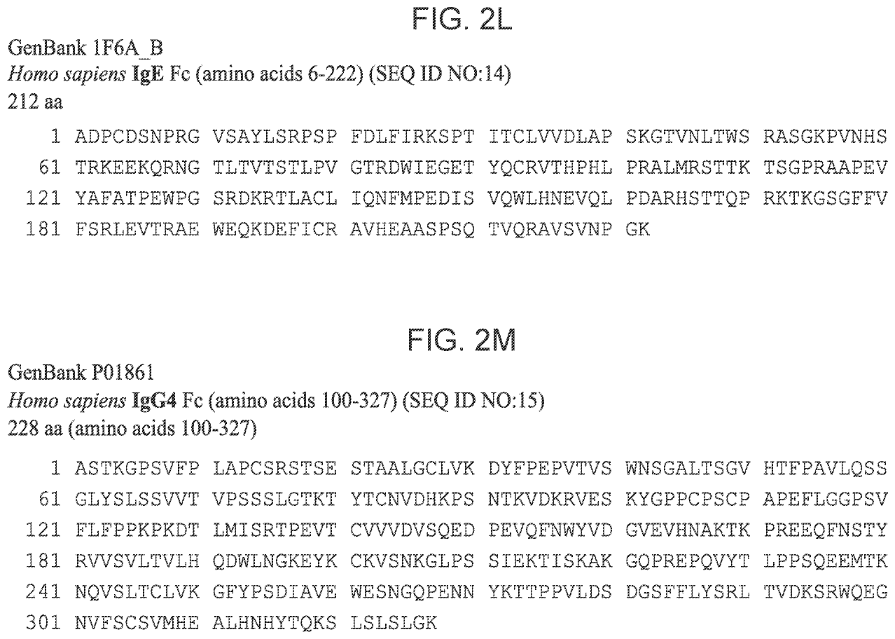

In some cases, a scaffold polypeptide present in an ICBP is an Ig Fc polypeptide. In some cases, an Ig Fc polypeptide in an ICBP comprises an Ig heavy chain constant region (CH2-CH3) polypeptide sequence that functions as a dimerization or multimerization sequence (see, e.g., FIG. 2A-2M). In some cases, the Ig polypeptide will have a reduced ability to induce cell lysis, e.g., through activation of complement-dependent cytotoxicity (CDC) and/or antibody-dependent cellular cytotoxicity (ADCC), and thus may include mutations that reduce or substantially eliminate the ability of the Ig polypeptide to induce cell lysis. In some cases, the Ig Fc polypeptide comprises an amino acid sequence having at least 70%, at least 80%, at least about 90%, at least 95%, at least 98%, at least 99%, or 100% aa sequence identity to an Ig Fc polypeptide depicted in any one of FIG. 2A-2M. Such Ig Fc polypeptides can covalently link the polypeptides of an ICBP together, e.g., by forming one or two interchain disulfide bonds. As discussed below, an additional disulfide bond can be introduced to stabilize dimers, particularly where a pair of interspecific Ig sequence such knob-in-hole (KiH) polypeptide pairs are employed.

In some cases, the Fc polypeptide present in an ICBP comprises an amino acid sequence having at least 85%, at least 90%, at least 95%, at least 98%, at least 99%, or 100%, amino acid sequence identity to the Fc amino acid sequence depicted in any one of FIG. 2A-2M.

In some cases, the Fc polypeptide present in an ICBP is an IgG1 Fc polypeptide, or a variant of an IgG1 Fc polypeptide. For example, in some cases, the Fc polypeptide present in an ICBP comprises an amino acid sequence having at least 85%, at least 90%, at least 95%, at least 98%, at least 99%, or 100%, amino acid sequence identity to the human IgG1 Fc polypeptide depicted in FIG. 2A. As another example, in some cases, the Fc polypeptide present in an ICBP comprises an amino acid sequence having at least 85%, at least 90%, at least 95%, at least 98%, at least 99%, or 100%, amino acid sequence identity to the Fe polypeptide depicted in FIG. 2B, where the Ig Fc polypeptide comprises an Ala at position 14 and an Ala at position 15. In any of the above embodiments, the Ig Fc polypeptide can have an N77 substitution, i.e., the Ig Fc polypeptide can have an amino acid other than Asn at position 77, where in some cases, the Ig Fc polypeptide has an Ala at position 77. In some cases, an Fc polypeptide present in an ICBP comprises the amino acid sequence depicted in FIG. 2A. In some cases, an Fc polypeptide present in an ICBP comprises the amino acid sequence depicted in FIG. 2B.

In some cases, the Fc polypeptide present in an ICBP is an IgG1 Fc polypeptide, or a variant of an IgG1 Fc polypeptide, where variants include naturally occurring variants, non-naturally-occurring variants, and combinations thereof. For example, in some cases, the Fc polypeptide present in an ICBP comprises an amino acid sequence having at least 85%, at least 90%, at least 95%, at least 98%, at least 99%, or 100%, amino acid sequence identity to the human IgG1 Fc polypeptide depicted in FIG. 2C; where the Ig Fc polypeptide comprises a Glu at position 136 and a Met at position 138. As another example, in some cases, the Fc polypeptide present in an ICBP comprises an amino acid sequence having at least 85%, at least 90%, at least 95%, at least 98%, at least 99%, or 100%, amino acid sequence identity to the human IgG1 Fc polypeptide depicted in FIG. 2D; where the Ig Fc polypeptide has Ala at positions 14 and 15; and where the Fc polypeptide comprises a Glu at position 136 and a Met at position 138. In any of the above embodiments, the Ig Fc polypeptide can have an N77 substitution, i.e., the Ig Fc polypeptide can have an amino acid other than Asn at position 77, where in some cases, the Ig Fc polypeptide has an Ala at position 77. In some cases, an Fc polypeptide present in an ICBP comprises the amino acid sequence depicted in FIG. 2C. In some cases, an Fc polypeptide present in an ICBP comprises the amino acid sequence depicted in FIG. 2D.

In some cases, the Fc polypeptide present in an ICBP comprises the amino acid sequence depicted in FIG. 2E (human IgG1 Fc comprising an L234F substitution, an L235E substitution, and a P331S substitution; where L234 corresponds to amino acid 14 of the amino acid sequence depicted in FIG. 2A; L235 corresponds to amino acid 15 of the amino acid sequence depicted in FIG. 2E; and P331 corresponds to amino acid 111 of the amino acid sequence depicted in FIG. 2E). In some cases, the Fc polypeptide present in an ICBP comprises the amino acid sequence depicted in FIG. 2F, comprising an N279A substitution (N77A of the amino acid sequence depicted in FIG. 2F). Substitutions at N297 lead to the removal of carbohydrate modifications and result antibody sequences with reduced complement component 1q (“C1q”) binding compared to the wild-type protein, and accordingly a reduction in complement-dependent cytotoxicity (CDC). In some cases, the Fc polypeptide present in an ICBP comprises a substitution at K322. K322 (e.g., K322A) substitutions shows a substantial reduction in reduction in FcγR binding affinity and a reduction in antibody-dependent cell-mediated cytotoxicity (ADCC), with the C1q binding and CDC functions substantially or completely eliminated.

In some cases, the Fc polypeptide comprises an amino acid sequence having at least 85%, at least 90%, at least 95%, at least 98%, at least 99%, or 100%, amino acid sequence identity to the human IgG2 Fc polypeptide depicted in FIG. 2G; e.g., the Fc polypeptide comprises an amino acid sequence having at least 85%, at least 90%, at least 95%, at least 98%, at least 99%, or 100%, amino acid sequence identity to amino acids 99-325 of the human IgG2 Fc polypeptide depicted in FIG. 2G (e.g., where the Ig Fc polypeptide has a length of about 227 amino acids). In some cases, the Fc polypeptide comprises an amino acid sequence having at least 85%, at least 90%, at least 95%, at least 98%, at least 99%, or 100%, amino acid sequence identity to the human IgG3 Fc polypeptide depicted in FIG. 2H; e.g., the Fc polypeptide comprises an amino acid sequence having at least 85%, at least 90%, at least 95%, at least 98%, at least 99%, or 100%, amino acid sequence identity to amino acids 19-246 of the human IgG3 Fc polypeptide depicted in FIG. 2H (e.g., where the Ig Fc polypeptide has a length of about 228 amino acids). In some cases, the Fc polypeptide comprises an amino acid sequence having at least 85%, at least 90%, at least 95%, at least 98%, at least 99%, or 100%, amino acid sequence identity to the human IgM Fc polypeptide depicted in FIG. 2J; e.g., the Fc polypeptide comprises an amino acid sequence having at least 85%, at least 90%, at least 95%, at least 98%, at least 99%, or 100%, amino acid sequence identity to amino acids 1-276 to the human IgM Fc polypeptide depicted in FIG. 2J. In some cases, the Fc polypeptide comprises an amino acid sequence having at least 85%, at least 90%, at least 95%, at least 98%, at least 99%, or 100%, amino acid sequence identity to the human IgA Fc polypeptide depicted in FIG. 2K; e.g., the Fc polypeptide comprises an amino acid sequence having at least 85%, at least 90%, at least 95%, at least 98%, at least 99%, or 100%, amino acid sequence identity to amino acids 1-234 to the human IgA Fc polypeptide depicted in FIG. 2K.

In some cases, the Fc polypeptide comprises an amino acid sequence having at least 85%, at least 90%, at least 95%, at least 98%, at least 99%, or 100%, amino acid sequence identity to the human IgG4 Fc polypeptide depicted in FIG. 2M. In some cases, the Fc polypeptide comprises an amino acid sequence having at least 85%, at least 90%, at least 95%, at least 98%, at least 99%, or 100%, amino acid sequence identity to amino acids 100 to 327 of the human IgG4 Fc polypeptide depicted in FIG. 2M (e.g., where the Ig Fc polypeptide has a length of about 228 amino acids).

In some cases, the Fc polypeptide present in an ICBP comprises the amino acid sequence depicted in FIG. 2A (human IgG1 Fc). In some cases, the Fc polypeptide present in an ICBP comprises the amino acid sequence depicted in FIG. 2A (human IgG1 Fc), except for a substitution of N297 (N77 of the amino acid sequence depicted in FIG. 2A) with an amino acid other than asparagine (e.g., an N77A substitution). In some cases, the Fc polypeptide present in an ICBP comprises the amino acid sequence depicted in FIG. 2F. Substitutions at N297 (N77) lead to the removal of carbohydrate modifications and result antibody sequences with reduced complement component 1q (“C1q”) binding compared to the wild-type protein, and accordingly a reduction in complement-dependent cytotoxicity (CDC). In some cases, the Fc polypeptide present in an ICBP comprises a substitution at K322 (corresponding to amino acid 102 of the amino acid sequence depicted in FIG. 2A). K322 (e.g., K322A) substitutions shows a substantial reduction in reduction in FcγR binding affinity and a reduction in antibody-dependent cell-mediated cytotoxicity (ADCC), with the C1q binding and CDC functions substantially or completely eliminated. In some cases, the Fc polypeptide present in an ICBP comprises the amino acid sequence depicted in FIG. 2A (human IgG1 Fc), except for a substitution of L234 (L14 of the amino acid sequence depicted in FIG. 2A) with an amino acid other than leucine. In some cases, the Fc polypeptide present in an ICBP comprises the amino acid sequence depicted in FIG. 2A (human IgG1 Fc), except for a substitution of L235 (L15 of the amino acid sequence depicted in FIG. 2A) with an amino acid other than leucine.

Ig Fc heavy chain CH2 and CH3 domains, such as those shown in FIG. 2A-2M, may also function as dimerization or multimerization sequences (e.g., where an ICBP comprises two or more heterodimers). Where an asymmetric pairing between two Ig Fc polypeptides is desired, the Ig Fc polypeptides may incorporate knob-in-hole modifications in, for example the CH3 domain. One such knob-in-hole pair comprises a T366Y and Y407T mutant pair in the CH3 domain interface of IgG1, or the corresponding residues of another Ig Fc (where “T366” corresponds to amino acid 146 of the IgG1 Fc amino acid sequence depicted in FIG. 2A; and “Y407” corresponds to amino acid 187 of the IgG1 Fc amino acid sequence depicted in FIG. 2A). See Ridgway et al., Protein Engineering 9:7, 617-621 (1996), (substitutions are denoted by EU numbering scheme of Kabat et al. (1991)). Another knob-into-hole pairing involves the formation of a knob by a T366W substitution, and a hole by the triple substitutions T366S, L368A and Y407V on the complementary Fc polypeptide (where “T366” corresponds to amino acid 146 of the IgG1 Fc amino acid sequence depicted in FIG. 2A; “L368” corresponds to amino acid 148 of the IgG1 Fc amino acid sequence depicted in FIG. 2A; and “Y407” corresponds to amino acid 187 of the IgG1 Fc amino acid sequence depicted in FIG. 2A). See Xu et al. mAbs 7:1, 231-242 (2015). For example, in some cases, a first ICBP can comprise an IgG1 Fc polypeptide comprising a T366Y substitution (e.g., a T146Y substitution based on the IgG1 Fc amino acid sequence depicted in FIG. 3A); and a second ICBP can comprise an IgG1 Fc polypeptide comprising a Y407T substitution (e.g., a Y187T substitution based on the IgG1 Fc amino acid sequence depicted in FIG. 2A). As another example, in some cases, a first ICBP can comprise an IgG1 Fc polypeptide comprising a T366W substitution (e.g., a T146W substitution based on the IgG1 Fc amino acid sequence depicted in FIG. 2A); and a second ICBP heterodimer can comprise an IgG1 Fc polypeptide comprising a T366S substitution, an L368A substitution, and a Y407V substitution (e.g., a T146S substitution, an L148A substitution, and a Y187V substitution based on the IgG1 Fc amino acid sequence depicted in FIG. 2A). Fc polypeptides, either with or without knob-into-hole modifications, can be stabilized by the formation of disulfide bonds between the Fc polypeptides (e.g., the hinge region disulfide bonds).

### Single-Chain ICBPs

An ICBP of the present disclosure can be a single-chain polypeptide, i.e., all of the components (e.g., HLA-G heavy chain polypeptide, stabilizing peptide, β2M polypeptide, immunoinhibitory polypeptide, scaffold polypeptide) of the ICBP are present in a single polypeptide chain.

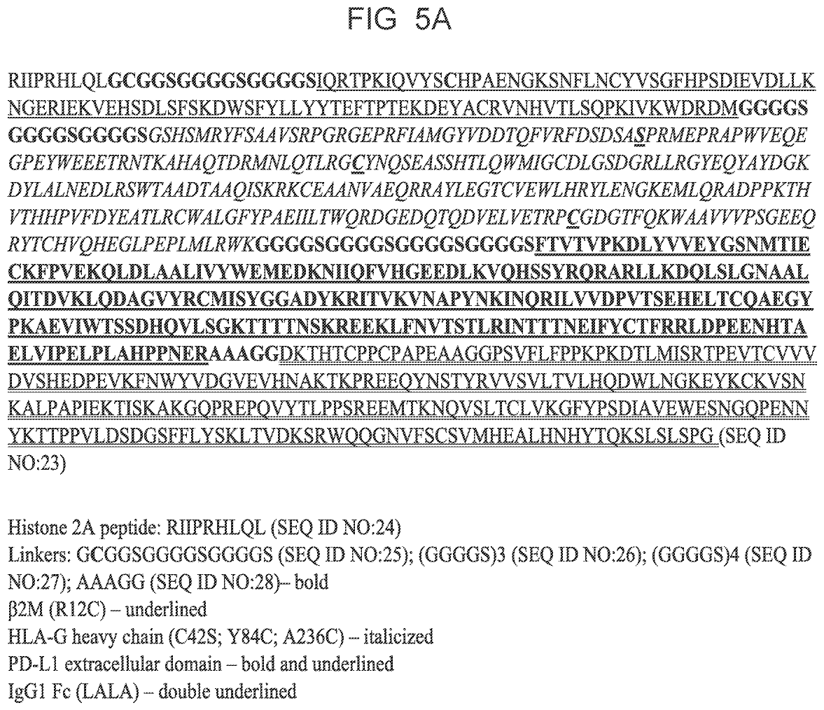

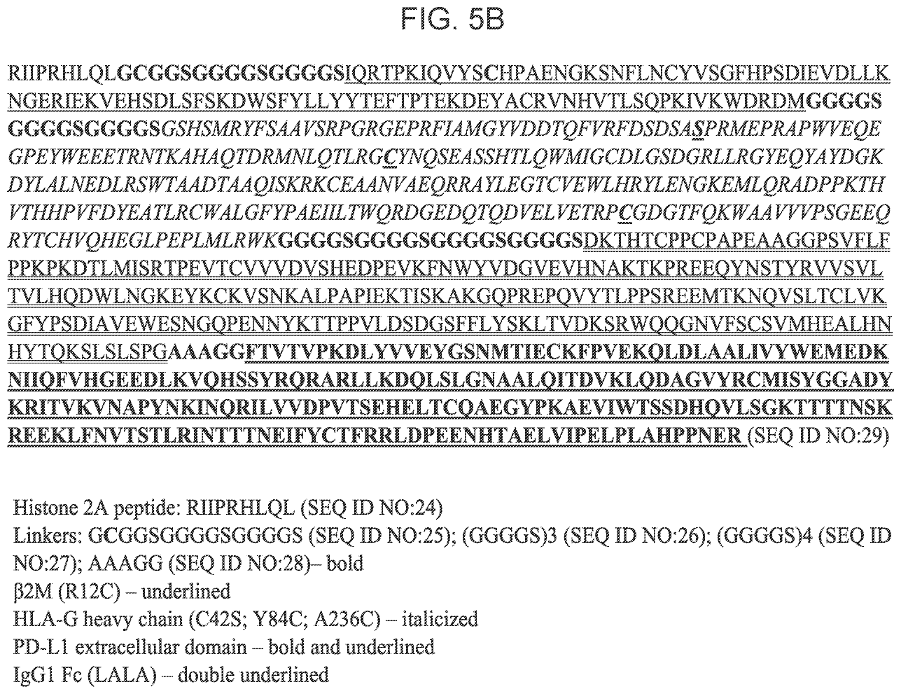

The individual components can be arranged in a variety of configurations. For example, in some cases, an ICBP can comprise, in order from N-terminus to C-terminus: a) a stabilizing peptide; b) a β2M polypeptide; c) an HLA-G heavy chain polypeptide; d) at least one immunoinhibitory polypeptide; and e) a scaffold polypeptide. A non-limiting example of an ICBP having such a configuration is depicted in FIG. 5A. As another example, in some cases, an ICBP can comprise, in order from N-terminus to C-terminus: a) a stabilizing peptide; b) a β2M polypeptide; c) an HLA-G heavy chain polypeptide; d) a scaffold polypeptide; and e) at least one immunoinhibitory polypeptide. A non-limiting example of an ICBP having such a configuration is depicted in FIG. 5B. As noted above, in some cases an ICBP does not comprise at least one immunoinhibitory polypeptide. In such cases, e.g., an ICBP can comprise, in order from N-terminus to C-terminus: a) a stabilizing peptide; b) a β2M polypeptide; c) an HLA-G heavy chain polypeptide; and d) a scaffold polypeptide. As another example, in some cases, an ICBP can comprise, in order from N-terminus to C-terminus: a) a stabilizing peptide; b) a β2M polypeptide; c) an HLA-G heavy chain polypeptide; and d) a scaffold polypeptide. In any of the above embodiments, one or more of the individual components (e.g., HLA-G heavy chain polypeptide, stabilizing peptide, β2M polypeptide, immunoinhibitory polypeptide, scaffold polypeptide) are linked together by one or more linkers (e.g., peptide linkers, as described elsewhere herein). As an example, in some cases, an ICBP can comprise, in order from N-terminus to C-terminus: a) a stabilizing peptide; b) a linker; c) a β2M polypeptide; d) a linker; e) an HLA-G heavy chain polypeptide; f) a linker; g) at least one immunoinhibitory polypeptide; h) a linker; and i) a scaffold polypeptide, e.g., a variant IgG1 Fc polypeptide. As another example, in some cases, an ICBP can comprise, in order from N-terminus to C-terminus: a) a stabilizing peptide; b) a linker; c) a β2M polypeptide; d) a linker; e) an HLA-G heavy chain polypeptide; f) a linker; g) a scaffold polypeptide, e.g., a variant IgG1 Fc polypeptide; h) a linker; and i) at least one immunoinhibitory polypeptide. Exemplary single-chain constructs are provided as FIGS. 5A and 5B. Each of these exemplary constructs comprises an IgG1 Fc scaffold polypeptide having L14A and L15A substitutions. Upon expression, these single-chain ICBPs will spontaneously form homodimers, with disulfide bonds joining the IgG1 Fc polypeptide of one ICBP to the IgG1 Fc of the other single-chain ICBP. In some cases, a single-chain ICBP comprises one or more intrachain disulfide bonds. For example, in some cases, a single-chain ICBP comprises: a) a disulfide bond between (i) a Cys at residue 84 of the HLA-G heavy chain polypeptide and (ii) a Cys in the linker joining the stabilizing peptide to the F2M polypeptide; and/or b) a disulfide bond between a Cys at residue 236 of the HLA-G heavy chain polypeptide and a Cys residue in the β2M polypeptide, e.g., at amino acid residue 12 of the β2M polypeptide.

### Heterodimeric ICBPs

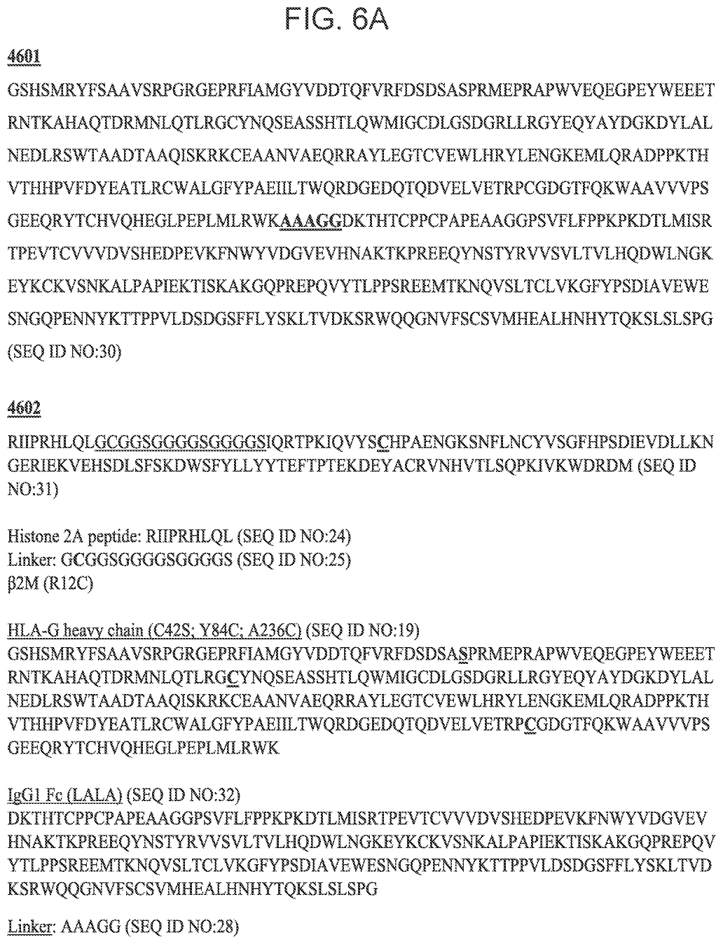

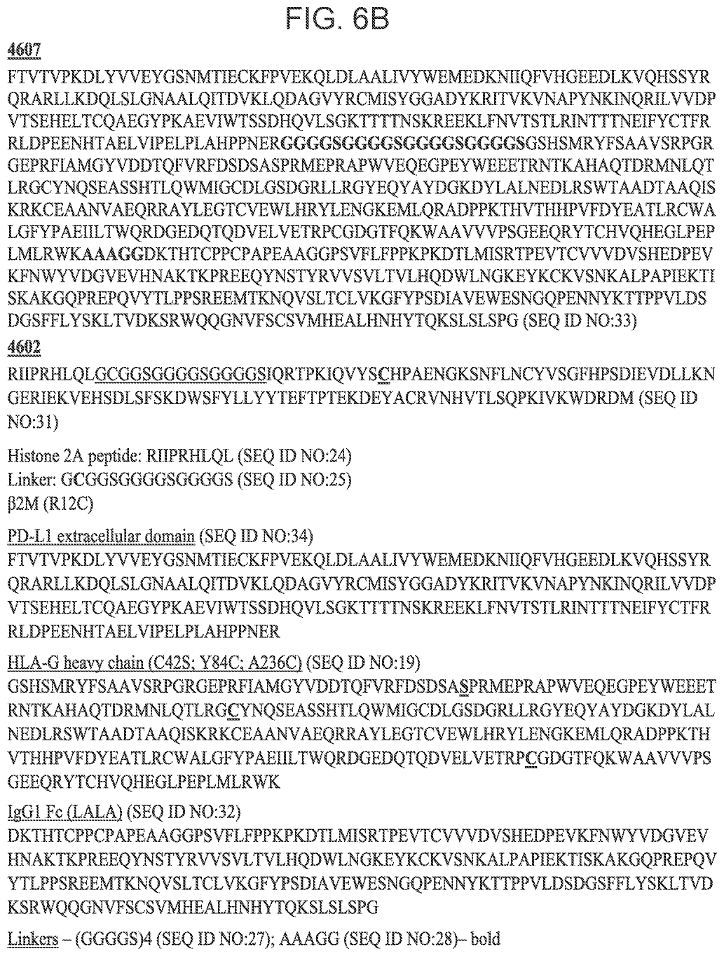

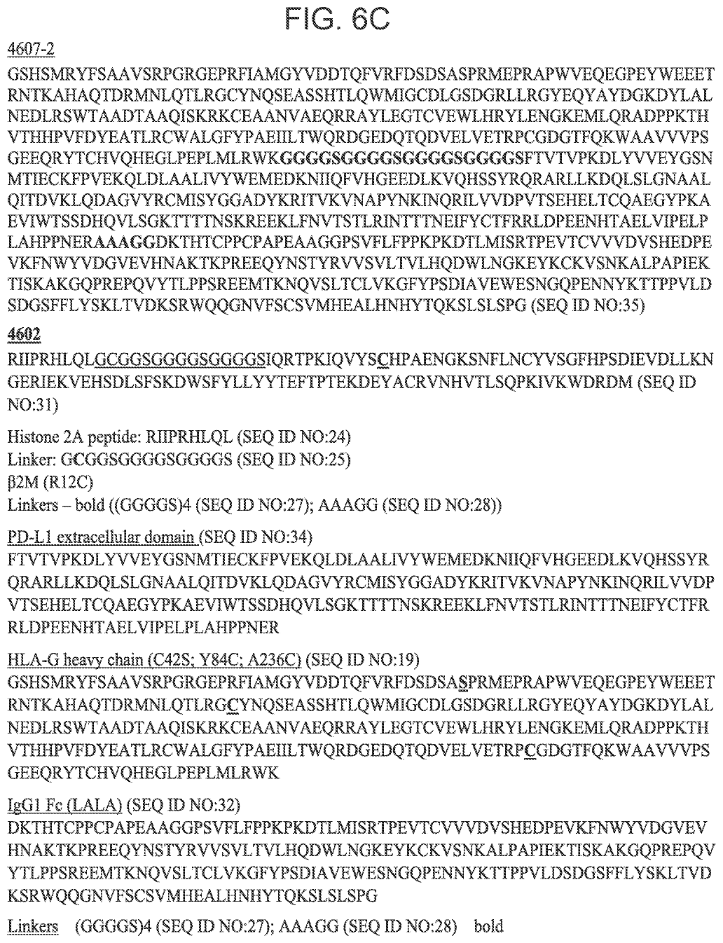

An ICBP of the present disclosure can be a heterodimeric polypeptide; i.e., the components (e.g., HLA-G heavy chain polypeptide, stabilizing peptide, Q2M polypeptide, immunoinhibitory polypeptide, scaffold polypeptide) of the ICBP are present in two separate polypeptide chains. FIG. 6A-6C provide a non-limiting example of polypeptide chains of such a heterodimeric ICBP.

The individual components can be arranged in a variety of configurations. For example, in some cases, an ICBP comprises a first polypeptide comprising a stabilizing peptide and a $2M polypeptide; and a second polypeptide comprising an HLA-G heavy chain polypeptide, at least one immunoinhibitory polypeptide, and a scaffold polypeptide. As another example, in some cases, an ICBP comprises a first polypeptide comprising the binding moiety (i.e., the stabilizing peptide, the HLA-G heavy chain polypeptide, and the β2M polypeptide); and a second polypeptide comprising the at least one immunoinhibitory polypeptide and the scaffold polypeptide.

As an example, in some cases, an ICBP comprises a first polypeptide comprising, in order from N-terminus to C-terminus: (i) a stabilizing peptide; and (ii) a β2M polypeptide; and a second polypeptide comprising, in order from N-terminus to C-terminus: (i) at least one immunoinhibitory polypeptide; (ii) an HLA-G heavy chain polypeptide; and (iii) a scaffold polypeptide. In some cases, the at least one immunoinhibitory polypeptide is a PD-L1 polypeptide (wild-type or reduced-affinity variant). In some cases, the scaffold polypeptide is an Ig Fc polypeptide. FIG. 6B provides examples of such polypeptide chains, where the exemplary first polypeptide chain is designated “4602” and the exemplary second polypeptide chain is designated “4607”. Amino acid sequences of the individual components (e.g., PD-L1 extracellular domain; HLA-G heavy chain polypeptide; and IgG1 Fc) of the second polypeptide chain are also provided. As another example, in some cases, an ICBP comprises a first polypeptide comprising, in order from N-terminus to C-terminus: (i) a stabilizing peptide; and (ii) a β2M polypeptide; and a second polypeptide comprising, in order from N-terminus to C-terminus: (i) an HLA-G heavy chain polypeptide; (ii) at least one immunoinhibitory polypeptide; and (iii) a scaffold polypeptide. In some cases, the at least one immunoinhibitory polypeptide is a PD-L1 polypeptide (wild-type or reduced-affinity variant). In some cases, the scaffold polypeptide is an Ig Fc polypeptide. FIG. 6C provides examples of such polypeptide chains, where the exemplary first polypeptide chain is designated “4602” and the exemplary second polypeptide chain is designated “4607-2”. Amino acid sequences of the individual components (e.g., PD-L1 extracellular domain; HLA-G heavy chain polypeptide; and IgG1 Fc) of the second polypeptide chain are also provided. As another example, in some cases, an ICBP comprises a first polypeptide comprising, in order from N-terminus to C-terminus: (i) a stabilizing peptide; and (ii) a β2M polypeptide; and a second polypeptide comprising, in order from N-terminus to C-terminus: (i) an HLA-G heavy chain polypeptide; (ii) a scaffold polypeptide; and (iii) at least one immunoinhibitory polypeptide. In some cases, the at least one immunoinhibitory polypeptide is a PD-L1 polypeptide (wild-type or reduced-affinity variant). In some cases, the scaffold polypeptide is an Ig Fc polypeptide.

As another example, in some cases, an ICBP comprises a first polypeptide comprising, in order from N-terminus to C-terminus: (i) a stabilizing peptide; (ii) a β2M polypeptide; and (iii) at least one immunoinhibitory polypeptide; and a second polypeptide comprising, in order from N-terminus to C-terminus: (i) an HLA-G heavy chain polypeptide; and (ii) a scaffold polypeptide. In some cases, the at least one immunoinhibitory polypeptide is a PD-L1 polypeptide (wild-type or reduced-affinity variant). In some cases, the scaffold polypeptide is an Ig Fc polypeptide.

As noted above, in some cases, an ICBP does not include an immunoinhibitory polypeptide. For example, in some cases, an ICBP comprises: a) a first polypeptide comprising: (i) a stabilizing peptide; and (ii) a β2M polypeptide; and b) a second polypeptide comprising: (i) an HLA-G heavy chain polypeptide; and ii) a scaffold polypeptide. In some cases, the scaffold polypeptide is an Ig Fc polypeptide. A non-limiting example of such an ICBP is presented in FIG. 6A, where the exemplary first polypeptide chain is designated “4602” and the exemplary second polypeptide is designated “4601.” Amino acid sequences of the individual components (e.g., HLA-G heavy chain polypeptide; and IgG1 Fc) of the second polypeptide chain are also provided. In any of the above embodiments, one or more of the individual components (e.g., HLA-G heavy chain polypeptide, stabilizing peptide, β2M polypeptide, immunoinhibitory polypeptide, scaffold polypeptide) are linked together by one or more linkers (e.g., peptide linkers, as described elsewhere herein).

### Dimeric Forms of an ICBP

As noted above, the components (e.g., HLA-G heavy chain polypeptide, stabilizing peptide, β2M polypeptide, immunoinhibitory polypeptide, scaffold polypeptide) of an ICBP of the present disclosure can be in a single polypeptide chain, or can be present in two separate polypeptide chains that form a heterodimer. Whether the components are present in a single polypeptide chain or in two separate polypeptide chains that form a heterodimer, an ICBP of the present disclosure can form a dimer. Thus, the present disclosure provides a protein comprising a dimer of two ICBPs.

In some cases, the two ICBPs are covalently linked to one another. The covalent linkage of the dimer can be one or more disulfide bonds between an Ig Fc polypeptide in the first ICBP (which may be a single-chain ICBP or a heterodimeric ICBP) and an Ig Fc polypeptide in the second ICBP (which may be a single-chain ICBP or a heterodimeric ICBP). When the ICBP comprises an Ig Fe polypeptide, the ICBP typically will self-assemble into a dimer by spontaneously forming disulfide bonds with the IgG1 Fc polypeptide of another ICBP. Thus, e.g., the Ig Fc polypeptides in the first ICBP and the second ICBP can be linked to one another by one or more disulfide bonds. In many cases, the two ICBPs will be identical to one another in amino acid sequence and comprise Ig Fc polypeptides that spontaneously form one or more disulfide bonds, thereby forming a dimerized ICBP that is a homodimer.

Accordingly, the present disclosure provides a protein comprising: a) a first ICBP; and b) a second ICBP, which optionally may be identical to the first ICBP, where the first and second ICBP are covalently linked to one another. The covalent linkage can be a disulfide bond between an Ig Fc polypeptide in the first ICBP and an Ig Fc polypeptide in the second ICBP. In some cases, the first and second ICBPs are single-chain polypeptides. In some cases, the first and second ICBPs are heterodimeric polypeptides.

Alternatively, where it is desired to link two different ICBPs together to form a dimer, interspecific binding sequences can be employed. See, e.g., WO 2021242935 (Cue Biopharma, Inc.).

### Linkers

As noted above, an ICBP can include one or more independently selected peptide linkers, i.e., a linker comprising a contiguous stretch of two or more amino acids, where the one or more linkers are between one or more components (e.g., HLA-G heavy chain polypeptide, stabilizing peptide, β2M polypeptide, immunoinhibitory polypeptide, scaffold polypeptide) of an ICBP. For example, a linker can be between one or more of: (i) a stabilizing peptide and a β2M polypeptide; (ii) a β2M polypeptide and an HLA-G heavy chain polypeptide (in the case of single-chain ICBPs); (iii) an immunoinhibitory polypeptide and an HLA-G heavy chain polypeptide; (iv) an HLA-G heavy chain polypeptide and a scaffold polypeptide (e.g., an Ig Fc polypeptide); (v) an immunoinhibitory polypeptide and a scaffold polypeptide (e.g., an Ig Fc polypeptide); and (vi) a (32M polypeptide and an immunoinhibitory polypeptide. Linkers may be independently selected from (i) flexible peptide linkers, including short flexible peptide linkers, and (ii) rigid peptide linkers.

Suitable linkers (also referred to as “spacers”) can be readily selected and can be of any of a number of suitable lengths, such as from 1 amino acid to 25 amino acids, from 3 amino acids to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids. A suitable linker can be 1, 2, 3, 4, 5, 6, 7, 8, 9, 10, 11, 12, 13, 14, 15, 16, 17, 18, 19, 20, 21, 22, 23, 24, or 25 amino acids in length. In some cases, a linker has a length of from 25 amino acids to 50 amino acids, e.g., from 25 to 30, from 30 to 35, from 35 to 40, from 40 to 45, or from 45 to 50 amino acids in length.

### Flexible Peptide Linkers

Exemplary flexible peptide linkers include glycine polymers (G),, glycine-serine polymers (including, for example, (GS)ₙ, (GSGGS)ₙ (SEQ ID NO:111), (GGGGS)n (SEQ ID NO:112), and (GGGS)ₙ (SEQ ID NO: 113), where n is an integer of at least one and can be an integer from 1 to 10), glycine-alanine polymers, alanine-serine polymers, and other flexible peptide linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see Scheraga, Rev. Computational Chem. 11173-142 (1992)). Exemplary linkers can comprise amino acid sequences including, but not limited to, GGSG (SEQ ID NO:114), GGSGG (SEQ ID NO: 115), GSGSG (SEQ ID NO:116), GSGGG (SEQ ID NO:117), GGGSG (SEQ ID NO:118), GSSSG (SEQ ID NO:119), and the like.

Exemplary flexible peptide linkers include, e.g., (GGGGS)n (SEQ ID NO:112); also referred to as a “G4S” linker), where n is 1, 2, 3, 4, 5, 6, 7, 8, 9, or 10. In some cases, a linker comprises the amino acid sequence (GGGGS)n (SEQ ID NO:112), where n is 1, 2, 3, 4, 5, 6, 7, 8, 9 or 10. In some cases, a linker comprises the amino acid sequence (GGGGS)n (SEQ ID NO:112), where n is 2. In some cases, a linker comprises the amino acid sequence (GGGGS)n (SEQ ID NO:112), where n is 3. In some cases, a linker comprises the amino acid sequence (GGGGS)n (SEQ ID NO:112), where n is 4. In some cases, a linker comprises the amino acid sequence (GGGGS)n (SEQ ID NO:112), where n is 7. In some cases, a linker comprises the amino acid sequence AAAGG (SEQ ID NO:112). Also suitable is a linker having the amino acid sequence AAAGG (SEQ ID NO:28). In an ICBP of this disclosure, the β2M polypeptide can be connected to the HLA-G heavy chain polypeptide by a (GGGGS)n (SEQ ID NO:112) linker, where n is 1, 2, 3, 4, 5, 6, 7, 8, 9, or 10, e.g., where n=3, n=4, or n=7.

As used in this disclosure, a “short flexible peptide linker” means a flexible peptide linker that comprises fewer than 15 amino acids, i.e., from 2-14 amino acids. For example, a short flexible peptide linker can comprise from 2-4, 2-5, or 3-6 amino acids (e.g., a GGS linker as discussed in Example 1), or from 4-8, 5-10 or from 10-14 amino acids. Within this range includes flexible peptide linkers comprising 2, 3, 4, 5, 6, 7, 8, 9, 10, 11, 12, 13 or 14 amino acids.

### Rigid Peptide Linkers

In some cases, a peptide linker is a rigid peptide linker. As used herein, the term “rigid peptide linker” refers to a linker comprising a contiguous stretch of two or more amino acids that effectively separates protein domains by maintaining a substantially fixed distance/spatial separation between the domains, thereby reducing or substantially eliminating unfavorable interactions between such domains. Rigid peptide linkers are known in the art and generally adopt a relatively well-defined conformation when in solution. Rigid peptide linkers include those which have a particular secondary and/or tertiary structure in solution; and are typically of a length sufficient to confer secondary or tertiary structure to the linker. Rigid peptide linkers include peptide linkers rich in proline, and peptide linkers having an inflexible helical structure, such as an α-helical structure. Rigid peptide linkers are described in, for example, Chen et al. (2013) Adv. Drug Deliv. Rev. 65:1357; and Klein et al. (2014) Protein Engineering, Design & Selection 27:325.

Examples of rigid peptide linkers include, e.g., (EAAAK)n (SEQ ID NO:120), A(EAAAK)nA (SEQ ID NO:121), A(EAAAK)nALEA(EAAAK)nA (SEQ ID NO:122), (Lys-Pro)n, (Glu-Pro)n, (Thr-Pro-Arg)n, and (Ala-Pro)n where n is an integer from 1 to 20 (e.g., n is 1, 2, 3, 4, 5, 6, 7, 8, 9, 10, 11, 12, 13, 14, 15, 16, 17, 18, 19, or 20). Non-limiting examples of suitable rigid peptide linkers comprising EAAAK (SEQ ID NO:123) include EAAAK (SEQ ID NO:123), (EAAAK)₂ (SEQ ID NO:124), (EAAAK)₃ (SEQ ID NO:125), A(EAAAK)₄ALEA(EAAAK)₄A (SEQ ID NO:126), and AEAAAKEAAAKA (SEQ ID NO:127). Non-limiting examples of suitable rigid peptide linkers comprising (AP)n include APAP (SEQ ID NO:128; also referred to herein as “(AP)2”); APAPAPAP (SEQ ID NO:129; also referred to herein as “(AP)4”); APAPAPAPAPAP (SEQ ID NO:130; also referred to herein as “(AP)6”); APAPAPAPAPAPAPAP (SEQ ID NO:131; also referred to herein as “(AP)8”); and APAPAPAPAPAPAPAPAPAP (SEQ TD NO:132; also referred to herein as “(AP)10”). Non-limiting examples of suitable rigid peptide linkers comprising (KP)n include KPKP (SEQ ID NO:133; also referred to herein as “(KP)2”); KPKPKPKP (SEQ ID NO:134; also referred to herein as “(KP)4”); KPKPKPKPKPKP (SEQ ID NO:135; also referred to herein as “(KP)6”); KPKPKPKPKPKPKPKP (SEQ ID NO:136; also referred to herein as “(KP)8”); and KPKPKPKPKPKPKPKPKPKP (SEQ ID NO:137; also referred to herein as “(KP)10”). Non-limiting examples of suitable rigid peptide linkers comprising (EP)n include EPEP (SEQ ID NO:138; also referred to herein as “(EP)2”); EPEPEPEP (SEQ ID NO:139; also referred to herein as “(EP)4”); EPEPEPEPEPEP (SEQ ID NO:140; also referred to herein as “(EP)6”); EPEPEPEPEPEPEPEP (SEQ ID NO:141; also referred to herein as “(EP)8”); and EPEPEPEPEPEPEPEPEPEP (SEQ ID NO:142; also referred to herein as “(EP)10”).

### Cysteine-Containing Linkers

In some cases, a heterodimeric or single-chain ICBP comprises a linker between the stabilizing peptide and the β2M polypeptide that includes a cysteine residue that forms an interchain disulfide bond with a cysteine residue in the HLA-G heavy chain polypeptide. For example, in some cases, where an ICBP, or a dimerized ICBP such as a homodimer, comprises a cysteine-containing linker between the stabilizing peptide and the β2M polypeptide, the cysteine residue in the linker forms a disulfide bond with a cysteine residue at amino acid 236 (e.g., formed by an A236C substitution) in the HLA-G heavy chain polypeptide present in the ICBP.

In some cases, the peptide linker between the stabilizing peptide and the β2M polypeptide comprises the amino acid sequence GCGGS (SEQ ID NO:143). In some cases, the peptide linker between the stabilizing peptide and the β2M polypeptide comprises the amino acid sequence GCGGS(GGGGS)n (SEQ ID NO:144), where n is 1, 2, 3, 4, 5, 6, 7, 8, 9, or 10; e.g., 1, 2, or 3. In some cases, the peptide linker between the peptide and the β2M polypeptide comprises the amino acid sequence GCGGS(GGGGS)n (SEQ ID NO:144), where n is 2. In some cases, the peptide linker between the stabilizing peptide and the (32M polypeptide comprises the amino acid sequence CGGGS (SEQ ID NO:145). In some cases, the peptide linker comprises the amino acid sequence CGGGS(GGGGS)n (SEQ ID NO: 146), where n is 1, 2, 3, 4, 5, 6, 7, 8, 9, or 10; e.g., 1, 2, or 3. In some cases, the peptide linker between the stabilizing peptide and the β2M polypeptide comprises the amino acid sequence GGCGS (SEQ ID NO:147). In some cases, the peptide linker comprises the amino acid sequence GGCGS(GGGGS)n (SEQ ID NO:148), where n is 1, 2, 3, 4, 5, 6, 7, 8, 9 or 10, e.g., 1, 2, or 3. In some cases, the peptide linker between the stabilizing peptide and the (32M polypeptide comprises the amino acid sequence GGGCS (SEQ ID NO:149). In some cases, the peptide linker comprises the amino acid sequence GGGCS(GGGGS)n (SEQ ID NO:150), where n is 1, 2, 3, 4, 5, 6, 7, 8, 9, or 10, e.g., 1, 2, or 3. In some cases, the peptide linker between the stabilizing peptide and the β2M polypeptide comprises the amino acid sequence GGGGC (SEQ ID NO:151). In some cases, the peptide linker comprises the amino acid sequence GGGGC(GGGGS)n (SEQ ID NO:152), where n is 1, 2, 3, 4, 5, 6, 7, 8, 9, or 10, e.g., 1, 2, or 3.

### Compositions

The present disclosure provides compositions, including pharmaceutical compositions, comprising an ICBP of the present disclosure.

A wide variety of pharmaceutically acceptable ingredients are known in the art and hence are not discussed in detail herein. Moreover, pharmaceutically acceptable ingredients and compositions have been amply described in a variety of publications, including, but not limited to, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc. Many other publications describing preparation of biopharmaceutical compositions may be consulted.

The composition may be formulated according to the various routes of administration described below. Generally speaking, ICBPs of this disclosure (e.g., a homodimer comprising two ICBPs) will be an aqueous liquid and typically will be administered via an intravenous infusion. In some cases, the pharmaceutical composition comprising the ICBP can be admixed with saline (e.g., 0.9% NaCl) prior to intravenous administration. Thus, the present disclosure provides a sterile composition comprising: a) an ICBP (or a protein comprising two ICBPs); and b) saline (e.g., 0.9% NaCl). Alternatively, an ICBP may be administered neat via an intravenous infusion, i.e., without further dilution. Alternatively, the pharmaceutical composition comprising the ICBP may be formulated so as to be administered by injection.

Conventional and pharmaceutically acceptable routes of administration include intratumoral, peritumoral, intramuscular, intralymphatic, intratracheal, intracranial, subcutaneous, intradermal, topical application, intravenous, intraarterial, rectal, nasal, oral, and other enteral and parenteral routes of administration. As noted above, a pharmaceutical composition comprising an ICBP typically will be administered intravenously, but may also be administered by other routes that involve injection.

### Nucleic Acids, Expression Vectors, Host Cells

The present disclosure provides a composition of nucleic acids comprising one or more nucleotide sequences encoding an ICBP. In some cases, where an ICBP is a heterodimeric polypeptide, the individual polypeptide chains of the ICBP are encoded in separate nucleic acids. In some cases, where an ICBP is a heterodimeric polypeptide, the individual polypeptide chains of the ICBP are encoded in a single nucleic acid. In some cases, a first nucleic acid comprises a nucleotide sequence encoding a first polypeptide of an ICBP; and a second nucleic acid comprises a nucleotide sequence encoding a second polypeptide of an ICBP.

A nucleotide sequence encoding a single-chain ICBP can be operably linked to a transcriptional control element, such as a promoter. A nucleotide sequence encoding the first polypeptide chain of a heterodimeric ICBP can be operably linked to a transcriptional control element, such as a promoter. A nucleotide sequence encoding the second polypeptide chain of a heterodimeric ICBP can be operably linked to a transcriptional control element, such as a promoter. A nucleotide sequence encoding the first and the second polypeptide chains of a heterodimeric ICBP can be operably linked to a transcriptional control element, such as a promoter. Suitable promoters include those that are functional in a eukaryotic cell. Suitable promoters include constitutive promoters and inducible promoters.

As noted above, in some cases, the individual polypeptide chains of an ICBP are encoded in separate nucleic acids. In some cases, nucleotide sequences encoding the separate polypeptide chains of an ICBP are operably linked to transcriptional control elements, e.g., promoters, such as promoters that are functional in a eukaryotic cell, where the promoter can be a constitutive promoter or an inducible promoter. In some cases, the transcriptional control element is a promoter that is functional in a eukaryotic cell. In some cases, the nucleic acids are present in separate expression vectors.

### ICBP

The present disclosure provides a nucleic acid comprising nucleotide sequences encoding the separate polypeptide chains (e.g., a first polypeptide and a second polypeptide) of an ICBP. In some cases, the nucleotide sequences encoding the first polypeptide and the second polypeptide of an ICBP includes a proteolytically cleavable linker interposed between the nucleotide sequence encoding the first polypeptide and the nucleotide sequence encoding the second polypeptide. In some cases, the nucleotide sequences encoding the first polypeptide and the second polypeptide of an ICBP includes an internal ribosome entry site (IRES) interposed between the nucleotide sequence encoding the first polypeptide and the nucleotide sequence encoding the second polypeptide. In some cases, the nucleotide sequences encoding the first polypeptide and the second polypeptide of an ICBP includes a ribosome skipping signal (or cis-acting hydrolase element, CHYSEL) interposed between the nucleotide sequence encoding the first polypeptide and the nucleotide sequence encoding the second polypeptide.

### Recombinant Expression Vectors

The present disclosure provides recombinant expression vectors comprising one or more nucleic acids as described above.

Numerous suitable expression vectors are known to those of skill in the art, and many are commercially available. See, e.g., published PCT Application WO 2020/243315 (Cue Biopharma, Inc.).

### Compositions Comprising a Nucleic Acid or a Recombinant Expression Vector

The present disclosure provides compositions, e.g., pharmaceutical compositions, comprising one or more nucleic acids or one or more recombinant expression vectors, where the nucleic acid(s) or recombinant expression vector(s) comprise a nucleotide sequence(s) encoding an ICBP of the present disclosure. A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

### Genetically Modified Host Cells

The present disclosure provides a genetically modified host cell, where the host cell is genetically modified with a nucleic acid(s) or a recombinant expression vector(s) comprising nucleotide sequences encoding an ICBP of the present disclosure.

Suitable host cells include eukaryotic cells, such as yeast cells, insect cells, and mammalian cells. In some cases, the host cell is a cell of a mammalian cell line. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), Chinese hamster ovary (CHO) cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), human embryonic kidney (HEK) cells (ATCC No. CRL1573), HLHepG2 cells, and the like.

In some cases, the host cell is a mammalian cell that has been genetically modified such that it does not synthesize endogenous β2M.

In some cases, the host cell is a mammalian cell that has been genetically modified such that it does not synthesize endogenous HLA-G heavy chain. In some cases, the host cell is a mammalian cell that has been genetically modified such that it does not synthesize endogenous β2M and such that it does not synthesize endogenous HLA-G heavy chain.

### Methods of Producing an ICBP

The present disclosure provides methods of producing an ICBP. The methods generally involve culturing, in a culture medium, a host cell that is genetically modified with one or more nucleic acids (e.g., one or more recombinant expression vectors) comprising nucleotide sequences encoding the ICBP, where the culturing is carried out under conditions that permit synthesis of the ICBP. In some case, the method comprises isolating the ICBP from the genetically modified host cell and/or the culture medium. A host cell that is genetically modified with one or more nucleic acids (e.g., one or more recombinant expression vectors) comprising nucleotide sequences encoding the ICBP is also referred to as an “expression host.” As noted above, in some cases, the individual polypeptide chains of an ICBP are encoded in separate nucleic acids (e.g., separate recombinant expression vectors). In some cases, all polypeptide chains of an ICBP are encoded in a single nucleic acid (e.g., a single recombinant expression vector).

Isolation of the ICBP from the expression host cell (e.g., from a lysate of the expression host cell) and/or the culture medium in which the host cell is cultured, can be carried out using standard methods of protein purification.

For example, a lysate may be prepared of the expression host and the lysate purified using high performance liquid chromatography (HPLC), exclusion chromatography, gel electrophoresis, affinity chromatography, or other purification technique. Alternatively, where the ICBP is secreted from the expression host cell into the culture medium, the ICBP can be purified from the culture medium using HPLC, exclusion chromatography, gel electrophoresis, affinity chromatography, or other purification technique. In some cases, the compositions which are used will comprise at least 80% by weight of the desired product (ICBP), at least about 85% by weight, at least about 95% by weight, or at least about 99.5% by weight, in relation to contaminants related to the method of preparation of the product and its purification. The percentages can be based upon total protein.

In some cases, e.g., where the ICBP comprises an affinity tag, the ICBP can be purified using an immobilized binding partner of the affinity tag.

### Methods

The present disclosure provides methods of inducing immunological tolerance in an individual, methods of reducing the activity of inflammatory immune cells in an individual, and methods of treating an autoimmune disease in an individual. The methods generally involve administering to an individual in need thereof an effective amount of an ICBP of the present disclosure.

In some cases, an “effective amount” of an ICBP is an amount that, when administered to an individual in need thereof in one or more doses, reduces the production of inflammatory cytokines (also referred to as “proinflammatory cytokines”) by immune cells in the individual. Inflammatory cytokines may be produced by T helper (Tₕ) cells and/or activated macrophages. Inflammatory cytokines include, e.g., interleukins such as IL-10, IL-6, IL-8, IL-11, IL-18; interferons such as IFN-α and IFN-β; and tumor necrosis factors such as tumor necrosis factor-alpha (TNF-α) and TNF-β. In some cases, an effective amount of an ICBP is an amount that, when administered to an individual in need thereof in one or more doses, reduces the level of inflammatory cytokines in the individual by at least 10%, at least 15%, at least 20%, at least 25%, at least 30%, at least 40%, at least 50%, or more than 50%, compared to the level of inflammatory cytokines in the individual before treatment with the ICBP.

In some cases, an effective amount of an ICBP is an amount that, when administered to an individual in need thereof in one or more doses, increases the number of regulatory T cells (Tregs) in the individual. Tregs are CD4⁺, FOXP3⁺, and CD25⁺. Tregs can suppress autoreactive T cells. In some cases, an effective amount of an ICBP is an amount that, when administered to an individual in need thereof in one or more doses, increases the number of Tregs in the individual by at least 10%, at least 15%, at least 20%, at least 25%, at least 30%, at least 40%, at least 50%, at least 60%, at least 70%, at least 80%, at least 90%, at least 2-fold, at least 2.5-fold, or more than 2.5-fold, compared to the number of Tregs in the individual before treatment with the ICBP.

In some cases, an effective amount of an ICBP is an amount that, when administered to an individual in need thereof in one or more doses, increases the number of tolerogenic dendritic cells (DCs) in the individual. In some cases, an effective amount of an ICBP is an amount that, when administered to an individual in need thereof in one or more doses, increases the number of tolerogenic DCs in the individual by at least 10%, at least 15%, at least 20%, at least 25%, at least 30%, at least 40%, at least 50%, at least 60%, at least 70%, at least 80%, at least 90%, at least 2-fold, at least 2.5-fold, or more than 2.5-fold, compared to the number of tolerogenic DCs in the individual before treatment with the ICBP. Tolerogenic DCs can be identified by known characteristics, e.g., low expression of MHC class II polypeptides compared to mature DCs; low expression of CD80/CD86 and CD40 compared to mature DCs; increased production of anti-inflammatory molecules such as IL-10 and TGF-β; and decreased production of IL-12p70 and other proinflammatory cytokines.

### Treatment Methods

In some cases, a method of the present disclosure induces immunological tolerance in an individual, and thereby treats an autoimmune disease. Thus, the present disclosure provides methods of treating an autoimmune disease in an individual, the method comprising administering to the individual an effective amount of an ICBP (or protein comprising two ICBPs).

In some cases, an “effective amount” of an ICBP is an amount that, when administered in one or more doses to an individual in need thereof, ameliorates one or more symptoms associated with the autoimmune disease in the individual. In some instances, the ICBP reduces the number of CD4+ self-reactive T cells (i.e., the number of CD4⁺ T cells reactive with an autoimmune disease-associated antigen), which in turn leads to a reduction in CD8⁺ self-reactive T cells. In some instances, the ICBP increases the number of CD4+ Tregs, which in turn reduces the number of CD4⁺ self-reactive T cells and/or CD8⁺ T self-reactive T cells.

Autoimmune diseases that potentially can be treated with a method of the present disclosure include type 1 diabetes (T1D), Addison disease (autoimmune adrenalitis, Morbus Addison), alopecia areata, Addison's anemia (Morbus Biermer), autoimmune hemolytic anemia (AIHA), autoimmune hemolytic anemia (AIHA) of the cold type (cold hemagglutinin disease, cold autoimmune hemolytic anemia (AIHA) (cold agglutinin disease), (CHAD)), autoimmune hemolytic anemia (AIHA) of the warm type (warm AIHA, warm autoimmune hemolytic anemia (AIHA)), autoimmune hemolytic Donath-Landsteiner anemia (paroxysmal cold hemoglobinuria), antiphospholipid syndrome (APS), atherosclerosis, autoimmune arthritis, arteriitis temporalis, Takayasu arteriitis (Takayasu's disease, aortic arch disease), temporal arteriitis/giant cell arteriitis, autoimmune chronic gastritis, autoimmune infertility, autoimmune inner ear disease (AIED), Basedow's disease (Morbus Basedow), Bechterew's disease (Morbus Bechterew, ankylosing spondylitis, spondylitis ankylosans), Behcet's syndrome (Morbus Behcet), bowel disease including autoimmune inflammatory bowel disease (including colitis ulcerosa (Morbus Crohn, Crohn's disease), autoimmune cardiomyopathy, idiopathic dilated cardiomyopathy (DCM), Celiac disease, chronic fatigue immune dysfunction syndrome (CFIDS), chronic inflammatory demyelinating polyneuropathy (CIDP), chronic polyarthritis, Churg-Strauss syndrome, cicatricial pemphigoid, Cogan syndrome, CREST syndrome (syndrom with Calcinosis cutis, Raynaud phenomenon, motility disorders of the esophagus, sklerodaktylia and teleangiectasia), Crohn's disease (Morbus Crohn, colitis ulcerosa), dermatitis herpetiformis during, dermatologic autoimmune diseases, dermatomyositis, essential mixed cryoglobulinemia, essential mixed cryoglobulinemia, fibromyalgia, fibromyositis, Goodpasture syndrome (anti-GBM mediated glomerulonephritis), graft versus host disease, Guillain-Barre syndrome (GBM, Polyradikuloneuritis), hematologic autoimmune diseases, Hashimoto thyroiditis, hemophilia, acquired hemophilia, autoimmune hepatitis, idiopathic pulmonary fibrosis (IPF), idiopathic thrombocytopenic purpura, Immuno-thrombocytopenic purpura (Morbus Werlhof, ITP), IgA nephropathy, autoimmune infertility, juvenile rheumatoid arthritis (Morbus Still, Still syndrome), Lambert-Eaton syndrome, systemic lupus erythematosus (SLE), lupus erythematosus (discoid form), Lyme arthritis (Lyme disease, borrelia arthritis), Meniere's disease (Morbus Meniere); mixed connective tissue disease (MCTD), multiple sclerosis (MS, encephalomyelitis disseminate, Charcot's disease), myasthenia gravis (myasthenia, MG), myositis, polymyositis, neural autoimmune diseases, pemphigus vulgaris, bullous pemphigoid, polyglandular (autoimmune) syndrome (PGA syndrome, Schmidt's syndrome), polymyalgia rheumatica, primary agammaglobulinemia, primary autoimmune cholangitis, progressive systemic sclerosis (PSS), rheumatoid arthritis (RA, chronic polyarthritis, rheumatic disease of the joints, rheumatic fever), sarcoidosis (Morbus Boeck, Besnier-Boeck-Schaumann disease), stiff-man syndrome, Sclerodermia, Scleroderma, Sjögren's syndrome, autoimmune uveiitis, and Wegner's disease (Morbus Wegner, Wegner's granulomatosis).

In some cases, a method of the present disclosure induces immunological tolerance in an individual, and thereby reduces transplant rejection and/or one or more symptoms associated with transplant rejection. Thus, the present disclosure provides a method of inducing immunological tolerance in a transplant recipient toward a transplanted cell, tissue, or organ. Transplanted organs include, e.g., skin, kidney, lung, liver, pancreas, and heart. Transplanted tissues include, e.g., small intestine, blood vessels, bone, and the like. Transplanted cells include, e.g., pancreatic islet cells, heterologous bone marrow cells, hematopoietic stem cells, and the like.

In some cases, a method of the present disclosure induces immunological tolerance in an individual, and thereby reduces one or more symptoms associated with graft versus host disease (GVH).

In some cases, a method of the present disclosure induces immunological tolerance in an individual, and thereby reduces one or more symptoms associated with metabolic diseases and disorders.

Metabolism is the chemical process that the body uses to transform food into the fuel that keeps the body alive. Nutrition (food) consists of proteins, carbohydrates, and fats. These substances are broken down by enzymes in the digestive system, and then carried to the cells where they can be used as fuel. The body either uses these substances immediately, or stores them in the liver, body fat, and muscle tissues for later use. Metabolic disorders, which can be either inherited or acquired, are disorders that interfere with the body's metabolism, and can negatively alter the body's processing and distribution of macronutrients such as proteins, fats, and carbohydrates. Metabolic disorders can happen when abnormal chemical reactions in the body alter the normal metabolic process.

There are hundreds of inherited metabolic disorders that are caused by genetic defects. Examples include familial hypercholesterolemia, Gaucher disease, Hunter syndrome, Krabbe disease, maple syrup urine disease, metachromatic leukodystrophy, cystic fibrosis, mitochondrial encephalopathy, lactic acidosis, stroke-like episodes (MELAS), Niemann-Pick, phenylketonuria (PKU), porphyria, sickle cell anemia, Tay-Sachs disease and Wilson's disease.

Acquired metabolic disorders, which are metabolic disorders that are acquired during a person's lifetime, can result from a variety of factors, including obesity. Such disorders include, e.g.: type 2 diabetes (T2D) and pre-T2D that can result from insulin resistance and/or deficient insulin secretion, and non-alcoholic fatty liver disease (NAFLD) including non-alcoholic steatohepatitis (NASH), which is a severe form of NAFLD that is closely related to obesity, pre-T2D and T2D. The ICBP can induce immunological tolerance in an individual, and thereby reduce one or more symptoms associated metabolic diseases and disorders such as type 2 diabetes (T2D) that can result from insulin resistance and/or deficient insulin secretion, and non-alcoholic fatty liver disease (NAFLD) including non-alcoholic steatohepatitis (NASH). The ICBP also can induce immunological tolerance in an individual having one or more metabolic disorders or metabolic dysregulation associated with obesity.

### Dosages

A suitable dosage of an ICBP (or a protein comprising two ICBPs, e.g., a dimerized ICBP) can be determined by an attending physician or other qualified medical personnel, based on various clinical factors. As is well known in the medical arts, dosages for any one patient depend upon many factors, including the patient's size, body surface area, age, the particular polypeptide or nucleic acid to be administered, sex of the patient, time, and route of administration, general health, and other drugs being administered concurrently. An ICBP or a dimerized ICBP may be administered in amounts between 0.1 mg/kg body weight and 20 mg/kg body weight per dose, e.g. between 0.1 mg/kg body weight to 10 mg/kg body weight, e.g. between 0.5 mg/kg body weight to 5 mg/kg body weight, between 1 mg/kg body weight to 5 mg/kg body weight; between 5 mg/kg body weight to 10 mg/kg body weight; between 10 mg/kg body weight to 15 mg/kg body weight; between 15 mg/kg body weight to 20 mg/kg body weight, however, doses above this exemplary range are envisioned, especially considering the aforementioned factors. If the regimen is a continuous infusion, it can also be in the range of 1 μg to 10 mg per kilogram of body weight per minute. An ICBP or dimerized ICBP can be administered in an amount of from about 1 mg/kg body weight to 50 mg/kg body weight, e.g., from about 1 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, from about 20 mg/kg body weight to about 25 mg/kg body weight, from about 25 mg/kg body weight to about 30 mg/kg body weight, from about 30 mg/kg body weight to about 35 mg/kg body weight, from about 35 mg/kg body weight to about 40 mg/kg body weight, or from about 40 mg/kg body weight to about 50 mg/kg body weight. Exemplary amounts of an ICBP or a dimerized ICBP include from 1 mg/kg body weight to 5 mg/kg body weight, from 5 mg/kg body weight to 10 mg/kg body weight, from about 1 mg/kg body weight to about 5 mg/kg body weight, and from about 5 mg/kg body weight to about 10 mg/kg body weight.

Persons of ordinary skill in the art can readily estimate repetition rates for dosing, e.g., based on the clinical condition of the patient, the patient's response to administration of the ICBP, and the measured residence times and concentrations of the administered agent in bodily fluids or tissues following administration. Following successful treatment, it may be desirable to have the patient undergo maintenance therapy to prevent the recurrence of the disease state, wherein an ICBP or dimerized ICBP is administered in maintenance doses, ranging from about 1 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, or amounts exceeding 20 mg/kg of body weight.

Those of skill will readily appreciate that dose levels can vary as a function of the specific ICBP or dimerized ICBP, the severity of the symptoms and the susceptibility of the subject to side effects. Preferred dosages for a given compound are readily determinable by those of skill in the art by a variety of means known to such persons.

In some cases, multiple doses of an ICBP or dimerized ICBP are administered over a course of treatment. The frequency of administration of an ICBP or dimerized ICBP can vary depending on any of a variety of factors, including those discussed above. For example, in some cases, an ICBP or a dimerized ICBP is administered once per month, twice per month, three times per month, every other week (qow), once per week (qw), once every two weeks, once every three weeks, once every four weeks, once every two months, once every three months, or less frequently than once every three months, or more frequently than once per week, e.g., twice per week (biw), three times per week (tiw), four times per week, five times per week, six times per week, every other day (qod), daily (qd), twice a day (qid), or three times a day (tid). Where the ICBP or dimerized ICBP is administered intravenously, administration once every week, once every two weeks, once every three weeks or once every four weeks or once every month may be commonly employed at the beginning of treatment.

The duration of administration of an ICBP or dimerized ICBP, e.g., the period of time over which an ICBP or dimerized ICBP is administered, can vary, depending on any of a variety of factors, e.g., patient clinical condition and response to the ICBP, etc. For example, an ICBP or a dimerized ICBP can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more, including periodic administration over the life of the patient.

### Routes of Administration

An ICBP or dimerized ICBP is administered to an individual using any available method and route suitable for drug delivery, including in vivo and in vitro methods, as well as systemic and localized routes of administration. An ICBP or dimerized ICBP of this disclosure typically will be delivered via intravenous administration, but other conventional and pharmaceutically acceptable routes of administration may be used, including intratumoral, peritumoral, intramuscular, intralymphatic, intratracheal, intracranial, subcutaneous, intradermal, topical application, intraarterial, rectal, nasal, oral, and other enteral and parenteral routes of administration.

In some cases, an ICBP or dimerized ICBP is administered intravenously. In some cases, an ICBP or dimerized ICBP is administered intramuscularly. In some cases, an ICBP or dimerized ICBP is administered subcutaneously.

### Subjects Suitable for Treatment

Subjects suitable for treatment with a method of the present disclosure include individuals who have an autoimmune disease, including individuals who have been diagnosed as having a condition that can benefit from increased immune tolerance, e.g., an autoimmune disorder, a metabolic disorder, a transplant, or GVHD, including individuals who have been treated for such conditions but have failed to respond to the treatment, and individuals who have symptoms indicating the imminent onset of one of such conditions.

For example, subjects suitable for treatment with a method of the present disclosure include individuals who have T1D, including individuals who have been diagnosed as having T1D or T2D, and individuals who have been treated for TID or T2D but who failed to respond to the treatment. Suitable subjects also may include individuals who have been diagnosed as being likely to develop T1D or T2D or who have symptoms indicating the imminent onset of T1D or T2D. Subjects suitable for treatment with a method of the present disclosure include individuals who have T1D or T2D, including individuals who have been diagnosed as having Celiac disease, and individuals who have been treated for Celiac disease but who failed to respond to the treatment. Suitable subjects also may include individuals who have been diagnosed as being likely to develop Celiac disease or who have symptoms indicating the imminent onset of Celiac disease.

Other subjects suitable for treatment with a method of the present disclosure include transplant recipients, and individuals who are about to become transplant recipients (e.g., individuals who are scheduled to receive a transplant within a month or less, or within a week or less).

Other subjects suitable for treatment with a method of the present disclosure include bone marrow transplant recipients, and individuals who are about to become bone marrow transplant recipients (e.g., individuals who are scheduled to receive a bone marrow transplant within a month or less, or within a week or less).

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. An immune cell binding polypeptide (ICBP) comprising: a) a binding moiety comprising an HLA-G molecule and a stabilizing peptide; b) optionally, at least one immunoinhibitory polypeptide; c) a scaffold polypeptide, and d) optionally, one or more independently selected linkers linking two or more of the components of the ICBP, wherein the HLA-G molecule comprises an HLA-G heavy chain polypeptide and a β2M polypeptide, and wherein the binding moiety can bind to ILT2 and/or ILT4 polypeptides on an immune cell, and wherein the ICBP can suppress the activity of the immune cell.

Aspect 2. The ICBP of aspect 1, wherein the scaffold polypeptide is an immunoglobulin (Ig) Fc polypeptide.

Aspect 3. The ICBP of aspect 2, wherein the Ig Fc polypeptide is a human IgG1 Fc polypeptide that substantially does not induce cell lysis, optionally wherein the IgG1 Fc polypeptide comprises an amino acid sequence having at least 90% amino acid sequence identity to the IgG1 Fc amino acid sequence depicted in FIG. 2A.

Aspect 4. The ICBP of aspect 3, wherein the IgG1 Fc polypeptide comprises one or more amino acid substitutions selected from N77A, L14A, L15A, L14F, L15E, and P101S, based on the numbering of the IgG1 Fc amino acid sequence depicted in FIG. 2A.

Aspect 5. The ICBP of aspect 4, wherein the IgG1 Fc polypeptide comprises amino acid substitutions L14A and L15A based on the numbering of the Ig Fe amino acid sequence depicted in FIG. 2A.

Aspect 6. The ICBP of any one of aspects 1-5, wherein the β2M polypeptide and the HLA-G heavy chain polypeptide are joined by a disulfide bond that joins a Cys residue in the Q2M polypeptide and a Cys residue in the HLA-G heavy chain polypeptide.

Aspect 7. The ICBP of aspect 6, wherein a Cys at amino acid residue 12 of the β2M polypeptide is disulfide bonded to a Cys at amino acid residue 236 of the HLA-G heavy chain polypeptide.

Aspect 8. The ICBP of any one of aspects 1-7, wherein the β2-microglobulin polypeptide is joined to the stabilizing peptide by a first linker comprising a Cys, and wherein a disulfide bond links a Cys present in the first linker with a Cys present in the HLA-G heavy chain polypeptide.

Aspect 9. The ICBP of aspect 8, wherein the first linker comprises the sequence CGGGS(GGGGS)n (SEQ ID NO:146) or GCGGS(GGGGS)n (SEQ ID NO:144), where n is an integer from 1-10, e.g., 2 or 3, and a disulfide bond links the Cys in the linker with a Cys substituted for Tyr84 of the HLA-G heavy chain polypeptide.

Aspect 10. The ICBP of any one of aspects 1-9, wherein the HLA-G heavy chain polypeptide comprises an amino acid sequence having at least 90% amino acid sequence identity to the HLA-G heavy chain polypeptide depicted in any one of FIG. 4A-4F, and optionally wherein the HLA-G heavy chain polypeptide comprises a C42S amino acid substitution.

Aspect 11. The ICBP of any one of aspects 1-10, wherein the ICBP comprises at least one immunoinhibitory polypeptide that can bind to and mediate inhibition of an immune cell, optionally wherein the one or more immunoinhibitory polypeptides are selected from a PD-L1 polypeptide, a FasL polypeptide, an IL-10 polypeptide, an IL-35 polypeptide, a TIGIT ligand, a Lag3 ligand, and combinations thereof.

Aspect 12. The ICBP of any one of aspects 1-11, wherein at least one of the one or more immunoinhibitory polypeptides binds to it cognate co-receptor and exhibits reduced affinity to its cognate co-receptor compared to the affinity of a corresponding wild-type immunoinhibitory polypeptide for the cognate co-receptor polypeptide.

Aspect 13. The ICBP of any one of aspects 1-12, wherein at least one of the one or more immunoinhibitory polypeptides is a PD-L1 polypeptide, optionally wherein the immunoinhibitory polypeptide comprises a PD-L1 polypeptide extracellular domain.

Aspect 14. The ICBP of aspect 13, wherein the PD-L1 polypeptide comprises an amino acid sequence having at least 95% amino acid sequence identity to the PD-L1 amino acid sequence depicted in FIG. 11A or FIG. 11C, and has a length of about 220 amino acids.

Aspect 15. The ICBP of any one of aspects 1-14, wherein the ICBP is a single polypeptide chain.

Aspect 16. The ICBP of any one of aspects 1-14, wherein the ICBP is a heterodimeric polypeptide.

Aspect 17. The ICBP of any one of aspects 1-16, wherein the β2-microglobulin polypeptide is joined to the stabilizing peptide by a first linker that comprises the sequence CGGGS(GGGGS)n (SEQ ID NO:146) or GCGGS(GGGGS)n (SEQ ID NO:144), where n is an integer from 1-10, e.g., 2 or 3, wherein the HLA-G polypeptide comprises a Cys at residue 84 and a Cys at residue 236, wherein the β2M polypeptide comprises a Cys at residue 12, wherein the Cys at amino acid residue 12 of the β2M polypeptide is disulfide bonded to a Cys at amino acid residue 236 of the HLA-G polypeptide, wherein a disulfide bond links the Cys in the linker with a Cys substituted for Tyr84 of the HLA-G polypeptide, wherein the HLA-G polypeptide comprises a serine at amino acid residue 42, wherein the at least one immunoinhibitory polypeptide is a PD-L1 polypeptide, and wherein the Ig Fc polypeptide is an IgG1 Fc polypeptide that comprises L14A and L15A substitutions.

Aspect 18. An ICBP according to any one of Aspects 1-10, wherein the ICBP comprises: a) a binding moiety comprising an HLA-G molecule and a stabilizing peptide; and b) a scaffold polypeptide, but does not comprise an immunoinhibitory polypeptide, wherein the HLA-G molecule comprises an HLA-G heavy chain polypeptide and a β2M polypeptide, and wherein the binding moiety can bind to ILT2 and/or ILT4 polypeptides on an immune cell.

Aspect 19. A dimer comprising two ICBPs according to any one of aspects 1-18, optionally wherein the dimer is a homodimer of two ICBPs that have the same amino acid sequence and wherein the two polypeptides of the homodimer are joined to each other by one or more disulfide bonds that join the scaffold polypeptide of one polypeptide to the scaffold polypeptide of the other polypeptide.

Aspect 20. A pharmaceutical composition comprising an ICBP of any one of aspects 1-18.

Aspect 21. A pharmaceutical composition comprising a dimer of aspect 19.

Aspect 22. A method of producing an ICBP according to any one of aspects 1-18, or a dimer of aspect 19, the method comprising culturing in vitro a host cell that is genetically modified with one or more nucleic acids comprising nucleotide sequences encoding the one or more polypeptides of the ICBP, wherein the genetically modified host cell produces the one or more polypeptides of the ICBP.

Aspect 23. A method of inducing immune tolerance in an individual, the method comprising administering to the individual an ICBP of any one of aspects 1-18, a dimer of aspect 19, or a pharmaceutical composition of aspect 20 or aspect 21.

Aspect 24. A method of aspect 23, wherein the individual has an autoimmune disease.

Aspect 25. A method of aspect 23, wherein the individual is a transplant recipient.

Aspect 26. A method of aspect 23, wherein the individual has graft-versus host (GVH) disease.

Aspect 27. A method of aspect 23, wherein the individual has a metabolic disorder such as T2D and/or non-alcoholic fatty liver disease (NAFLD) including non-alcoholic steatohepatitis (NASH).

Aspect 28. A method of any one of aspects 23-27, wherein said administering increases the number of regulatory T cells in the individual.

Aspect 29. A method of any one of aspects 23-27, wherein said administering increases the number of tolerogenic dendritic cells in the individual.

Aspect 30. A method of any one of aspects 23-27, wherein said administering reduces activity of inflammatory immune cells in the individual.

Aspect 31. A method of any one of aspects 23-27, wherein said administering reduces the level of proinflammatory cytokines in the individual.

### EXAMPLES

The following examples are put forth so as to provide those of ordinary skill in the art with a complete disclosure and description of how to make and use the present disclosure, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

Two constructs were generated, designated “4601+4602” and “4607+4602.” The polypeptide chain 4601 comprises an HLA-G polypeptide with C42S, Y84C, and A236C substitutions; an AAAGG (SEQ ID NO:28) linker; and an IgG1 Fc polypeptide with L14A and L15A substitutions. The polypeptide chain 4607 comprises a PD-L1 polypeptide; a (GGGGS)4 (SEQ ID NO:27) linker; an HLA-G polypeptide with C42S, Y84C, and A236C substitutions; an AAAGG (SEQ ID NO:28) linker; and an IgG1 Fc polypeptide with L14A and L15A substitutions. The polypeptide chain 4602 comprises a histone 2A peptide (RIIPRHLQL; SEQ ID NO:24); a linker having the amino acid sequence GCGGSGGGGSGGGGS (SEQ ID NO:25); and a β2M polypeptide with an R12C substitution. The amino acid sequences of the polypeptide chains of 4601+4602 are provided in FIG. 6A. The amino acid sequences of the polypeptide chains of 4607+4602 are provided in FIG. 6B.

As discussed above, each of these heterodimeric ICBPs will form interchain disulfide bonds (i) between the Cys at residue 84 of the HLA-G heavy chain and the Cys in the linker joining the stabilizing peptide RIIPRHLQL (SEQ ID NO:24) to the β2M polypeptide, (ii) between the Cys at residue 236 of the HLA-G heavy chain and the Cys residue at amino acid residue 12 of the β2M. Upon expression, ICBP construct 4601+4602 will spontaneously dimerize to form a homodimer in which disulfide bonds join the IgG1 Fc polypeptide of one ICBP to the IgG1 Fc of the other ICBP. Likewise, upon expression, ICBP construct 4607+4602 also will spontaneously dimerize to form a homodimer in which disulfide bonds join the IgG1 Fc polypeptide of one ICBP to the IgG1 Fc of the other ICBP.

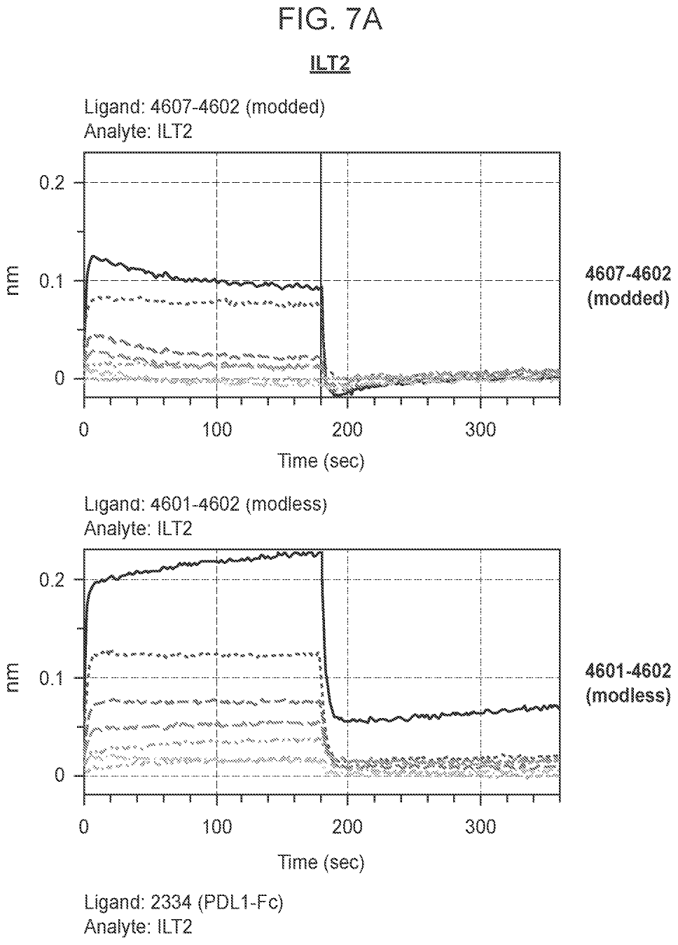

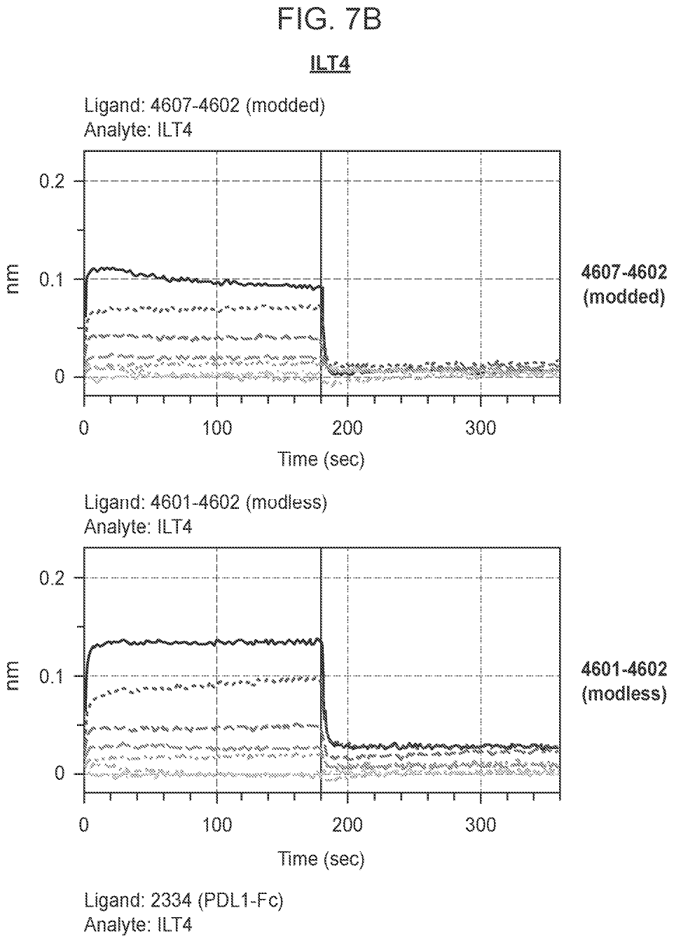

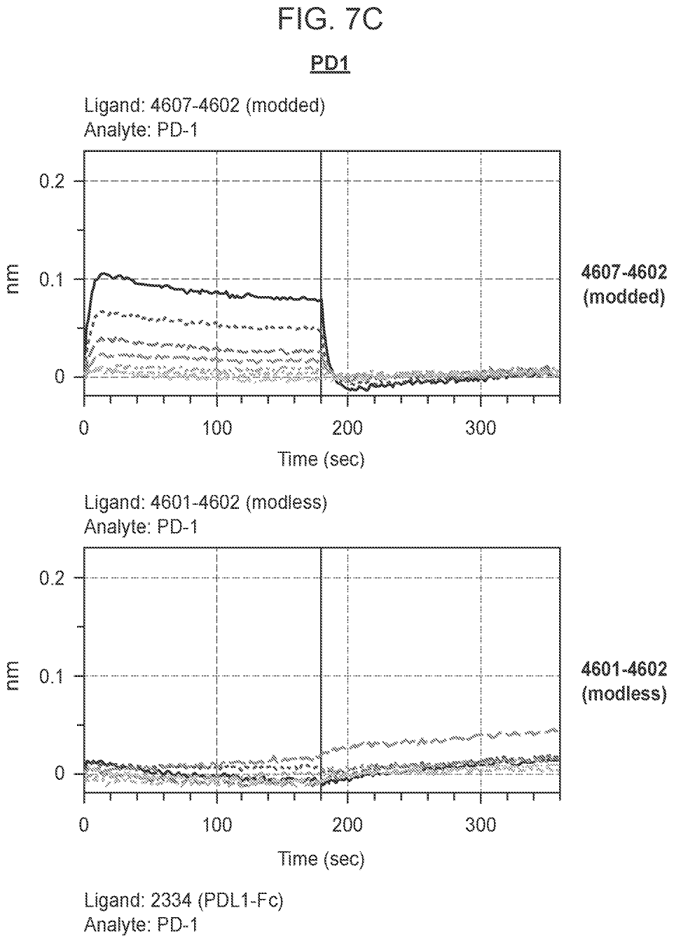

Binding of the 4601+4602 construct and the 4607+4602 construct to immobilized ILT2, ILT4, and PD1 was tested. As shown in FIG. 7, both 4601+4602 and 4607+4602 bind to both ILT2 and ILT4, but only 4607+4602 (and not 4601+4602) binds to PD1.

While the present disclosure has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present disclosure. All such modifications are intended to be within the scope of the claims appended hereto.

What is claimed is:

1. An immune cell binding polypeptide (ICBP) comprising:
a) a binding moiety comprising an HLA-G molecule and a stabilizing peptide;
b) at least one immunoinhibitory polypeptide; and
c) a scaffold polypeptide
wherein the HLA-G molecule comprises an HLA-G heavy chain polypeptide and a β2M polypeptide, and
wherein the binding moiety can bind to ILT2 and/or ILT4 polypeptides on an immune cell, and
wherein the ICBP can suppress the activity of the immune cell.

2. The ICBP of claim 1, wherein the scaffold polypeptide is an immunoglobulin (Ig) Fc polypeptide.

3. The ICBP of claim 2, wherein the Ig Fc polypeptide is a human IgG1 Fc polypeptide that substantially does not induce cell lysis, optionally wherein the IgG1 Fc polypeptide comprises an amino acid sequence having at least 90% amino acid sequence identity to the IgG1 Fc amino acid sequence depicted in FIG. 2A.

4. The ICBP of claim 3, wherein the IgG1 Fc polypeptide comprises one or more amino acid substitutions selected from N77A, L14A, L15A, L14F, L15E, and P101S, based on the numbering of the IgG1 Fc amino acid sequence depicted in FIG. 2A.

5. The ICBP of claim 4, wherein the IgG1 Fc polypeptide comprises amino acid substitutions L14A and L15A based on the numbering of the Ig Fc amino acid sequence depicted in FIG. 2A.

6. The ICBP of any one of claims 1-5, wherein the β2M polypeptide and the HLA-G heavy chain polypeptide are joined by a disulfide bond that joins a Cys residue in the β2M polypeptide and a Cys residue in the HLA-G heavy chain polypeptide.

7. The ICBP of claim 6, wherein a Cys at amino acid residue 12 of the β2M polypeptide is disulfide bonded to a Cys at amino acid residue 236 of the HLA-G heavy chain polypeptide.

8. The ICBP of any one of claims 1-7, wherein the β2-microglobulin polypeptide is joined to the stabilizing peptide by a first linker comprising a Cys, and wherein a disulfide bond links a Cys present in the first linker with a Cys present in the HLA-G heavy chain polypeptide.

9. The ICBP of claim 8, wherein the first linker comprises the sequence CGGGS(GGGGS)n (SEQ ID NO:146) or GCGGS(GGGGS)n (SEQ ID NO:144), where n is an integer from 1-10, e.g., 2 or 3, and a disulfide bond links the Cys in the linker with a Cys substituted for Tyr84 of the HLA-G heavy chain polypeptide.

10. The ICBP of any one of claims 1-9, wherein the HLA-G heavy chain polypeptide comprises an amino acid sequence having at least 90% amino acid sequence identity to the HLA-G heavy chain polypeptide depicted in any one of FIG. 4A-4F, and optionally wherein the HLA-G heavy chain polypeptide comprises a C42S amino acid substitution.

11. The ICBP of any one of claims 1-10, wherein the one or more immunoinhibitory polypeptides can bind to and mediate inhibition of an immune cell, optionally wherein the one or more immunoinhibitory polypeptides are selected from a PD-L1 polypeptide, a FasL polypeptide, an IL-10 polypeptide, an IL-35 polypeptide, a TIGIT ligand, a Lag3 ligand, and combinations thereof.

12. The ICBP of any one of claims 1-11, wherein at least one of the one or more immunoinhibitory polypeptides binds to its cognate co-receptor and exhibits reduced affinity to its cognate co-receptor compared to the affinity of a corresponding wild-type immunoinhibitory polypeptide for the cognate co-receptor polypeptide.

13. The ICBP of any one of claims 1-12, wherein at least one of the one or more ICBPs is a PD-L1 polypeptide, optionally wherein the immunoinhibitory polypeptide comprises a PD-L1 polypeptide extracellular domain.

14. The ICBP of claim 13, wherein the immunoinhibitory polypeptide comprises an amino acid sequence having at least 95% amino acid sequence identity to the PD-L1 amino acid sequence depicted in FIG. 11A or FIG. 11C, and has a length of about 220 amino acids.

15. The ICBP of any one of claims 1-14, wherein the ICBP is a single polypeptide chain.

16. The ICBP of any one of claims 1-14, wherein the ICBP is a heterodimeric polypeptide.

17. The ICBP of any one of claims 1-16, wherein the β2-microglobulin polypeptide is joined to the stabilizing peptide by a first linker that comprises the sequence CGGGS(GGGGS)n (SEQ ID NO:146) or GCGGS(GGGGS)n (SEQ ID NO:144), where n is an integer from 1-10, e.g., 2 or 3,
wherein the HLA-G polypeptide comprises a Cys at residue 84 and a Cys at residue 236,
wherein the β2M polypeptide comprises a Cys at residue 12,
wherein the Cys at amino acid residue 12 of the β2M polypeptide is disulfide bonded to a Cys at amino acid residue 236 of the HLA-G polypeptide,
wherein a disulfide bond links the Cys in the linker with a Cys substituted for Tyr84 of the HLA-G polypeptide,
wherein the HLA-G polypeptide comprises a serine at amino acid residue 42,
wherein the at least one immunoinhibitory polypeptide is a PD-L1 polypeptide, and
wherein the Ig Fc polypeptide is an IgG1 Fc polypeptide that comprises L14A and L15A substitutions.

18. A homodimer comprising two ICBPs according to any one of claims 1-17, wherein the two polypeptides are joined to each other by one or more disulfide bonds that join the scaffold polypeptide of one polypeptide to the scaffold polypeptide of the other polypeptide.

19. A pharmaceutical composition comprising an ICBP of any one of claims 1-17.

20. A pharmaceutical composition comprising a homodimer of claim 18.

21. A composition comprising nucleic acids encoding an ICBP of any one of claims 15-17.

22. One or more expression vectors comprising nucleic acids encoding an ICBP of any one of claims 15-17.

23. A method of producing an ICBP according to any one of claims 1-17, or a homodimer of claim 18, the method comprising culturing in vitro a host cell that is genetically modified with one or more nucleic acids comprising nucleotide sequences encoding the ICBP, wherein the genetically modified host cell produces the polypeptide.

24. A method of inducing immune tolerance in an individual, the method comprising administering to the individual an ICBP of any one of claims 1-17, a homodimer of claim 18, or a pharmaceutical composition of claim 19 or claim 20.

25. A method of claim 22, wherein the individual has an autoimmune disease.

26. A method of claim 22, wherein the individual is a transplant recipient.

27. A method of any one of claims 22-24, wherein said administering increases the number of regulatory T cells in the individual.

28. A method of any one of claims 22-24, wherein said administering increases the number of tolerogenic dendritic cells in the individual.

29. A method of any one of claims 22-24, wherein said administering reduces activity of inflammatory immune cells in the individual.

30. A method of any one of claims 22-24, wherein said administering reduces the level of proinflammatory cytokines in the individual.


SEQUENCE LISTING (153 sequences)

SEQ ID NO: 1 - protein, 99 aa, Homo sapiens [sequence omitted]

SEQ ID NO: 2 - protein, 99 aa, synthetic construct
IQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 3 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 227 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 325 aa, Homo sapiens
STKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 246 aa, Homo sapiens
HKPSNTKVDKRVELKTPLGDTTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 383 aa, Homo sapiens
PTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 12 - protein, 276 aa, Homo sapiens
VTSTLTIKZSDWLGESMFTCRVDHRGLTFQQNASSMCVPDQDTAIRVFAIPPSFASIFLTKSTKLTCLVTDLTTYBSVTISWTREENGAVKTHTNISESHPNATFSAVGEASICEDBDWSGERFTCTVTHTDLPSPLKQTISRPKGVALHRPBVYLLPPARZZLNLRESATITCLVTGFSPADVFVEWMQRGEPLSPQKYVTSAPMPEPQAPGRYFAHSILTVSEEEWNTGGTYTCVVAHEALPNRVTERTVDKSTGKPTLYNVSLVMSDTAGTCY

SEQ ID NO: 13 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 14 - protein, 222 aa, Homo sapiens
ADPCDSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 15 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 286 aa, synthetic construct
  REGION 1..286: note = synthetic sequence
  VARIANT 13: note = X is S or F
  VARIANT 27: note = X is Y or H
  VARIANT 31: note = X is T, S or M
  VARIANT 34: note = X is L or V
  VARIANT 54: note = X is Q or R
  VARIANT 82: note = X is P or L
  VARIANT 101: note = X is G or D
  VARIANT 105: note = X is G or V
  VARIANT 106: note = X is S or C
  VARIANT 111: note = X is L or I
  VARIANT 160: note = X is Y or H
  VARIANT 170: note = X is H or R
  VARIANT 172: note = X is Y or H
  VARIANT 179: note = X is M or T
  VARIANT 186: note = X is P or A
  VARIANT 221: note = X is R, W or Q
  VARIANT 260: note = X is T or M
  VARIANT 277: note = X is K or E
GSHSMRYFSAAVXRPGRGEPRFIAMGXVDDXQFXRFDSDSACPRMEPRAPWVEXSEGPEYWEEETRNTKAHAQTDRMNLQTXRGYYNQSEASSHTLQWMIXCDLXXDGRLXRGYEQYAYDGKDYLALNEDLRSWTAADTAAQISKRKCEAANVAEQRRAXLEGTCVEWLXRXLENGKEXLQRADPXSKTHVTHHPVFDYEATLRCWALGFYPAEIILTWQXDGEDQTQDVELVETRPAGDGTFQKWAAVVVPSGEEQRYXCHVQHEGLPEPLMLRWXQSSLPTIPI

SEQ ID NO: 17 - protein, 275 aa, Homo sapiens
GSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWK

SEQ ID NO: 18 - protein, 275 aa, synthetic construct
GSHSMRYFSAAVSRPGRGEPRFIAMGYVDDTQFVRFDSDSACPRMEPRAPWVEQEGPEYWEEETRNTKAHAQTDRMNLQTLRGCYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPCGDGTFQKWAAVVVPSGEEQRYTCHVQHEGLPEPLMLRWK

SEQ ID NO: 19 - protein, 275 aa, synthetic construct
GSHSMRYFSAAVSRPGRGEPRFIAMGYVDDTQFVRFDSDSASPRMEPRAPWVEQEGPEYWEEETRNTKAHAQTDRMNLQTLRGCYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPCGDGTFQKWAAVVVPSGEEQRYTCHVQHEGLPEPLMLRWK

SEQ ID NO: 20 - protein, 275 aa, Homo sapiens
GSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLIRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWK

SEQ ID NO: 21 - protein, 275 aa, synthetic construct
GSHSMRYFSAAVSRPGRGEPRFIAMGYVDDTQFVRFDSDSACPRMEPRAPWVEQEGPEYWEEETRNTKAHAQTDRMNLQTLRGCYNQSEASSHTLQWMIGCDLGSDGRLIRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPCGDGTFQKWAAVVVPSGEEQRYTCHVQHEGLPEPLMLRWK

SEQ ID NO: 22 - protein, 275 aa, synthetic construct
GSHSMRYFSAAVSRPGRGEPRFIAMGYVDDTQFVRFDSDSASPRMEPRAPWVEQEGPEYWEEETRNTKAHAQTDRMNLQTLRGCYNQSEASSHTLQWMIGCDLGSDGRLIRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPCGDGTFQKWAAVVVPSGEEQRYTCHVQHEGLPEPLMLRWK

SEQ ID NO: 23 - protein, 884 aa, synthetic construct
RIIPRHLQLGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFSAAVSRPGRGEPRFIAMGYVDDTQFVRFDSDSASPRMEPRAPWVEQEGPEYWEEETRNTKAHAQTDRMNLQTLRGCYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPCGDGTFQKWAAVVVPSGEEQRYTCHVQHEGLPEPLMLRWKGGGGSGGGGSGGGGSGGGGSFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 9 aa, synthetic construct
RIIPRHLQL

SEQ ID NO: 25 - protein, 15 aa, synthetic construct
GCGGSGGGGSGGGGS

SEQ ID NO: 26 - protein, 15 aa, synthetic construct
GGGGSGGGGSGGGGS

SEQ ID NO: 27 - protein, 20 aa, synthetic construct
GGGGSGGGGSGGGGSGGGGS

SEQ ID NO: 28 - protein, 5 aa, synthetic construct
AAAGG

SEQ ID NO: 29 - protein, 884 aa, synthetic construct
RIIPRHLQLGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFSAAVSRPGRGEPRFIAMGYVDDTQFVRFDSDSASPRMEPRAPWVEQEGPEYWEEETRNTKAHAQTDRMNLQTLRGCYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPCGDGTFQKWAAVVVPSGEEQRYTCHVQHEGLPEPLMLRWKGGGGSGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAAAGGFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 30 - protein, 506 aa, synthetic construct
GSHSMRYFSAAVSRPGRGEPRFIAMGYVDDTQFVRFDSDSASPRMEPRAPWVEQEGPEYWEEETRNTKAHAQTDRMNLQTLRGCYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPCGDGTFQKWAAVVVPSGEEQRYTCHVQHEGLPEPLMLRWKAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 31 - protein, 123 aa, synthetic construct
RIIPRHLQLGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 32 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 33 - protein, 746 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERGGGGSGGGGSGGGGSGGGGSGSHSMRYFSAAVSRPGRGEPRFIAMGYVDDTQFVRFDSDSASPRMEPRAPWVEQEGPEYWEEETRNTKAHAQTDRMNLQTLRGCYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPCGDGTFQKWAAVVVPSGEEQRYTCHVQHEGLPEPLMLRWKAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 34 - protein, 220 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 35 - protein, 746 aa, synthetic construct
GSHSMRYFSAAVSRPGRGEPRFIAMGYVDDTQFVRFDSDSASPRMEPRAPWVEQEGPEYWEEETRNTKAHAQTDRMNLQTLRGCYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPCGDGTFQKWAAVVVPSGEEQRYTCHVQHEGLPEPLMLRWKGGGGSGGGGSGGGGSGGGGSFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 36 - protein, 650 aa, Homo sapiens
MTPILTVLICLGLSLGPRTHVQAGHLPKPTLWAEPGSVITQGSPVTLRCQGGQETQEYRLYREKKTAPWITRIPQELVKKGQFPIPSITWEHTGRYRCYYGSDTAGRSESSDPLELVVTGAYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 37 - protein, 652 aa, Homo sapiens
MTPILTVLICLGLSLGPRTHVQAGHLPKPTLWAEPGSVITQGSPVTLRCQGGQETQEYRLYREKKTAPWITRIPQELVKKGQFPIPSITWEHTGRYRCYYGSDTAGRSESSDPLELVVTGAYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSAGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRQSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 38 - protein, 651 aa, Homo sapiens
MTPILTVLICLGLSLGPRTHVQAGHLPKPTLWAEPGSVITQGSPVTLRCQGGQETQEYRLYREKKTAPWITRIPQELVKKGQFPIPSITWEHTGRYRCYYGSDTAGRSESSDPLELVVTGAYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSAGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 39 - protein, 651 aa, Homo sapiens
MTPILTVLICLGLSLGPRTHVQAGHLPKPTLWAEPGSVITQGSPVTLRCQGGQETQEYRLYREKKTAPWITRIPQELVKKGQFPIPSITWEHTGRYRCYYGSDTAGRSESSDPLELVVTGAYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRQSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 40 - protein, 634 aa, Homo sapiens
MTPILTVLICLGLSLGPRTHVQAGHLPKPTLWAEPGSVITQGSPVTLRCQGGQETQEYRLYREKKTAPWITRIPQELVKKGQFPIPSITWEHTGRYRCYYGSDTAGRSESSDPLELVVTGAYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSAGPEDQPLTPTGSDPQSGLGRHLGVVIGILVAVILLLLLLLLLFLILRHRRQGKHWTSTQRKADFQHPAGAVGPEPTDRGLQWRSSPAADAQEENLYAAVKHTQPEDGVEMDTRSPHDEDPQAVTYAEVKHSRPRREMASPPSPLSGEFLDTKDRQAEEDRQMDTEAAASEAPQDVTYAQLHSLTLRREATEPPPSQEGPSPAVPSIYATLAIH

SEQ ID NO: 41 - protein, 456 aa, Homo sapiens
MTPILTVLICLGLSLGPRTHVQAGHLPKPTLWAEPGSVITQGSPVTLRCQGGQETQEYRLYREKKTAPWITRIPQELVKKGQFPIPSITWEHTGRYRCYYGSDTAGRSESSDPLELVVTGAYIKPTLSAQPSPVVNSGGNVTLQCDSQVAFDGFILCKEGEDEHPQCLNSQPHARGSSRAIFSVGPVSPSRRWWYRCYAYDSNSPYEWSLPSDLLELLVLGVSKKPSLSVQPGPIVAPEETLTLQCGSDAGYNRFVLYKDGERDFLQLAGAQPQAGLSQANFTLGPVSRSYGGQYRCYGAHNLSSEWSAPSDPLDILIAGQFYDRVSLSVQPGPTVASGENVTLLCQSQGWMQTFLLTKEGAADDPWRLRSTYQSQKYQAEFPMGPVTSAHAGTYRCYGSQSSKPYLLTHPSDPLELVVSGPSGGPSSPTTGPTSTSAGPEDQPLTPTGSDPQSGE

SEQ ID NO: 42 - protein, 598 aa, Homo sapiens
MTPIVTVLICLGLSLGPRTRVQTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEDEHPQCLNSQPHARGSSRAIFSVGPVSPNRRWSHRCYGYDLNSPYVWSSPSDLLELLVPGVSKKPSLSVQPGPVMAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 43 - protein, 597 aa, Homo sapiens
MTPIVTVLICLGLSLGPRTRVQTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEDEHPQCLNSQPHARGSSRAIFSVGPVSPNRRWSHRCYGYDLNSPYVWSSPSDLLELLVPGVSKKPSLSVQPGPVMAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 44 - protein, 482 aa, Homo sapiens
MTGAYPKPTLSAQPSPVVTSGGRVTLQCESQVAFGGFILCKEGEDEHPQCLNSQPHARGSSRAIFSVGPVSPNRRWSHRCYGYDLNSPYVWSSPSDLLELLVPGVSKKPSLSVQPGPVMAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

SEQ ID NO: 45 - protein, 510 aa, Homo sapiens
MTPIVTVLICLGLSLGPRTRVQTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEDEHPQCLNSQPHARGSSRAIFSVGPVSPNRRWSHRCYGYDLNSPYVWSSPSDLLELLVPGVSKKPSLSVQPGPVMAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGLGRHLGVVIGILVAVVLLLLLLLLLFLILRHRRQGKHWTSSPAQLPTPRKKTSMLP

SEQ ID NO: 46 - protein, 455 aa, Homo sapiens
MTPIVTVLICLGLSLGPRTRVQTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEDEHPQCLNSQPHARGSSRAIFSVGPVSPNRRWSHRCYGYDLNSPYVWSSPSDLLELLVPGVSKKPSLSVQPGPVMAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPAGPEDQPLTPTGSDPQSGE

SEQ ID NO: 47 - protein, 345 aa, Homo sapiens
DVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGTEHASASANGHVSYSAVSRENSSSQDPQTEGTR

SEQ ID NO: 48 - protein, 337 aa, Homo sapiens
DVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKGTEHASASANGHVSYSAVSRENSSSQDPQTEGTR

SEQ ID NO: 49 - protein, 329 aa, Homo sapiens
QDVRVQVLPEVRGQLGGTVELPCHLLPPVPGLYISLVTWQRPDAPANHQNVAAFHPKMGPSFPSPKPGSERLSFVSAKQSTGQDTEAELQDATLALHGLTVEDEGNYTCEFATFPKGSVRGMTWLRVIAKPKNQAEAQKVTFSQDPTTVALCISKEGRPPARISWLSSLDWEAKETQVSGTLAGTVTVTSRFTLVPSGRADGVTVTCKVEHESFEEPALIPVTLSVRYPPEVSISGYDDNWYLGRTDATLSCDVRSNPEPTGYDWSTTSGTFPTSAVAQGSQLVIHAVDSLFNTTFVCTVTNAVGMGRAEQVIFVRETPNTAGAGATGG

SEQ ID NO: 50 - protein, 329 aa, Homo sapiens
QDVRVQVLPEVRGQLGGTVELPCHLLPPVPGLYISLVTWQRPDAPANHQNVAAFHPKMGPSFPSPKPGSERLSFVSAKQSTGQDTEAELQDATLALHGLTVEDEGNYTCEFATFPKGSVRGMTWLRVIAKPKNQAEAQKVTFSQDPTTVALCISKEGRPPARISWLSSLDWEAKETQVSGTLAGTVTVTSRFTLVPSGRADGVTVTCKVEHESFEEPALIPVTLSVRYPPEVSISGYDDNWYLGRTDATLSCDVRSNPEPTGYDWSTTSGTFPTSAVAQGSQLVIHAVDSLFNTTFVCTVTNAVGMGRAEQVIFVRETPRASPRDVGPL

SEQ ID NO: 51 - protein, 290 aa, Homo sapiens
LEDCAQEQMRLRAQVRLLETRVKQQQVKIKQLLQENEVQFLDKGDENTVIDLGSKRQYADCSEIFNDGYKLSGFYKIKPLQSPAEFSVYCDMSDGGGWTVIQRRSDGSENFNRGWKDYENGFGNFVQKHGEYWLGNKNLHFLTTQEDYTLKIDLADFEKNSRYAQYKNFKVGDEKNFYELNIGEYSGTAGDSLAGNFHPEVQWWASHQRMKFSTWDRDHDNYEGNCAEEDQSGWWFNRCHSANLNGVYYSGPYTAKTDNGIVWYTWHGWWYSLKSVVMKIRPNDFIPNVI

SEQ ID NO: 52 - protein, 239 aa, Homo sapiens
KASTERAALLDGHDLLRTNASKQTAALGALKEEVGDCHSCCSGTQAQLQTTRAELGEAQAKLMEQESALRELRERVTQGLAEAGRGREDVRTELFRALEAVRLQNNSCEPCPTSWLSFEGSCYFFSVPKTTWAAAQDHCADASAHLVIVGGLDEQGFLTRNTRGRGYWLGLRAVRHLGKVQGYQWVDGVSLSFSHWNQGEPNDAWGRENCVMMLHTGLWNDAPCDSEKDGWICEKRHNC

SEQ ID NO: 53 - protein, 220 aa, synthetic construct
  VARIANT 8: note = where X is any amino acid other than Asp
  VARIANT 19: note = where X is any amino acid other than Thr
  VARIANT 36: note = where X is any amino acid other than Ile
  VARIANT 42: note = where X is any amino acid other than Glu
  VARIANT 48: note = where X is any amino acid other than Gln
  VARIANT 54: note = where X is any amino acid other than Glu
  VARIANT 98: note = where X is any amino acid other than Ile
  VARIANT 101: note = where X is any amino acid other than Gly
  VARIANT 102: note = where X is any amino acid other than Gly
  VARIANT 103: note = where X is any amino acid other than Ala
  VARIANT 104: note = where X is any amino acid other than Asp
  VARIANT 105: note = where X is any amino acid other than Tyr
  VARIANT 106: note = where X is any amino acid other than Lys
  VARIANT 107: note = where X is any amino acid other than Arg
FTVTVPKXLYVVEYGSNMXIECKFPVEKQLDLAALXVYWEMXDKNIIXFVHGEXDLKVQHSSYRQRARLLKDQLSLGNAALQITDVKLQDAGVYRCMXSYXXXXXXXITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 54 - protein, 219 aa, Homo sapiens
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKI

SEQ ID NO: 55 - protein, 217 aa, synthetic construct
  VARIANT 8: note = where X is any amino acid other than Asp
  VARIANT 19: note = where X is any amino acid other than Thr
  VARIANT 36: note = where X is any amino acid other than Ile
  VARIANT 42: note = where X is any amino acid other than Glu
  VARIANT 48: note = where X is any amino acid other than Gln
  VARIANT 54: note = where X is any amino acid other than Glu
  VARIANT 98: note = where X is any amino acid other than Ile
  VARIANT 99: note = where X is any amino acid other than Gly
  VARIANT 100: note = where X is any amino acid other than Gly
  VARIANT 101: note = where X is any amino acid other than Ala
  VARIANT 102: note = where X is any amino acid other than Asp
  VARIANT 103: note = where X is any amino acid other than Tyr
  VARIANT 104: note = where X is any amino acid other than Lys
  VARIANT 105: note = where X is any amino acid other than Arg
FTVTVPKXLYVVEYGSNMXIECKFPVEKQLDLAALXVYWEMXDKNIIXFVHGEXDLKVQHSSYRQRARLLKDQLSLGNAALQITDVKLQDAGVYRCMXXXXXXXXITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKI

SEQ ID NO: 56 - protein, 390 aa, Homo sapiens
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 57 - protein, 112 aa, Homo sapiens
ALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 58 - protein, 413 aa, Homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSEAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 59 - protein, 112 aa, Homo sapiens
ALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 60 - protein, 412 aa, Homo sapiens
MKMHLQRALVVLALLNFATVSLSLSTCTTLDFGHIKKKRVEAIRGQILSKLRLTSPPEPTVMTHVPYQVLALYNSTRELLEEMHGEREEGCTQENTESEYYAKEIHKFDMIQGLAEHNELAVCPKGITSKVFRFNVSSVEKNRTNLFRAEFRVLRVPNPSSKRNEQRIELFQILRPDEHIAKQRYIGGKNLPTRGTAEWLSFDVTDTVREWLLRRESNLGLEISIHCPCHTFQPNGDILENIHEVMEIKFKGVDNEDDHGRGDLGRLKKQKDHHNPHLILMMIPPHRLDNPGQGGQRKKRALDTNYCFRNLEENCCVRPLYIDFRQDLGWKWVHEPKGYYANFCSGPCPYLRSADTTHSTVLGLYNTLNPEASASPCCVPQDLEPLTILYYVGRTPKVEQLSNMVVKSCKCS

SEQ ID NO: 61 - protein, 112 aa, Homo sapiens [sequence omitted]

SEQ ID NO: 62 - protein, 112 aa, synthetic construct
ALDTNYCFRNLEENCCVRPLYIDFRQDLGWKWVHEPKGYYANFCSGPCPYLRSADTTHSTVLGLYNTLNPEASASPSCVPQDLEPLTILYYVGRTPKVEQLSNMVVKSCKCS

SEQ ID NO: 63 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 64 - protein, 209 aa, Homo sapiens
RKGPPAALTLPRVQCRASRYPIAVDCSWTLPPAPNSTSPVSFIATYRLGMAARGHSWPCLQQTPTSTSCTITDVQLFSMAPYVLNVTAVHPWGSSSSFVPFITEHIIKPDPPEGVRLSPLAERQLQVQWEPPGSWPFPEIFSLKYWIRYKRQGAARFHRVGPIEATSFILRAVRPRARYYVQVAAQDLTDYGELSDWSLPATATMSLGK

SEQ ID NO: 65 - protein, 377 aa, Homo sapiens
MSMSPTVIILACLGFFLDQSVWAHVGGQDKPFCSAWPSAVVPQGGHVTLRCHYRRGFNIFTLYKKDGVPVPELYNRIFWNSFLISPVTPAHAGTYRCRGFHPHSPTEWSAPSNPLVIMVTGLYEKPSLTARPGPTVRAGENVTLSCSSQSSFDIYHLSREGEAHELRLPAVPSINGTFQADFPLGPATHGETYRCFGSFHGSPYEWSDPSDPLPVSVTGNPSSSWPSPTEPSFKTGIARHLHAVIRYSVAIILFTILPFFLLHRWCSKKKNAAVMNQEPAGHRTVNREDSDEQDPQEVTYAQLDHCIFTQRKITGPSQRSKRPSTDTSVCIELPNAEPRALSPAHEHHSQALMGSSRETTALSQTQLASSNVPAAGI

SEQ ID NO: 66 - protein, 273 aa, synthetic construct
MSMSPTVIILACLGFFLDQSVWAHVGGQDKPFCSAWPSAVVPQGGHVTLRCHYRRGFNIFTLYKKDGVPVPELYNRIFWNSFLISPVTPAHAGTYRCRGFHPHSPTEWSAPSNPLVIMVTGLYEKPSLTARPGPTVRAGENVTLSCSSQSSFDIYHLSREGEAHELRLPAVPSINGTFQADFPLGPATHGETYRCFGSFHGSPYEWSDPSDPLPVSVTGNPSSSWPSPTEPSFKTGIARHLHAVIRYSVAIILFTILPFFLLHRWCSKKKMLL

SEQ ID NO: 67 - protein, 342 aa, Homo sapiens
MSMSPTVIILACLGFFLDQSVWAHVGGQDKPFCSAWPSAVVPQGGHVTLRCHYRRGFNIFTLYKKDGVPVPELYNRIFWNSFLISPVTPAHAGTYRCRGFHPHSPTEWSAPSNPLVIMVTGLYEKPSLTARPGPTVRAGENVTLSCSSQSSFDIYHLSREGEAHELRLPAVPSINGTFQADFPLGPATHGETYRCFGSFHGSPYEWSDPSDPLPVSVTGNPSSSWPSPTEPSFKTDAAVMNQEPAGHRTVNREDSDEQDPQEVTYAQLDHCIFTQRKITGPSQRSKRPSTDTSVCIELPNAEPRALSPAHEHHSQALMGSSRETTALSQTQLASSNVPAAGI

SEQ ID NO: 68 - protein, 9 aa, synthetic construct
RLPKDFRIL

SEQ ID NO: 69 - protein, 9 aa, synthetic construct
KGPPAALTL

SEQ ID NO: 70 - protein, 9 aa, synthetic construct
KLPQAFYIL

SEQ ID NO: 71 - protein, 9 aa, synthetic construct
KLPAQFYIL

SEQ ID NO: 72 - protein, 10 aa, synthetic construct
RIPQGFGNLL

SEQ ID NO: 73 - protein, 9 aa, synthetic construct
KIPAQFYIL

SEQ ID NO: 74 - protein, 9 aa, synthetic construct
KGPAQFYIL

SEQ ID NO: 75 - protein, 9 aa, synthetic construct
RGPPQRPKL

SEQ ID NO: 76 - protein, 9 aa, synthetic construct
RIHDKAVAL

SEQ ID NO: 77 - protein, 9 aa, synthetic construct
RAIQKKIDL

SEQ ID NO: 78 - protein, 9 aa, synthetic construct
RLKKSADTL

SEQ ID NO: 79 - protein, 8 aa, synthetic construct
KSPPPMNL

SEQ ID NO: 80 - protein, 9 aa, synthetic construct
KYIHSANVL

SEQ ID NO: 81 - protein, 9 aa, synthetic construct
KIIDSGPQL

SEQ ID NO: 82 - protein, 10 aa, synthetic construct
TAVISIGNQL

SEQ ID NO: 83 - protein, 9 aa, synthetic construct
VVPKDRVAL

SEQ ID NO: 84 - protein, 9 aa, synthetic construct
RSPVYLTVL

SEQ ID NO: 85 - protein, 9 aa, synthetic construct
RHPKYKTEL

SEQ ID NO: 86 - protein, 9 aa, synthetic construct
HVPEHAVVL

SEQ ID NO: 87 - protein, 9 aa, synthetic construct
MQPTHPIRL

SEQ ID NO: 88 - protein, 9 aa, synthetic construct
KIAGYVTHL

SEQ ID NO: 89 - protein, 9 aa, synthetic construct
SYPTRIASL

SEQ ID NO: 90 - protein, 9 aa, synthetic construct
RLPDGRVVL

SEQ ID NO: 91 - protein, 9 aa, synthetic construct
MRPRKAFLL

SEQ ID NO: 92 - protein, 9 aa, synthetic construct
RLPKDFVDL

SEQ ID NO: 93 - protein, 9 aa, synthetic construct
VLPKLYVKL

SEQ ID NO: 94 - protein, 220 aa, synthetic construct
  VARIANT 8: note = where X is any amino acid other than Arg
FTVTVPKXLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 95 - protein, 220 aa, synthetic construct
  VARIANT 19: note = where X is any amino acid other than Thr
FTVTVPKDLYVVEYGSNMXIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 96 - protein, 220 aa, synthetic construct
  VARIANT 36: note = where X is any amino acid other than Ile
FTVTVPKDLYVVEYGSNMTIECKFPVEKQLDLAALXVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 97 - protein, 220 aa, synthetic construct
  VARIANT 42: note = where X is any amino acid other than Glu
FTVTVPKDLYVVEYGSNMTIECKFPVEKQLDLAALIVYWEMXDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 98 - protein, 220 aa, synthetic construct
  VARIANT 48: note = where X is any amino acid other than Gln
FTVTVPKDLYVVEYGSNMTIECKFPVEKQLDLAALIVYWEMEDKNIIXFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 99 - protein, 220 aa, synthetic construct
  VARIANT 54: note = where X is any amino acid other than Glu
FTVTVPKDLYVVEYGSNMTIECKFPVEKQLDLAALIVYWEMEDKNIIQFVHGEXDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 100 - protein, 220 aa, synthetic construct
  VARIANT 98: note = where X is any amino acid other than Ile
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMXSYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 101 - protein, 220 aa, synthetic construct
  VARIANT 101: note = where X is any amino acid other than Gly
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYXGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 102 - protein, 220 aa, synthetic construct
  VARIANT 102: note = where X is any amino acid other than Gly
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGXADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 103 - protein, 220 aa, synthetic construct
  VARIANT 103: note = where X is any amino acid other than Ala
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGXDYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 104 - protein, 220 aa, synthetic construct
  VARIANT 104: note = where X is any amino acid other than Asp
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGAXYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 105 - protein, 220 aa, synthetic construct
  VARIANT 105: note = where X is any amino acid other than Tyr
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADXKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 106 - protein, 220 aa, synthetic construct
  VARIANT 106: note = where X is any amino acid other than Lys
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYXRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 107 - protein, 220 aa, synthetic construct
  VARIANT 107: note = where X is any amino acid other than Arg
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKXITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNER

SEQ ID NO: 108 - protein, 179 aa, Homo sapiens
QLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 109 - protein, 160 aa, Homo sapiens
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 110 - protein, 243 aa, Homo sapiens
MGQTAGDLGWRLSLLLLPLLLVQAGVWGFPRPPGRPQLSLQELRREFTVSLHLARKLLAEVRGQAHRFAESHLPGVNLYLLPLGEQLPDVSLTFQAWRRLSDPERLCFISTTLQPFHALLGGLGTQGRWTNMERMQLWAMRLDLRDLQRHLRFQVLAAGFNLPEEEEEEEEEEEEERKGLLPGALGSALQGPAQVSWPQLLSTYRLLHSLELVLSRAVRELLLLSKAGHSVWPLGFPTLSPQP

SEQ ID NO: 111 - protein, 50 aa, synthetic construct
  VARIANT 1..50: note = Wherein up to 9 copies of GSGGS may be deleted
GSGGSGSGGSGSGGSGSGGSGSGGSGSGGSGSGGSGSGGSGSGGSGSGGS

SEQ ID NO: 112 - protein, 50 aa, synthetic construct
  VARIANT 1..50: note = Wherein up to 9 copies of GGGGS may be deleted
GGGGSGGGGSGGGGSGGGGSGGGGSGGGGSGGGGSGGGGSGGGGSGGGGS

SEQ ID NO: 113 - protein, 40 aa, synthetic construct
  VARIANT 1..40: note = Wherein up to 9 copies of GGGS may be deleted
GGGSGGGSGGGSGGGSGGGSGGGSGGGSGGGSGGGSGGGS

SEQ ID NO: 114 - protein, 4 aa, synthetic construct
GGSG

SEQ ID NO: 115 - protein, 5 aa, synthetic construct
GGSGG

SEQ ID NO: 116 - protein, 5 aa, synthetic construct
GSGSG

SEQ ID NO: 117 - protein, 5 aa, synthetic construct
GSGGG

SEQ ID NO: 118 - protein, 5 aa, synthetic construct
GGGSG

SEQ ID NO: 119 - protein, 5 aa, synthetic construct
GSSSG

SEQ ID NO: 120 - protein, 100 aa, synthetic construct
  VARIANT 1..100: note = Wherein up to 19 copies of EAAAK may be deleted
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 121 - protein, 102 aa, synthetic construct
  VARIANT 2..101: note = Wherein up to 19 copies of EAAAK may be deleted
AEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKA

SEQ ID NO: 122 - protein, 206 aa, synthetic construct
  VARIANT 2..101: note = Wherein up to 19 copies of EAAAK may be deleted
  VARIANT 106..205: note = Wherein up to 19 copies of EAAAK may be deleted
AEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKALEAEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKA

SEQ ID NO: 123 - protein, 5 aa, synthetic construct
EAAAK

SEQ ID NO: 124 - protein, 10 aa, synthetic construct
EAAAKEAAAK

SEQ ID NO: 125 - protein, 15 aa, synthetic construct
EAAAKEAAAKEAAAK

SEQ ID NO: 126 - protein, 46 aa, synthetic construct
AEAAAKEAAAKEAAAKEAAAKALEAEAAAKEAAAKEAAAKEAAAKA

SEQ ID NO: 127 - protein, 12 aa, synthetic construct
AEAAAKEAAAKA

SEQ ID NO: 128 - protein, 4 aa, synthetic construct
APAP

SEQ ID NO: 129 - protein, 8 aa, synthetic construct
APAPAPAP

SEQ ID NO: 130 - protein, 12 aa, synthetic construct
APAPAPAPAPAP

SEQ ID NO: 131 - protein, 16 aa, synthetic construct
APAPAPAPAPAPAPAP

SEQ ID NO: 132 - protein, 20 aa, synthetic construct
APAPAPAPAPAPAPAPAPAP

SEQ ID NO: 133 - protein, 4 aa, synthetic construct
KPKP

SEQ ID NO: 134 - protein, 8 aa, synthetic construct
KPKPKPKP

SEQ ID NO: 135 - protein, 12 aa, synthetic construct
KPKPKPKPKPKP

SEQ ID NO: 136 - protein, 16 aa, synthetic construct
KPKPKPKPKPKPKPKP

SEQ ID NO: 137 - protein, 20 aa, synthetic construct
KPKPKPKPKPKPKPKPKPKP

SEQ ID NO: 138 - protein, 4 aa, synthetic construct
EPEP

SEQ ID NO: 139 - protein, 8 aa, synthetic construct
EPEPEPEP

SEQ ID NO: 140 - protein, 12 aa, synthetic construct
EPEPEPEPEPEP

SEQ ID NO: 141 - protein, 16 aa, synthetic construct
EPEPEPEPEPEPEPEP

SEQ ID NO: 142 - protein, 20 aa, synthetic construct
EPEPEPEPEPEPEPEPEPEP

SEQ ID NO: 143 - protein, 5 aa, synthetic construct
GCGGS

SEQ ID NO: 144 - protein, 55 aa, synthetic construct
  VARIANT 6..55: note = Wherein up to 9 copies of GGGGS may be deleted
GCGGSGGGGSGGGGSGGGGSGGGGSGGGGSGGGGSGGGGSGGGGSGGGGSGGGGS

SEQ ID NO: 145 - protein, 5 aa, synthetic construct
CGGGS

SEQ ID NO: 146 - protein, 55 aa, synthetic construct
  VARIANT 6..55: note = Wherein up to 9 copies of GGGGS may be deleted
CGGGSGGGGSGGGGSGGGGSGGGGSGGGGSGGGGSGGGGSGGGGSGGGGSGGGGS

SEQ ID NO: 147 - protein, 5 aa, synthetic construct
GGCGS

SEQ ID NO: 148 - protein, 55 aa, synthetic construct
  VARIANT 6..55: note = Wherein up to 9 copies of GGGGS may be deleted
GGCGSGGGGSGGGGSGGGGSGGGGSGGGGSGGGGSGGGGSGGGGSGGGGSGGGGS

SEQ ID NO: 149 - protein, 5 aa, synthetic construct
GGGCS

SEQ ID NO: 150 - protein, 55 aa, synthetic construct
  VARIANT 6..55: note = Wherein up to 9 copies of GGGGS may be deleted
GGGCSGGGGSGGGGSGGGGSGGGGSGGGGSGGGGSGGGGSGGGGSGGGGSGGGGS

SEQ ID NO: 151 - protein, 5 aa, synthetic construct
GGGGC

SEQ ID NO: 152 - protein, 55 aa, synthetic construct
  VARIANT 6..55: note = Wherein up to 9 copies of GGGGS may be deleted
GGGGCGGGGSGGGGSGGGGSGGGGSGGGGSGGGGSGGGGSGGGGSGGGGSGGGGS

SEQ ID NO: 153 - protein, 5 aa, synthetic construct
  VARIANT 4: note = wherein X is any amino acid other than Pro
VPGXG